(19) United States
(12) Patent Application Publication
(10) Pub. No.: US 2009/0215758 A1
(43) Pub. Date: Aug. 27, 2009

(54) Use of 2,5-Disubstituted Thiazol-4-One Derivatives in Drugs

(75) Inventors: Robert Frank, Aachen (DE); Achim Kless, Aachen (DE); Ruth Jostock, Stolberg (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/915,156

(22) PCT Filed: May 17, 2006
(86) PCT No.: PCT/EP2006/004666

§ 371 Date: Jun. 10, 2008

(30) Foreign Application Priority Data
May 20, 2005 (DE) 10 2005 024 012.7

(51) Int. Cl. A61K 31/541; C07D 277/34; A61K 31/426; C07D 417/04; A61P 1/04; A61K 31/496

(52) U.S. Cl. 514/227.8; 548/186; 514/369; 544/58.7; 544/369; 514/254.02

(57) ABSTRACT

The invention relates to 2,5-disubstituted thiazol-4-one derivatives and to their use in the production of drugs, to methods for producing them and to drugs containing said compounds.

The present invention relates to 2,5-disubstituted thiazol-4-one derivatives and to the use thereof for producing medicaments, to processes for their preparation and to medicaments comprising these compounds.

The treatment of pain, especially of neuropathic pain, has great significance in medicine. There is a global demand for effective pain therapies. The urgent need for research into patient-oriented and targeted treatment of chronic and non-chronic states of pain, which is understood to mean the successful and satisfactory treatment of pain for the patient, is also documented in a large number of scientific studies which have recently appeared in the field of applied analgesics and fundamental research into nociception.

A suitable starting point for the treatment of pain, especially of neuropathic pain, is the vanilloid receptor of subtype 1 (VR1/TRPV1), which is frequently also referred to as the capsaicin receptor. This receptor is stimulated, inter alia, by vanilloids, for example capsaicin, heat and protons, and plays a central role in the development of pain. Furthermore, it is of significance for a multitude of further physiological and pathophysiological processes, for example migraines; depressions; neurodegenerative disorders; cognitive disorders; states of anxiety; epilepsy; coughing; diarrhea; pruritus; disorders of the cardiovascular system; disorders of food intake; medicament dependence; medicament abuse and especially urine incontinence.

It was therefore an object of the present invention to provide novel compounds which are suitable especially as active pharmaceutical ingredients in medicaments, preferably in medicaments for the treatment of disorders or diseases which are mediated at least partly by vanilloid receptors 1 (VR1/TRPV1 receptors).

It has now been found that, surprisingly, 2,5-disubstituted thiazol-4-one derivatives of the general formula I specified below are suitable for controlling pain and have an excellent affinity for the vanilloid receptor of subtype 1 (VR1/TRPV1 receptor), and are therefore suitable especially for prophylaxis and/or treatment of disorders or diseases which are mediated at least partly by vanilloid receptors 1 (VR1/TRPV1).

The present invention therefore provides for the use of at least one 2,5-disubstituted thiazol-4-one derivative of the general formula I

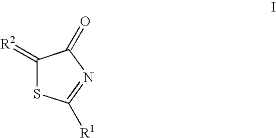

in which
R¹ is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   is an unsubstituted or at least monosubstituted aryl radical which may be fused to an unsubstituted or at least monosubstituted mono- or polycyclic ring system;
   is an NR³R⁴ group;
   is an NR⁵—C(═O)—R⁶ group;
   or is an NR⁷—C(═O)—NR⁸R⁹ group;
R² is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   is CH—U—X;
   is CH—CH₂—V—Y, CH—CH(CH₃)—V—Y, CH—CHCl—V—Y, CH—CHBr—V—Y, CH—CHF—V—Y or CH—CH(OH)—V—Y;
   or is CH—CH═C(CH₃)—W-Z, CH—CH═CH—W-Z, CH—C(CH₃)═CH—W-Z, CH—C(phenyl)=CH—W-Z, CH—CBr═CH—W-Z, CH—CCl═CH—W-Z, CH—CF═CH—W-Z, CH—C(OH)═CH—W-Z, CH—CH₂—CH₂—W-Z, CH—CH₂—CH(CH₃)—W-Z or CH—CH(CH₃)—CH₂—W-Z;
   where U, V and W may each be absent or are each independently a radical selected from the group consisting of O, S, N(H), N(CH₃), N(C₂H₅) and N[CH(CH₃)₂];
R³ is a linear or branched, saturated or unsaturated, unsubstituted or at least monosubstituted aliphatic radical optionally having heteroatom as a chain member;
   is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   or is an unsubstituted or at least monosubstituted aryl or heteroaryl radical which may be fused to an unsubstituted or at least monosubstituted mono- or polycyclic ring system and/or may be bonded via a linear or branched, unsubstituted or at least monosubstituted alkylene, alkenylene or alkynylene group;
R⁴ is a hydrogen radical
   or is a linear or branched, saturated or unsaturated, unsubstituted or at least monosubstituted aliphatic radical optionally having at least one heteroatom as a chain member;
R⁵, R⁷ and R⁹ are each independently
   a hydrogen radical
   or a linear or branched, saturated or unsaturated, unsubstituted or at least monosubstituted aliphatic radical;
R⁶ and R⁸ are each independently
   an unsubstituted or at least monosubstituted aryl or heteroaryl radical which may be fused to an unsubstituted or at least monosubstituted mono- or polycyclic ring system;
X is an aryl or heteroaryl radical which may be fused to an unsubstituted or at least monosubstituted mono- or polycyclic ring system and/or may be substituted by identical or different substituents R¹⁰;
Y is an aryl or heteroaryl radical which may be fused to an unsubstituted or at least monosubstituted mono- or polycyclic ring system and/or may be substituted by identical or different substituents R¹¹;
Z is a linear or branched, saturated or unsaturated, unsubstituted or at least monosubstituted aliphatic radical;
   or is an aryl or heteroaryl radical which may be fused to an unsubstituted or at least monosubstituted mono- or polycyclic ring system and/or may be substituted by identical or different substituents R¹²;
R¹⁰, R¹¹ and R¹² are each independently
   a radical selected from the group consisting of F, Cl, Br, I, —CN, —CF₃, —SF₅, —OH, —O—C₁₋₁₀-alkyl, —O—C₂₋₁₀-alkenyl, —NH₂, —NO₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)— benzo[b]furanyl and benzyl radicals may in each case be substituted;
   is a —C(═O)—NR¹³—(CH₂)ₘ—NR¹⁴—R¹⁵ group where m is 0, 1, 2, 3, 4 or 5;
   or is a —C(═O)—R¹⁶ group;
R¹³ and R¹⁴ are each independently
   a hydrogen radical
   or a linear or branched, saturated or unsaturated, unsubstituted or at least monosubstituted aliphatic radical;
R¹⁵ is an unsubstituted or at least monosubstituted aryl or heteroaryl radical which may be fused to an unsubstituted or at least monosubstituted mono- or polycyclic ring system; and
R¹⁶ is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates;
   for producing a medicament for prophylaxis and/or treatment of pain.

Aliphatic radicals in the context of this invention include acyclic saturated or unsaturated hydrocarbon radicals which may be branched or straight-chain and also unsubstituted or monosubstituted or polysubstituted identically or differently, having preferably from 1 to 20 (i.e. 1, 2, 3, 4, 5, 6, 7, 8, 9, 10, 11, 12, 13, 14, 15, 16, 17, 18, 19 or 20), more preferably from 1 to 12 (i.e. 1, 2, 3, 4, 5, 6, 7, 8, 9, 10, 11 or 12), most preferably from 1 to 6 (i.e. 1, 2, 3, 4, 5 or 6) carbon atoms, i.e. C₁₋₂₀-, C₁₋₁₂-, C₁₋₆-alkyls, C₂₋₂₀-, C₂₋₁₂-, C₂₋₆-alkenyls and C₂₋₂₀-, C₂₋₁₂-, C₂₋₆-alkynyls. Alkenyls have at least one C—C double bond and alkynyls have at least one C—C triple bond. Advantageously, aliphatic radicals may be selected from the group which comprises methyl, ethyl, n-propyl, isopropyl, n-butyl, isobutyl, sec-butyl, tert-butyl, n-pentyl, isopentyl, neopentyl, n-hexyl, 2-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosanyl, ethenyl(vinyl), ethynyl, propenyl (—CH₂CH═CH₂, —CH═CH—CH₃, —C(═CH₂)—CH₃), 2-methylpropenyl, propynyl (—CH₂—C≡H, —C≡C—CH₃), butenyl, butynyl, pentenyl, pentynyl, hexenyl, hexynyl, octenyl and octynyl.

The aforementioned aliphatic radicals may preferably have 1, 2 or 3 heteroatoms selected from the group comprising oxygen, sulfur and nitrogen, i.e. —N(H)— and —N(C₁₋₆-alkyl).

Examples of aliphatic radicals which have 1, 2 or 3 heteroatoms include —(CH₂)—(CH₂)—O—CH₃, —(CH₂)—(CH₂)—(CH₂)—O—CH₃, —(CH₂)—(CH₂)—(CH₂)—N(C₂H₅)—(C₂H₅), —(CH₂)—(CH₂)—S—CH₃, —(CH₂)—(CH₂)—(CH₂)—S—CH₃, —(CH₂)—(CH₂)—(CH₂)—N(CH₃)—(CH₃) and —(CH₂)—O—CH₃.

In connection with aliphatic radicals, the term “substituted”—unless defined differently elsewhere in the description or in the claims—in the context of this invention is understood to mean single or multiple substitution, preferably mono-, di-, tri-, tetra-, penta-, hexa-, hepta-, octa- or nonasubstitution, of one or more hydrogen atoms by, for example, F, Cl, Br, I, —CN, —NO₂, —OH, —SH and —NH₂, where the multiple substitution is multiple, for example double or triple, either on different or on the same atoms, for example triple on the same carbon atom as in the case of —CF₃ or —CH₂CF₃, or on different positions as in the case of —CH(OH)—CH═CCl—CH₂Cl. Multiple substitution can be effected with the same or different substituents. Particularly preferred substituted aliphatic radicals are —CF₃, —C₂F₅, —CH₂F, —CHF₂, —CF₂—CF₂—CF₃, —CH₂—Cl, —CH₂—Br, —CH₂—CH₂—Cl, —CH₂—CH₂—Br, —CH₂—CH₂—CH₂—Br and —CH₂—CH₂—CH₂—Cl.

Cycloaliphatic radicals in the context of this invention are cyclic saturated or unsaturated hydrocarbon radicals having preferably 3, 4, 5, 6, 7, 8, 9, 10, 11, 12, 13, 14, 15 or 16, more preferably 3, 4, 5, 6, 7 or 8 carbon atoms, where each radical may be unsubstituted or monosubstituted or polysubstituted identically or differently. Cycloaliphatic radicals may preferably have 1, 2, 3, 4 or 5 heteroatoms selected independently from the group consisting of oxygen, nitrogen (NH) and sulfur.

Examples of cycloaliphatic radicals which may be fused to a monocyclic or polycyclic ring system include cyclopropyl, cyclobutyl, cyclopentyl, cyclohexyl, cyclooctyl, cyclononyl, cyclodecyl, cycloundecyl, cyclododecyl, cycloheptyl, cyclopentenyl, cyclohexenyl, cycloheptenyl, [6,6]-dimethyl-[3.1.1]-bicycloheptyl, adamantyl, imidazolidinyl, tetrahydrofuranyl, tetrahydrothiophenyl, pyrrolidinyl, piperidinyl, morpholinyl, piperazinyl, thiomorpholinyl, tetrahydropyranyl, azepanyl, diazepanyl, dithiolanyl, indanyl, indenyl, (1,4)-benzodioxanyl, (1,2,3,4)-tetrahydronaphthyl, (1,2,3,4)-tetrahydroquinolinyl, (1,2,3,4)-tetrahydroisoquinolinyl, (1,2,3,4)-tetrahydroquinazolinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl, [3,4]-dihydro-2H-1,4-benzoxazinyl, (2,3)-dihydro-1H-isoindolyl, (2,3)-dihydroindolyl and (1,3)-thiazolidinyl.

A mono- or polycyclic ring system is understood in the context of the present invention to mean mono-polycyclic hydrocarbon radicals which may be saturated or unsaturated and optionally have 1, 2, 3, 4 or 5 heteroatom(s) as ring member(s), which are each independently selected from the group consisting of oxygen, nitrogen and sulfur. Such a mono- or polycyclic ring system may, for example, be fused to an aryl radical or a heteroaryl radical.

When a polycyclic ring system, for example a bicyclic ring system, is present, the different rings may each independently have a different degree of saturation, i.e. be saturated or unsaturated. A polycyclic ring system is preferably a bicyclic ring system.

Examples of aryl radicals which are fused to a mono- or polycyclic ring system include [1,3]-benzodioxolyl, [1,4]-benzodioxanyl, [1,2,3,4]-tetrahydronaphthyl, [1,2,3,4]-tetrahydroquinolinyl, [1,2,3,4]-tetrahydroquinazolinyl and [3,4]-dihydro-2H-1,4-benzoxazinyl.

In connection with cycloaliphatic radicals and mono- or polycyclic ring systems, the term “substituted”—unless defined differently elsewhere in the description or in the claims—in the context of this invention is understood to mean the single or multiple substitution, preferably the mono-, di-, tri-, tetra-, penta-, hexa-, hepta-, octa- or nonasubstitution, of one or more hydrogen atoms by, for example, oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH-phenyl, —NH-pyridinyl, —N(C₁₋₅-alkyl)-phenyl, —N(C₁₋₅-alkyl)-pyridinyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, [1,2,5]-thiadiazolyl, pyridazinyl, —(CH₂)-benzo[b]furanyl, —O-phenyl, —O-benzyl, phenyl and benzyl, where the cyclic moiety of the —NH-phenyl, —NH-pyridinyl, —N(C₁₋₅-alkyl)-phenyl, —N(C₁₋₅-alkyl)-pyridinyl, pyridinyl, cyclopentyl, [1,2,5]-thiadiazolyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

For the purposes of the present invention, the term “aryl radical” should be understood to mean a radical which is preferably selected from the group which comprises phenyl, naphthyl, phenanthrenyl and anthracenyl, and is unsubstituted or mono- or polysubstituted identically or differently. Aryl is more preferably an unsubstituted or monosubstituted or identically or differently polysubstituted, for example bi-, tri-, tetra- or pentasubstituted, phenyl, 1-naphthyl or 2-naphthyl.

In the context of the present invention, heteroaryl radicals are those heterocycles which are heteroaromatic. Heteroaryl radicals are preferably 5- to 14-membered, i.e. 5-, 6-, 7-, 8-, 9-, 10-, 11-, 12-, 13- or 14-membered, and have preferably 1, 2, 3, 4 or 5 heteroatoms selected independently from the group comprising oxygen, nitrogen and sulfur. Each heteroaryl radical may be present unsubstituted or monosubstituted or polysubstituted for example bi-, tri-, tetra- or pentasubstituted, identically or differently.

Examples of heteroaryl radicals in the context of the invention include thiophenyl, furanyl, pyrrolyl, pyrazolyl, pyranyl, pyridinyl, imidazolyl, indolyl, isoindolyl, benzo[b]furanyl, benzo[b]thiophenyl, thiazolyl, oxazolyl, isoxazolyl, pyridazinyl, pyrazinyl, pyrimidinyl, indazolyl, quinazolinyl, quinolinyl, isoquinolinyl, benzimidazolinyl, benzoxazolyl, benzisoxazolyl, benzothiazolyl, benzo[2,1,3]thiadiazolyl, [1,2,3]-benzothiadiazolyl, [2,1,3]-benzoxadiazolyl and [1,2,3]-benzoxadiazolyl.

In relation to aryl and heteroaryl radicals, “substituted” in the context of this invention is understood to mean the single or multiple substitution, for example mono-, di-, tri-, tetra- or pentasubstitution, of one or more hydrogen atoms of the ring system by suitable substituents. When the definition of these suitable substituents in connection with aryl or heteroaryl radicals is not defined elsewhere in the description or in the claims, suitable substituents are F, Cl, Br, I, —CN, —CF₃, —SF₅, —OH, —O—C₁₋₁₀-alkyl, —O—C₁₋₁₀-alkenyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —O—S—(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)— benzo[b]furanyl, —O-phenyl, —O-benzyl, phenyl and benzyl, where the cyclic moiety of the pyridinyl, cyclopentyl, cyclohexyl, pyridazinyl, —S(═O)₂-phenyl, —O—S—(═O)₂-phenyl, —O-phenyl, —O-benzyl, phenyl, —(CH₂)-benzo[b]furanyl and benzyl radicals may in each case preferably be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl. The multiple substitution is effected with the same or with different substituents.

The aforementioned linear or branched alkylene, alkenylene or alkynylene groups preferably have from 1 to 5 carbon atoms, i.e. they are C₁₋₅-alkylene, C₂₋₅-alkenylene or C₂₋₅-alkynylene groups, each of which may be unsubstituted or substituted by preferably 1, 2, 3, 4 or 5 substituents selected independently from the group consisting of F, Cl, Br, —OH, —SH, —NH₉, —CN, —NO₉ and phenyl, where the phenyl radical may preferably be substituted by 1, 2, 3, 4 or 5 substituents selected independently from the group consisting of methyl, ethyl, n-propyl, isopropyl, n-butyl, isobutyl, sec-butyl, tert-butyl, n-pentyl, isopentyl and neopentyl.

Alkylene groups may more preferably be selected from the group consisting of —(CH₂)—, —(CH₂)₂—, —C(H)(CH₃)—, —C(CH₃)₂—, —(CH₂)₃—, —(CH₂)₄—, —(CH₂)₅—, —C(H)(CH₃)—(CH₂)—, —C(H)(C₂H₅)—(CH₂)—, —C(phenyl)₂- and —C(H)(phenyl).

Alkenylene groups may more preferably be selected from the group consisting of —CH═CH—, —C(CH₃)═CH—, —C(C₂H₅)═CH—, —CH═C(CH₃)—, —CH═C(C₂H₅)—, —CH═C(phenyl)-, —CH═C(p-tolyl), —C(phenyl)=CH— and —C(p-tolyl)=CH—.

An alkynylene group is more preferably a —C≡C— group.

Particular preference is given to the use of at least one 2,5-disubstituted thiazol-4-one derivative of the above-specified general formula I in which
R¹ is one of the following radicals

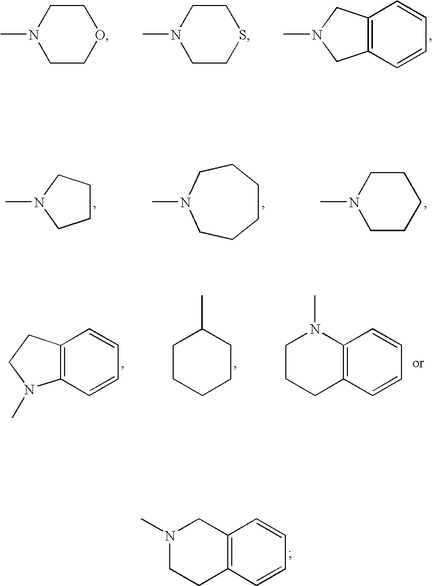

where the radical may be substituted by 1, 2 or 3 substituents selected independently from the group consisting of —OH, —O—CH₃, —O—C₂H₅, methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl and phenyl; where the cyclic moiety of the phenyl radical may in each case be substituted by 1, 2 or 3 substituents selected independently from the group consisting of F, Cl, Br and —CF₃;
is one of the following radicals

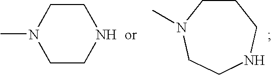

where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is a radical selected from the group consisting of phenyl and naphthyl, where the radical may in each case optionally be substituted by 1, 2, 3, 4 or 5 substituents selected independently from the group consisting of —OH, —O—CH₃ and —O—C₂H₅;
is an NR³R⁴ group
or is an NR⁷—C(═O)—NR⁸R⁹ group;
R² is a 1,3-dihydroindol-2-onyl or a 1,3-dihydroindol-2-thionyl radical;
   is CH—X;

is CH—CH₂—Y, CH—CH(CH₃)—Y, CH—CHCl—Y, CH—CHBr—Y, CH—CHF—Y or CH—CH(OH)—Y;

or is CH—CH═C(CH₃)-Z, CH—CH═CH-Z, CH—CH═CH—S-Z, CH—CH═CH—O-Z, CH—CH═CH—N(CH₃)-Z, CH—C(CH₃)═CH-Z, CH—C(phenyl)=CH-Z, CH—CBr═CH-Z, CH—CCl═CH-Z, CH—CF═CH-Z, CH—C(OH)═CH-Z, CH—CH₂—CH₂-Z, CH—CH₂—CH(CH₃)-Z or CH—CH(CH₃)—CH₂-Z;
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
   is a radical selected from the group consisting of cyclopentyl, cyclohexyl, cycloheptyl, cyclopentenyl, cyclohexenyl and cycloheptenyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)— phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl;
   or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R⁴ is a hydrogen radical
   or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁷ and R⁸ are each independently
   a hydrogen radical
   or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁹ is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CH₃, —O—C₂H₅, —O—CF₃, —S—CF₃, methyl, ethyl, n-propyl, isopropyl, n-butyl, sec-butyl, isobutyl and tert-butyl;
X is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹⁰;
Y is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹¹;
Z is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl;
   or is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹²;
R¹⁰ is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—CH₃, —NH—C(═O)—C₂H₅, —NH—C(═O)—C(CH₃)₃, —O—S(═O)₂-phenyl, —O-phenyl, —O-benzyl, phenyl and benzyl;
   is a —C(═O)—NR¹³—(CH₂)ₘ—NR¹⁴—R¹⁵ group where m is 0, 1, 2 or 3;
   or is a —C(═O)—R¹⁶ group;
R¹¹ is a radical selected from the group consisting of F, Cl, Br, —SF₅, methyl, ethyl, n-propyl, isopropyl, n-butyl, sec-butyl, tert-butyl, n-pentyl, n-hexyl, n-heptyl and n-octyl;
R¹² is a radical selected from the group consisting of F, Cl, Br, —SF₅, —CF₃, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, methyl, ethyl, n-propyl, isopropyl, n-butyl, sec-butyl, tert-butyl, n-pentyl, n-hexyl, n-heptyl and n-octyl;
R¹³ and R¹⁴ are each a hydrogen radical;
R¹⁵ is a radical selected from the group consisting of phenyl and pyridinyl, where the radical may in each case optionally be substituted by 1, 2, 3, 4 or 5 substituents selected independently from the group consisting of F, Cl, Br, —CF₃, —O—CF₃, —S—CF₃, methyl, ethyl, n-propyl, isopropyl, n-butyl, sec-butyl, isobutyl and tert-butyl; and
R¹⁶ is a radical selected from the group consisting of thiomorpholinyl, azepanyl, morpholinyl, (2,3)-dihydro-1H-isoindolyl, (2,3)-dihydroindolyl, piperidinyl and pyrrolidinyl; where the radical may be substituted by 1, 2 or 3 substituents selected independently from the group consisting of —OH, methyl, ethyl, n-propyl, isopropyl, n-butyl, sec-butyl, isobutyl, tert-butyl, benzyl and phenyl; where the cyclic moiety of the benzyl and phenyl radicals may in each case be substituted by 1, 2, 3, 4 or 5 substituents selected independently from the group consisting of F, Cl, Br and —CF₃;
   or is one of the following radicals

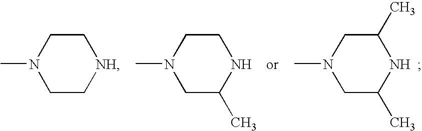

where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tertbutyl, —O—CH₃, —O—C₂H₅, —O—CF₃ and —S—CF₃;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates.

Very particular preference is given to the use of at least one 2,5-disubstituted thiazol-4-one derivative of the above-specified general formula I in which
R¹ is one of the following radicals

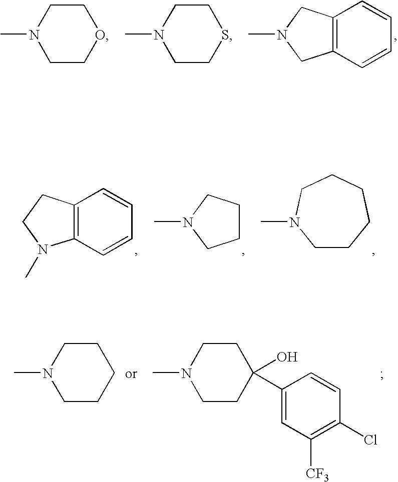

is the following radical

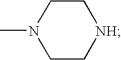

where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tertbutyl, —O—CH₃, —O—C₂H₅, —O—CF₃ and —S—CF₃;
is a radical selected from the group consisting of phenyl and naphthyl, where the radical may in each case optionally be substituted by 1, 2, 3, 4 or 5 substituents selected independently from the group consisting of —OH, —O—CH₃ and —O—C₂H₅;
is an NR³R⁴ group
or is an NR⁷—C(═O)—NR⁸R⁹ group;
R² is a 1,3-dihydroindol-2-onyl or a 1,3-dihydroindol-2-thionyl radical;
   is CH—X;
   is CH—CH₂—Y, CH—CH(CH₃)—Y or
   CH—CH(OH)—Y;
   or is CH—CH═C(CH₃)-Z, CH—CH═CH-Z, CH—CH═CH—S-Z, CH—C(CH₃)═CH-Z, CH—C(phenyl)=CH-Z,
   CH—CBr═CH-Z, CH—CCl═CH-Z, CH—CF═CH-Z, CH—CH₂—CH₂-Z, CH—CH₂—CH(CH₃)-Z or CH—CH(CH₃)—CH₂-Z;
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
   or is a radical selected from the group consisting of phenyl, naphthyl, benzyl, (1,3)-benzodioxolyl and (1,4)-benzodioxanyl; where the radical may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R⁴ is a hydrogen radical
   or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁷ and R⁸ are each a hydrogen radical;
R⁹ is a phenyl radical which may be substituted by 1, 2, 3, 4 or 5 substituents selected independently from the group consisting of F, Cl, Br and —CF₃;
X is a radical selected from the group consisting of phenyl, naphthyl, phenanthrenyl, (1,3)-benzodioxolyl, (1,4)-benzodioxanyl, thiophenyl, furanyl, pyridinyl, pyridazinyl, pyrazinyl, pyrimidinyl, quinolinyl and isoquinolinyl; which may be substituted by 1, 2, 3, 4 or 5 identical or different substituents R¹⁰;
Y is a phenyl or naphthyl radical which may be substituted by 1, 2, 3 4 or 5 identical or different substituents R¹¹;
Z is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl and n-octyl;
   or is a phenyl or naphthyl radical which may be substituted by 1, 2, 3, 4 or 5 identical or different substituents R¹²;
R¹⁰ is a radical selected from the group consisting of F, Cl, Br, I, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —O—CF₃, —S—CF₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —N(CH₃)₂, —N(C₂H₅)₂, —NH—CH₃, —NH—C₂H₅, —NH—C(═O)—CH₃, —O—S(═O)₂-phenyl, —O-phenyl, —O-benzyl and phenyl;
   is a —C(═O)—NR¹³—(CH₂)ₘ—NR¹⁴—R¹⁵ group where m is 0, 1, 2 or 3;
   or is a —C(═O)—R¹⁶ group;
R¹¹ is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl;
R¹² is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl;
R¹³ and R¹⁴ are each a hydrogen radical;
R¹⁵ is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CF₃, —S—CF₃, methyl, ethyl, n-propyl, isopropyl, n-butyl, sec-butyl, isobutyl and tert-butyl; and
R¹⁶ is one of the following radicals

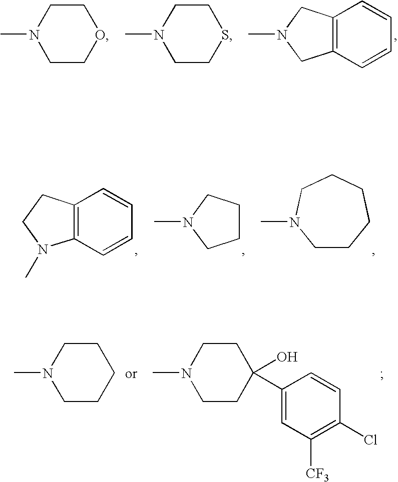

or is one of the following radicals

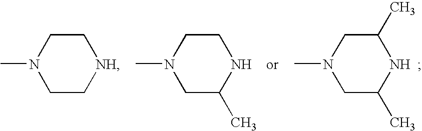

where the hydrogen atom of the —NH group may be replaced by a substituent selected from the group consisting of pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tertbutyl, —O—CH₃, —O—C₂H₅, —O—CF₃ and —S—CF₃;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates.

Even more preferred is the use of at least one 2,5-disubstituted thiazol-4-one derivative of the above-specified general formula I selected from the group consisting of:
1 5-[3-(4-methoxyphenyl)but-2-enylidene]-2-morpholin-4-ylthiazol-4-one
2 2-morpholin-4-yl-5-phenethylidenethiazol-4-one
3 2-morpholin-4-yl-5-(2-o-tolylethylidene)thiazol-4-one
4 2-morpholin-4-yl-5-(3-phenylallylidene)thiazol-4-one
5 2-morpholin-4-yl-5-(3-phenylbutylidene)thiazol-4-one
6 2-morpholin-4-yl-5-(3-phenylpropylidene)thiazol-4-one
7 5-[3-(4-fluorophenyl)allylidene]-2-morpholin-4-ylthiazol-4-one
8 N-[4-(2-morpholin-4-yl-4-oxo-4H-thiazol-5-ylidenemethyl)phenyl]acetamide
9 5-benzo[1,3]dioxol-5-ylmethylene-2-morpholin-4-ylthiazol-4-one
10 2-morpholin-4-yl-5-(3-m-tolylallylidene)thiazol-4-one
11 5-(3-iodo-4,5-dimethoxybenzylidene)-2-morpholin-4-ylthiazol-4-one
12 2-morpholin-4-yl-5-(3-phenylsulfanylallylidene)thiazol-4-one
13 5-(3-benzyloxy-4-methoxybenzylidene)-2-morpholin-4-ylthiazol-4-one
14 5-(4-methylbenzylidene)-2-thiomorpholin-4-ylthiazol-4-one
15 N-[4-(2-morpholin-4-yl-4-oxo-4H-thiazol-5-ylidenemethyl)phenyl]amine
16 5-(4-butoxybenzylidene)-2-morpholin-4-ylthiazol-1-one
17 2-morpholin-4-yl-5-(4-phenoxybenzylidene)thiazol-4-one
18 2-morpholin-4-yl-5-(3-phenoxybenzylidene)thiazol-4-one
19 2-morpholin-4-yl-5-(3-p-tolylallylidene)thiazol-4-one
20 5-(3-benzyloxybenzylidene)-2-morpholin-4-ylthiazol-4-one
21 5-(4-benzyloxybenzylidene)-2-morpholin-4-ylthiazol-4-one
22 2-thiomorpholin-4-yl-5-(2-trifluoromethylbenzylidene)thiazol-4-one
23 5-(4-bromobenzylidene)-2-thiomorpholin-4-ylthiazol-4-one
24 2-thiomorpholin-4-yl-5-(4-trifluoromethoxybenzylidene)thiazol-4-one
25 5-(3-phenylallylidene)-2-thiomorpholin-4-ylthiazol-4-one
26 2-morpholin-4-yl-5-octylidenethiazol-4-one
27 5-(4-benzyloxy-3-methoxybenzylidene)-2-morpholin-4-ylthiazol-4-one
28 5-(3,4-bis(benzyloxy)benzylidene)-2-morpholin-4-ylthiazol-4-one
29 5-butylidene-2-morpholin-4-ylthiazol-4-one
30 2-[4-(3-chloropyridin-2-yl)piperazin-1-yl]-5-(4-methylbenzylidene)thiazol-4-one
31 2-[4-(3-chloropyridin-2-yl)piperazin-1-yl]-5-(3-phenylallylidene)thiazol-4-one
32 5-hexylidene-2-morpholin-4-ylthiazol-4-one
33 2-morpholin-4-yl-5-(3-p-tolyl but-2-enylidene)thiazol-4-one
34 5-[3-(4-tert-butylphenyl)but-2-enylidene]-2-morpholin-4-ylthiazol-4-one
35 5-(4-methoxynaphthalen-1-ylmethylene)-2-morpholin-4-ylthiazol-4-one
36 2-morpholin-4-yl-5-(4-trifluoromethoxybenzylidene)thiazol-4-one
37 2-[4-(3-methoxyphenyl)piperazin-1-yl]-5-(3-phenylallylidene)thiazol-4-one
38 5-[3-(4-tert-butylphenyl)allylidene]-2-[4-(3-methoxyphenyl)piperazin-1-yl]-thiazol-4-one
39 5-(4-hydroxy-3-methoxybenzylidene)-2-thiomorpholin-4-ylthiazol-4-one
40 5-[3-(4-hydroxy-3-methoxyphenyl)allylidene]-2-thiomorpholin-4-ylthiazol-4-one
41 5-naphthalen-1-ylmethylene-2-thiomorpholin-4-ylthiazol-4-one
42 5-benzylidene-2-thiomorpholin-4-ylthiazol-4-one
43 2-morpholin-4-yl-5-[3-(4-trifluoromethylphenyl)but-2-enylidene]thiazol-4-one
44 2-morpholin-4-yl-5-[3-(3-trifluoromethylphenyl)but-2-enylidene]thiazol-4-one
45 5-(3-methylbutylidene)-2-morpholin-4-ylthiazol-4-one
46 2-[4-(3-chloropyridin-2-yl)piperazin-1-yl]-5-(2-trifluoromethylbenzylidene)thiazol-4-one
47 5-(4-bromobenzylidene)-2-[4-(3-chloropyridin-2-yl)piperazin-1-yl]-thiazol-4-one
48 5-benzylidene-2-[4-(3-chloropyridin-2-yl)piperazin-1-yl]thiazol-4-one
49 2-[4-(3-chloropyridin-2-yl)piperazin-1-yl]-5-naphthalen-2-ylmethylenethiazol-4-one
50 5-(2,3-diphenylallylidene)-2-morpholin-4-ylthiazol-4-one
51 2-morpholin-4-yl-5-phenanthren-9-ylmethylenethiazol-4-one
52 2-morpholin-4-yl-5-quinolin-3-ylmethylenethiazol-4-one
53 2-(4-ethylpiperazin-1-yl)-5-(4-methylbenzylidene)thiazol-4-one
54 5-naphthalen-2-ylmethylene-2-thiomorpholin-4-ylthiazol-4-one
55 2-[4-(4-methoxyphenyl)piperazin-1-yl]-5-(3-phenylallylidene)thiazol-4-one
56 2-[4-(2-chlorophenyl)piperazin-1-yl]-5-(3-phenylallylidene)thiazol-4-one
57 5-(3-phenylallylidene)-2-(4-phenylpiperazin-1-yl)thiazol-4-one
58 5-decylidene-2-morpholin-4-ylthiazol-4-one
59 5-[3-(4-tert-butylphenyl)allylidene]-2-morpholin-4-ylthiazol-4-one
60 2-(2-morpholin-4-yl-4-oxo-4H-thiazol-5-ylidenemethyl)benzoic acid
61 5-(2-bromo-3-phenylallylidene)-2-[4-(3-chloropyridin-2-yl)piperazin-1-yl]thiazol-4-one
62 5-[3-(4-tert-butylphenyl)allylidene]-2-[4-(3-chloropyridin-2-yl)piperazin-1-yl]-thiazol-4-one
63 2-(4-benzylpiperazin-1-yl)-5-(4-methylbenzylidene)thiazol-4-one
64 2-(4-phenylpiperazin-1-yl)-5-quinolin-3-ylmethylenethiazol-4-one
65 2-[4-(3-chloropyridin-2-yl)piperazin-1-yl]-5-(2-methyl-3-phenylallylidene)thiazol-4-one
66 2-[4-(3-chloropyridin-2-yl)piperazin-1-yl]-5-naphthalen-1-ylmethylenethiazol-4-one
67 5-(2-chloro-3-phenylallylidene)-2-[4-(3-chloropyridin-2-yl)piperazin-1-yl]thiazol-4-one
68 5-(4-hydroxy-3-methoxybenzylidene)-2-(4-phenylpiperazin-1-yl)thiazol-4-one
69 5-phenanthren-9-ylmethylene-2-(4-phenylpiperazin-1-yl)thiazol-4-one
70 5-(6-methoxynaphthalen-2-ylmethylene)-2-(4-phenylpiperazin-1-yl)thiazol-4-one
71 5-naphthalen-1-ylmethylene-2-(4-phenylpiperazin-1-yl)thiazol-4-one
72 5-(4-bromobenzylidene)-2-(4-phenylpiperazin-1-yl)thiazol-4-one
73 5-(4-methylbenzylidene)-2-(4-phenylpiperazin-1-yl)thiazol-4-one
74 2-(4-phenyl piperazin-1-yl)-5-(2-trifluoromethylbenzylidene)thiazol-4-one
75 3-(4-oxo-2-thiomorpholin-4-yl-4H-thiazol-5-ylidene)-1,3-dihydroindol-2-one
76 3-(2-morpholin-4-yl-4-oxo-4H-thiazol-5-ylidene)-1,3-dihydroindol-2-one
77 2-[4-(2-fluorophenyl)piperazin-1-yl]-5-(4-methylbenzylidene)thiazol-4-one
78 5-(4-tert-butylbenzylidene)-2-[4-(2-fluorophenyl)piperazin-1-yl]thiazol-4-one
79 2-[4-(2-fluorophenyl)piperazin-1-yl]-5-(3-phenylallylidene)thiazol-4-one
80 5-[3-(4-tert-butylphenyl)allylidene]-2-[4-(2-fluorophenyl)piperazin-1-yl]thiazol-4-one
81 2-[4-(2-fluorophenyl)piperazin-1-yl]-5-(4-trifluoromethylbenzylidene)thiazol-4-one
82 2-[4-(2-fluorophenyl)piperazin-1-yl]-5-(4-trifluoromethoxybenzylidene)thiazol-4-one
83 5-(2-chloro-3-phenylallylidene)-2-[4-(2-fluorophenyl)piperazin-1-yl]thiazol-4-one
84 2-[4-(2-fluorophenyl)piperazin-1-yl]-5-naphthalen-1-ylmethylenethiazol-4-one
85 5-(2-chloro-3-phenylallylidene)-2-thiomorpholin-4-ylthiazol-4-one
86 3-(4-oxo-2-thiomorpholin-4-yl-4H-thiazol-5-ylidenemethyl)benzoic acid
87 4-(4-oxo-2-thiomorpholin-4-yl-4H-thiazol-5-ylidenemethyl)benzoic acid
88 5-{3-[4-(3-chloropyridin-2-yl)piperazine-1-carbonyl]benzylidene}-2-thiomorpholin-4-ylthiazol-4-one
89 5-{4-[4-(3-chloropyridin-2-yl)piperazine-1-carbonyl]benzylidene}-2-thiomorpholin-4-ylthiazol-4-one
90 2-[4-(3-chloropyridin-2-yl)piperazin-1-yl]-5-quinolin-3-ylmethylenethiazol-4-one
91 2-[4-(3-chloropyridin-2-yl)piperazin-1-yl]-5-(4-trifluoromethylbenzylidene)thiazol-4-one
92 2-(2,3-dihydroindol-1-yl)-5-(4-methylbenzylidene)thiazol-4-one
93 2-(2,3-dihydroindol-1-yl)-5-(4-trifluoromethylbenzylidene)thiazol-4-one
94 5-[4-(morpholine-4-carbonyl)benzylidene]-2-thiomorpholin-4-ylthiazol-4-one
95 2-[4-(3-chloropyridin-2-yl)piperazin-1-yl]-5-(4-hydroxy-3-methoxybenzylidene)thiazol-4-one
96 1-[4-oxo-5-(4-trifluoromethylbenzylidene)-4,5-dihydrothiazol-2-yl]-3-(2-trifluoromethylphenyl)urea
97 2-(4-benzylpiperazin-1-yl)-5-quinolin-3-ylmethylenethiazol-4-one
98 2-(4-benzylpiperazin-1-yl)-5-naphthalen-2-ylmethylenethiazol-4-one
99 2-(4-benzylpiperazin-1-yl)-5-(3-phenoxybenzylidene)thiazol-4-one
100 2-(4-benzylpiperazin-1-yl)-5-(4-trifluoromethylbenzylidene)thiazol-4-one
101 1-[5-(4-methylbenzylidene)-4-oxo-4,5-dihydrothiazol-2-yl]-3-(2-trifluoromethylphenyl)urea
102 2-(4-benzylpiperazin-1-yl)-5-naphthalen-1-ylmethylenethiazol-4-one
103 2-(4-benzylpiperazin-1-yl)-5-(6-methoxynaphthalen-2-ylmethylene)thiazol-4-one
104 2-(4-benzylpiperazin-1-yl)-5-(4-tert-butylbenzylidene)thiazol-4-one
105 1-[4-oxo-5-(3-phenylallylidene)-4,5-dihydrothiazol-2-yl]-3-(2-trifluoromethylphenyl)urea
106 1-[4-oxo-5-(4-trifluoromethylbenzylidene)-4,5-dihydrothiazol-2-yl]-3-(4-trifluoromethylphenyl)urea
107 1-[5-(4-methylbenzylidene)-4-oxo-4,5-dihydrothiazol-2-yl]-3-(4-trifluoromethylphenyl)urea
108 5-{3-[4-(6-chloropyridin-2-yl)piperazine-1-carbonyl]benzylidene}-2-thiomorpholin-4-ylthiazol-4-one
109 2-(4-benzylpiperazin-1-yl)-5-(4-trifluoromethoxybenzylidene)thiazol-4-one
110 5-{3-[4-(3-chloropyridin-2-yl)-3-methylpiperazine-1-carbonyl]benzylidene}-2-thiomorpholin-4-ylthiazol-4-one
111 N-[3-(3-chloro-5-trifluoromethylpyridin-2-ylamino)propyl]-3-(4-oxo-2-thiomorpholin-4-yl-4H-thiazol-5-ylidenemethyl)benzamide
112 3-(4-oxo-2-thiomorpholin-4-yl-4H-thiazol-5-ylidenemethyl)benzoic acid N′-(2-chloro-4-trifluoromethylphenyl)hydrazide
113 5-{3-[4-(3-chloro-5-trifluoromethylpyridin-2-yl)piperazine-1-carbonyl]-benzylidene}-2-thiomorpholin-4-ylthiazol-4-one
114 3-(4-oxo-2-thiomorpholin-4-yl-4H-thiazol-5-ylidenemethyl)benzoic acid N′-(3-chloro-5-trifluoromethylpyridin-2-yl)hydrazide
115 5-{3-[4-(4-chloro-[1,2,5]thiadiazol-3-yl)piperazine-1-carbonyl]benzylidene}-2-thiomorpholin-4-ylthiazol-4-one
116 3-(4-oxo-2-thiomorpholin-4-yl-4H-thiazol-5-ylidenemethyl)benzoic acid N′-(2-chloro-5-trifluoromethylphenyl)hydrazide
117 2-[4-(4-chloro-[1,2,5]thiadiazol-3-yl)piperazin-1-yl]-5-(4-methylbenzylidene)thiazol-4-one
118 2-[4-(4-chloro-[1,2,5]thiadiazol-3-yl)piperazin-1-yl]-5-(4-trifluoromethylbenzylidene)thiazol-4-one
119 2-[4-(4-chloro-[1,2,5]thiadiazol-3-yl)piperazin-1-yl]-5-(4-trifluoromethoxybenzylidene)thiazol-4-one
120 5-(4-tert-butylbenzylidene)-2-[4-(4-chloro-[1,2,5]thiadiazol-3-yl)piperazin-1-yl]thiazol-4-one
121 2-[4-(4-chloro-[1,2,5]thiadiazol-3-yl)piperazin-1-yl]-5-(3-phenylallylidene)thiazol-4-one
122 5-[3-(4-tert-butylphenyl)allylidene]-2-[4-(4-chloro-[1,2,5]thiadiazol-3-yl)-piperazin-1-yl]thiazol-4-one
123 2-[4-(4-chloro-[1,2,5]thiadiazol-3-yl)piperazin-1-yl]-5-naphthalen-1-ylmethylenethiazol-4-one
124 2-[4-(4-chloro-[1,2,5]thiadiazol-3-yl)piperazin-1-yl]-5-(3-phenoxybenzylidene)thiazol-4-one
125 2-[4-(4-chloro-[1,2,5]thiadiazol-3-yl)piperazin-1-yl]-5-(4-phenoxybenzylidene)thiazol-4-one
126 5-(3-benzyloxybenzylidene)-2-[4-(4-chloro-[1,2,5]thiadiazol-3-yl)piperazin-1-yl]thiazol-4-one
127 2-[4-(4-chloro-[1,2,5]thiadiazol-3-yl)piperazin-1-yl]-5-(4-hydroxy-3-methoxybenzylidene)thiazol-4-one
128 2-[4-(4-chloro-[1,2,5]thiadiazol-3-yl)piperazin-1-yl]-5-furan-2-yl-methylenethiazol-4-one
129 2-[4-(4-chloro-[1,2,5]thiadiazol-3-yl)piperazin-1-yl]-5-naphthalen-2-yl-methylenethiazol-4-one
130 3-[2-(4-benzylpiperazin-1-yl)-4-oxo-4H-thiazol-5-ylidenemethyl]benzoic acid
131 5-naphthalen-1-ylmethylene-2-piperazin-1-ylthiazol-4-one
132 2-piperazin-1-yl-5-(4-trifluoromethylbenzylidene)thiazol-4-one
133 5-(4-methylbenzylidene)-2-piperazin-1-ylthiazol-4-one
134 5-(4-hydroxy-3-methoxybenzylidene)-2-piperazin-1-ylthiazol-4-one
135 5-(4-isopropylbenzylidene)-2-morpholin-4-ylthiazol-4-one
136 5-(4-isopropylbenzylidene)-2-thiomorpholin-4-ylthiazol-4-one
137 2-[4-(2-fluorophenyl)piperazin-1-yl]-5-(4-isopropylbenzylidene)thiazol-4-one
138 5-(4-pentafluorosulfanyl)benzylidene)-2-thiomorpholin-4-ylthiazol-4-one
139 2-[4-(3-chloropyridin-2-yl)piperazin-1-yl]-5-(4-isopropylbenzylidene)thiazol-4-one
140 2-(4-benzylpiperazin-1-yl)-5-(4-butylbenzylidene)thiazol-4-one
141 2-(4-benzylpiperazin-1-yl)-5-(4-pentylbenzylidene)thiazol-4-one
142 2-(4-benzylpiperazin-1-yl)-5-(4-octylbenzylidene)thiazol-4-one
143 2-[4-(6-chloropyridin-2-yl)piperazin-1-yl]-5-(4-methylbenzylidene)thiazol-4-one
144 5-(4-tert-butylbenzylidene)-2-[4-(6-chloropyridin-2-yl)piperazin-1-yl]thiazol-4-one
145 2-[4-(6-chloropyridin-2-yl)piperazin-1-yl]-5-(4-trifluoromethylbenzylidene)thiazol-4-one
146 2-[4-(6-chloropyridin-2-yl)piperazin-1-yl]-5-(3-phenylallylidene)thiazol-4-one
147 5-[3-(4-tert-butylphenyl)allylidene]-2-[4-(6-chloropyridin-2-yl)piperazin-1-yl]thiazol-4-one
148 2-[4-(6-chloropyridin-2-yl)piperazin-1-yl]-5-(4-hydroxy-3-methoxybenzylidene)thiazol-4-one
149 2-[4-(6-chloropyridin-2-yl)piperazin-1-yl]-5-(4-isopropylbenzylidene)thiazol-4-one
150 2-[4-(6-chloropyridin-2-yl)piperazin-1-yl]-5-(4-trifluoromethoxybenzylidene)thiazol-4-one
151 2-[4-(6-chloropyridin-2-yl)piperazin-1-yl]-5-naphthalen-2-ylmethylenethiazol-4-one
152 5-(4-methylbenzylidene)-2-[4-(6-methylpyridazin-3-yl)piperazin-1-yl]thiazol-4-one
153 5-(4-isopropylbenzylidene)-2-[4-(6-methylpyridazin-3-yl)piperazin-1-yl]thiazol-4-one
154 5-(4-tert-butylbenzylidene)-2-[4-(6-methylpyridazin-3-yl)piperazin-1-yl]thiazol-4-one
155 5-[3-(4-tert-butylphenyl)allylidene]-2-[4-(6-methylpyridazin-3-yl)piperazin-1-yl]thiazol-4-one
156 2-[4-(6-methylpyridazin-3-yl)piperazin-1-yl]-5-(4-trifluoromethoxybenzylidene)thiazol-4-one
157 5-(4-hydroxy-3-methoxybenzylidene)-2-[4-(6-methylpyridazin-3-yl)piperazin-1-yl]thiazol-4-one
158 2-(4-benzylpiperazin-1-yl)-5-(4-pentafluorosulfanylbenzylidene)thiazol-4-one
159 2-(4-benzylpiperazin-1-yl)-5-{3-[4-(4-chloro-[1,2,5]thiadiazol-3-yl)piperazine-1-carbonyl]benzylidene}thiazol-4-one
160 2-[4-(6-methylpyridazin-3-yl)piperazin-1-yl]-5-(4-trifluoromethylbenzylidene)thiazol-4-one
161 2-[4-(6-methylpyridazin-3-yl)piperazin-1-yl]-5-(3-phenylallylidene)thiazol-4-one
162 2-[4-(6-methylpyridazin-3-yl)piperazin-1-yl]-5-naphthalen-2-yl-methylenethiazol-4-one
163 2-[4-(6-methylpyridazin-3-yl)piperazin-1-yl]-5-naphthalen-1-yl-methylenethiazol-4-one
164 2-(4-benzylpiperazin-1-yl)-5-(4-isopropylbenzylidene)thiazol-4-one
165 3-{2-[4-(4-chloro-[1,2,5]thiadiazol-3-yl)piperazin-1-yl]-4-oxo-4H-thiazol-5-ylidenemethyl}benzoic acid
166 5-{3-[4-(4-chloro-3-trifluoromethylphenyl)-4-hydroxypiperidine-1-carbonyl]benzylidene}-2-thiomorpholin-4-ylthiazol-4-one
167 5-{3-[4-(4-chloro-[1,2,5]thiadiazol-3-yl)piperazine-1-carbonyl]benzylidene}-2-[4-(4-chloro-[1,2,5]thiadiazol-3-yl)piperazin-1-yl]thiazol-4-one
168 5-{3-[4-(3-chloropyridin-2-yl)piperazine-1-carbonyl]benzylidene}-2-[4-(4-chloro-[1,2,5]thiadiazol-3-yl)piperazin-1-yl]thiazol-4-one
169 5-[3-(4-benzylpiperazine-1-carbonyl)benzylidene]-2-[4-(4-chloro-[1,2,5]thiadiazol-3-yl)piperazin-1-yl]thiazol-4-one
170 2-[4-(4-chloro-[1,2,5]thiadiazol-3-yl)piperazin-1-yl]-5-{3-[4-(6-methoxy-pyridin-2-yl)piperazine-1-carbonyl]benzylidene}thiazol-4-one
171 2-[4-(4-chloro-3-trifluoromethylphenyl)-4-hydroxypiperidin-1-yl]-5-(4-methylbenzylidene)thiazol-4-one
172 2-[4-(4-chloro-3-trifluoromethylphenyl)-4-hydroxypiperidin-1-yl]-5-(4-hydroxy-3-methoxybenzylidene)thiazol-4-one
173 2-[4-(4-chloro-3-trifluoromethylphenyl)-4-hydroxypiperidin-1-yl]-5-naphthalen-1-ylmethylenethiazol-4-one
174 2-[4-(4-chloro-3-trifluoromethylphenyl)-4-hydroxypiperidin-1-yl]-5-(3-methoxybenzylidene)thiazol-4-one
175 5-benzylidene-2-morpholin-4-ylthiazol-4-one
176 5-(4-methyl benzylidene)-2-morpholin-4-ylthiazol-4-one
177 2-diethylamino-5-(4-methylbenzylidene)thiazol-4-one
178 5-(4-chlorobenzylidene)-2-diethylaminothiazol-4-one
179 5-(4-isopropylbenzylidene)-2-morpholin-4-ylthiazol-4-one
180 5-benzylidene-2-diethylaminothiazol-4-one
181 5-(4-chlorobenzylidene)-2-morpholin-4-ylthiazol-4-one
182 5-(4-chlorobenzylidene)-2-piperidin-1-ylthiazol-4-one
183 5-(4-chlorobenzylidene)-2-pyrrolidin-1-ylthiazol-4-one
184 5-benzylidene-2-pyrrolidin-1-ylthiazol-4-one
185 5-benzylidene-2-pyrrolidin-1-ylthiazol-4-one
186 5-biphenyl-4-ylmethylene-2-piperidin-1-ylthiazol-4-one
187 2-azepan-1-yl-5-biphenyl-4-ylmethylenethiazol-4-one
188 5-biphenyl-4-ylmethylene-2-pyrrolidin-1-ylthiazol-4-one
189 2-morpholin-4-yl-5-(3-phenylallylidene)thiazol-4-one
190 5-(3-phenylallylidene)-2-thiomorpholin-4-ylthiazol-4-one
191 5-(3-phenylallylidene)-2-piperidin-1-ylthiazol-4-one
192 2-azepan-1-yl-5-(3-phenylallylidene)thiazol-4-one
193 5-(3-phenylallylidene)-2-pyrrolidin-1-ylthiazol-4-one
194 5-biphenyl-4-ylmethylene-2-morpholin-4-ylthiazol-4-one
195 2-thiomorpholin-4-yl-5-(3-trifluoromethylbenzylidene)thiazol-4-one
196 5-(4-chlorobenzylidene)-2-thiomorpholin-4-ylthiazol-4-one
197 5-(4-tert-butylbenzylidene)-2-morpholin-4-ylthiazol-4-one
198 5-(4-tert-butylbenzylidene)-2-thiomorpholin-4-ylthiazol-4-one
199 5-(4-tert-butylbenzylidene)-2-pyrrolidin-1-ylthiazol-4-one
200 2-azepan-1-yl-5-(4-tert-butylbenzylidene)thiazol-4-one
201 5-(4-tert-butylbenzylidene)-2-piperidin-1-ylthiazol-4-one
202 2-thiomorpholin-4-yl-5-(4-trifluoromethylbenzylidene)thiazol-4-one
203 5-(4-tert-butylbenzylidene)-2-diethylaminothiazol-4-one
204 2-morpholin-4-yl-5-(4-trifluoromethylbenzylidene)thiazol-4-one
205 2-morpholin-4-yl-5-(3-trifluoromethylbenzylidene)thiazol-4-one
206 2-morpholin-4-yl-5-(3-trifluoromethylbenzylidene)thiazol-4-one
207 5-biphenyl-4-ylmethylene-2-diethylaminothiazol-4-one
208 2-morpholin-4-yl-5-thiophen-2-ylmethylenethiazol-4-one
209 2-morpholin-4-yl-5-naphthalen-1-ylmethylenethiazol-4-one
210 2-morpholin-4-yl-5-pyridin-2-ylmethylenethiazol-4-one
211 2-morpholin-4-yl-5-(4-trifluoromethylbenzylidene)thiazol-4-one
212 2-morpholin-4-yl-5-pyridin-3-ylmethylenethiazol-4-one
213 2-morpholin-4-yl-5-pyridin-4-ylmethylenethiazol-4-one
214 2-diethylamino-5-(3-trifluoromethylbenzylidene)thiazol-4-one
215 5-(4-methylbenzylidene)-2-pyrrolidin-1-ylthiazol-4-one
216 5-(4-tert-butylbenzylidene)-2-(2-methoxyethylamino)thiazol-4-one
217 2-morpholin-4-yl-5-(4-octylbenzylidene)thiazol-4-one
218 5-(4-methylbenzylidene)-2-morpholin-4-ylthiazol-4-one
219 5-(3,4-dimethoxybenzylidene)-2-morpholin-4-ylthiazol-4-one
220 5-(2-hydroxybenzylidene)-2-morpholin-4-ylthiazol-4-one
221 5-(2-hydroxy-3-methoxybenzylidene)-2-morpholin-4-ylthiazol-4-one
222 5-(4-tert-butylbenzylidene)-2-morpholin-4-ylthiazol-4-one
223 5-(4-diethylaminobenzylidene)-2-morpholin-4-ylthiazol-4-one
224 5-(4-hydroxy-3-methoxybenzylidene)-2-morpholin-4-ylthiazol-4-one
225 4-(2-morpholin-4-yl-4-oxo-4H-thiazol-5-ylidenemethyl)benzoic acid
226 2-morpholin-4-yl-5-(2,3,4-trimethoxybenzylidene)thiazol-4-one
227 2-morpholin-4-yl-5-(3,4,5-trimethoxybenzylidene)thiazol-4-one
228 5-(4-methoxybenzylidene)-2-morpholin-4-ylthiazol-4-one
229 5-(4-hydroxybenzylidene)-2-morpholin-4-ylthiazol-4-one
230 5-(3-ethoxy-4-hydroxybenzylidene)-2-morpholin-4-ylthiazol-4-one
231 5-(3-hydroxybenzylidene)-2-morpholin-4-ylthiazol-4-one
232 5-(4-bromobenzylidene)-2-morpholin-4-ylthiazol-4-one
233 2-morpholin-4-yl-5-(4-vinyloxybenzylidene)thiazol-4-one
234 benzenesulfonic acid 4-(2-morpholin-4-yl-4-oxo-4H-thiazol-5-ylidenemethyl)phenyl ester
235 5-(4-dimethylaminobenzylidene)-2-morpholin-4-ylthiazol-4-one
236 4-(2-morpholin-4-yl-4-oxo-4H-thiazol-5-ylidenemethyl)benzoic acid methyl ester
237 5-(3-hydroxy-4-methoxybenzylidene)-2-morpholin-4-ylthiazol-4-one
238 5-[4-(3,3-dimethyl butoxy)-3-methoxybenzylidene]-2-morpholin-4-ylthiazol-4-one
239 5-(2-methoxybenzylidene)-2-morpholin-4-ylthiazol-4-one
240 5-(4-hydroxybenzylidene)-2-morpholin-4-ylthiazol-4-one
241 2-[(benzo[1,3]dioxol-5-ylmethyl)amino]-5-[3-(4-tert-butylphenyl)allylidene]thiazol-4-one
242 2-[(benzo[1,3]dioxol-5-ylmethyl)amino]-5-(4-trifluoromethylbenzylidene)thiazol-4-one
243 2-[(benzo[1,3]dioxol-5-ylmethyl)amino]-5-(4-trifluoromethoxybenzylidene)thiazol-4-one
244 2-[(benzo[1,3]dioxol-5-ylmethyl)amino]-5-naphthalen-1-ylmethylenethiazol-4-one
245 2-[(benzo[1,3]dioxol-5-ylmethyl)amino]-5-(2-chloro-3-phenylallylidene)thiazol-4-one
246 2-(4-methylbenzylamino)-5-naphthalen-1-ylmethylenethiazol-4-one
247 2-(4-methylbenzylamino)-5-(3-phenoxybenzylidene)thiazol-4-one
248 2-(4-methylbenzylamino)-5-(4-phenoxybenzylidene)thiazol-4-one
249 5-(2-chloro-3-phenylallylidene)-2-(4-methylbenzylamino)thiazol-4-one
250 2-(4-methyl benzylamino)-5-naphthalen-2-ylmethylenethiazol-4-one
251 2-(3-methoxybenzylamino)-5-(4-methylbenzylidene)thiazol-4-one
252 5-(4-tert-butylbenzylidene)-2-(3-methoxybenzylamino)thiazol-4-one
253 2-(3-methoxybenzylamino)-5-(4-trifluoromethylbenzylidene)thiazol-4-one
254 2-(3-methoxybenzylamino)-5-(4-trifluoromethoxybenzylidene)thiazol-4-one
255 2-(3-methoxybenzylamino)-5-(3-phenylallylidene)thiazol-4-one
256 5-naphthalen-1-ylmethylene-2-(4-trifluoromethylbenzylamino)thiazol-4-one
257 5-(3-phenoxybenzylidene)-2-(4-trifluoromethylbenzylamino)thiazol-4-one
258 5-(4-phenoxybenzylidene)-2-(4-trifluoromethylbenzylamino)thiazol-4-one
259 5-(2-chloro-3-phenylallylidene)-2-(4-trifluoromethylbenzylamino)thiazol-4-one
260 5-(3-benzyloxybenzylidene)-2-(4-trifluoromethylbenzylamino)thiazol-4-one
261 5-naphthalen-2-ylmethylene-2-(4-trifluoromethylbenzylamino)thiazol-4-one
262 2-(4-hydroxy-3-methoxyphenyl)-5-(4-methylbenzylidene)thiazol-4-one and
263 2-(4-tert-butylphenylamino)-5-(4-methylbenzylidene)thiazol-4-one
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates, preferably selected as appropriate from the group consisting of hydrobromides, hydrochlorides and triethylammonium salts, or in each case in the form of corresponding solvates.

Furthermore, preference may be given to the use of inventive compounds which, in the FLIPR assay, in a concentration of 10 μM, have an inhibition of the Ca²⁺ ion current in dorsal root ganglia of rats of at least 10%, preferably of at least 30%, more preferably of at least 50%, even more preferably of at least 70%, even more preferably of at least 90%, compared to the maximum achievable inhibition of the Ca²⁺ ion current with capsaicin in a concentration of 10 μM.

In the FLIPR assay, the Ca²⁺ current is quantified with the aid of a Ca²⁺-sensitive dye (Fluo-4 type, Molecular Probes Europe BV, Leiden, the Netherlands) in a fluorescent imaging plate reader (FLIPR, Molecular Devices, Sunnyvale, USA), as described below.

The present invention further provides for the use of at least one inventive 2,5-disubstituted thiazol-4-one derivative of the above-specified general formula I, in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and also, as appropriate, one or more pharmaceutically acceptable excipients, for producing a medicament for vanilloid receptor 1 (VR1/TRPV1) regulation, preferably for vanilloid receptor 1 (VR1/TRPV1) inhibition or for vanilloid receptor 1 (VR1/TRPV1) stimulation.

Preference is given to the use of at least one 2,5-disubstituted thiazol-4-one derivative of the above-specified general formula I, in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and optionally one or more pharmaceutically acceptable excipients, for producing a medicament for prophylaxis and/or treatment of disorders or diseases which are mediated at least partly by vanilloid receptors 1.

Particular preference is given to the use of at least one 2,5-disubstituted thiazol-4-one derivative of the above-specified general formula I, in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and optionally one or more pharmaceutically acceptable excipients, for producing a medicament for treatment and/or prophylaxis of pain, preferably of pain selected from the group consisting of acute pain, chronic pain, visceral pain and neuropathic pain; for prophylaxis and/or treatment of one or more disorders selected from the group consisting of migraine; depression; urine incontinence; coughing; neurodegenerative disorders, preferably selected from the group consisting of Parkinson's disease, Huntington's disease; Alzheimer's disease and multiple sclerosis; disorders of food uptake, preferably selected from the group consisting of bulimia, anorexia, obesity and cachexia; states of anxiety; cognitive dysfunctions, preferably memory impairments; cognitive deficiencies (attention deficit syndrome, ADS); epilepsy; diarrhea and pruritus; for prophylaxis and/or treatment of alcohol and/or drug and/or medicament abuse and alcohol and/or drug and/or medicament dependence, preferably for prophylaxis and/or reduction of withdrawal symptoms in the case of alcohol and/or drug and/or medicament dependence; for prophylaxis and/or reduction of evolution of tolerance to medicaments, especially medicaments based on opioids; for regulating food uptake; for modulating movement activity; for regulating the cardiovascular system; for local anesthesia; for enhancing vigilance; for enhancing libido; for diuresis and/or for antinatriuresis.

Very particular preference is given to the use of at least one 2,5-disubstituted thiazol-4-one derivative of the above-specified general formula I, in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and optionally one or more pharmaceutically acceptable excipients, for producing a medicament for treatment and/or prophylaxis of pain, preferably of pain selected from the group consisting of acute pain, chronic pain, visceral pain and neuropathic pain; for prophylaxis and/or treatment of one or more disorders selected from the group consisting of migraine; depression; neurodegenerative disorders, preferably selected from the group consisting of Parkinson's disease, Huntington's disease Alzheimer's disease and multiple sclerosis; states of anxiety; cognitive dysfunctions, preferably memory impairments; cognitive deficiencies (attention deficit syndrome, ADS); epilepsy; for prophylaxis and/or treatment of alcohol and/or drug and/or medicament abuse and alcohol and/or drug and/or medicament dependence, preferably for prophylaxis and/or reduction of withdrawal symptoms in the case of alcohol and/or drug and/or medicament dependence; for prophylaxis and/or reduction of evolution of tolerance to medicaments, especially medicaments based on opioids.

Even more particular preference is given to the use of at least one 2,5-disubstituted thiazol-4-one derivative of the above-specified general formula I, in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and optionally one or more pharmaceutically acceptable excipients, for producing a medicament for treatment and/or prophylaxis of pain, preferably of pain selected from the group consisting of acute pain, chronic pain, visceral pain and neuropathic pain.

The present invention further provides 2,5-disubstituted thiazol-4-one derivatives of the general formula Ia

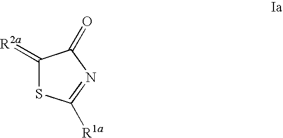

in which
R^{1a} is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   is an NR^{3a}R^{4a} group;
   is an NR^{5a}—C(═O)—R^{6a} group;
   or is an NR^{7a}—C(═O)—NR^{8a}R^{9a} group;
R^{2a} is CH—CH═C(CH₃)—W^{a}-Z^{a}, CH—CH═CH—W^{a}-Z^{a}, CH—C(CH₃)═CH—W^{a}-Z^{a}, CH—C(phenyl)=CH—W^{a}-Z^{a}, CH—CBr═CH—W^{a}-Z^{a}, CH—CCl═CH—W^{a}-Z^{a}, CH—CF═CH—W^{a}-Z^{a}, CH—C(OH)═CH—W^{a}-Z^{a}, CH—CH₂—CH₂—W^{a}-Z^{a}, CH—CH₂—CH(CH₃)_W^{a}-Z^{a} or CH—CH(CH₃)—CH₂—W^{a}-Z^{a};
   where W^{a} may in each case be absent or is in each case a radical selected from the group consisting of O, S, N(H), N(CH₃), N(C₂H₅) and N[CH(CH₃)₂];
R^{3a} is a linear or branched, saturated or unsaturated, unsubstituted or at least monosubstituted aliphatic radical optionally having heteroatom as a chain member;
   is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   or is an unsubstituted or at least monosubstituted aryl or heteroaryl radical which may be fused to an unsubstituted or at least monosubstituted mono- or polycyclic ring system and/or may be bonded via a linear or branched, unsubstituted or at least monosubstituted alkylene, alkenylene or alkynylene group;
R^{4a} is a hydrogen radical
   or is a linear or branched, saturated or unsaturated, unsubstituted or at least monosubstituted aliphatic radical optionally having at least one heteroatom as a chain member;
R^{5a}, R^{7a} and R^{9a} are each independently
   a hydrogen radical
   or a linear or branched, saturated or unsaturated, unsubstituted or at least monosubstituted aliphatic radical;
R^{6a} and R^{8a} are each independently
   an unsubstituted or at least monosubstituted aryl or heteroaryl radical which may be fused to an unsubstituted or at least monosubstituted mono- or polycyclic ring system;
Z^{a} is a linear or branched, saturated or unsaturated, unsubstituted or at least monosubstituted aliphatic radical;
   is an aryl radical which may be fused to an unsubstituted or at least monosubstituted mono- or polycyclic ring system and/or may be substituted by identical or different substituents R^{12a};
   or is an heteroaryl radical which may be fused to an unsubstituted or at least monosubstituted mono- or polycyclic ring system and/or may be substituted by identical or different substituents R^{12a};
R^{12a} is a radical selected from the group consisting of F, Cl, Br, I, —CN, —CF₃, —SF₅, —OH, —O—C₁₋₁₀-alkyl, —O—C₂₋₁₀-alkenyl, —NH₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₁₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)— benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted;
   is a —C(═O)—NR^{13a}—(CH₂)ₘ—NR^{14a}—R^{15a} group where m is 0, 1, 2, 3, 4 or 5;
   or is a —C(═O)—R^{16a} group;
R^{13a} and R^{14a} are each independently
   a hydrogen radical
   or a linear or branched, saturated or unsaturated, unsubstituted or at least monosubstituted aliphatic radical;
R^{15a} is an unsubstituted or at least monosubstituted aryl or heteroaryl radical which may be fused to an unsubstituted or at least monosubstituted mono- or polycyclic ring system; and
R^{16a} is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates.

Particular preference is given to 2,5-disubstituted thiazol-4-one derivatives of the above-specified general formula Ia in which
R^{1a} is one of the following radicals

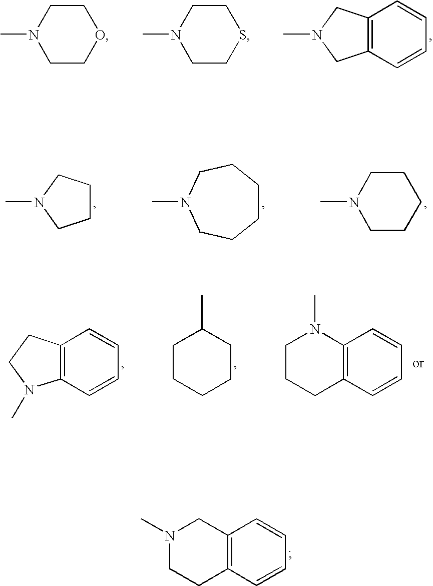

where the radical may be substituted by 1, 2 or 3 substituents selected independently from the group consisting of —OH, —O—CH₃, —O—C₂H₅, methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl and phenyl; where the cyclic moiety of the phenyl radical may in each case be substituted by 1, 2 or 3 substituents selected independently from the group consisting of F, Cl, Br and —CF₃;
is one of the following radicals

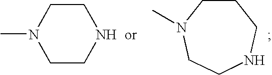

where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is an NR^{3a}R^{4a} group
or is an NR^{7a}—C(═O)—NR^{8a}R^{9a} group;
R^{2a} is CH—CH═C(CH₃)-Z^{a}, CH—CH═CH-Z^{a}, CH—CH═CH—S-Z^{a}, CH—CH═CH—O-Z^{a}, CH—CH═CH—N(CH₃)-Z^{a}, CH—C(CH₃)═CH-Z^{a}, CH—C(phenyl)═CH-Z^{a}, CH—CBr═CH-Z^{a}, CH—CCl═CH-Z^{a}, CH—CF═CH-Z^{a}, CH—C(OH)═CH-Z^{a}, CH—CH₂—CH₂-Z^{a}, CH—CH₂—CH(CH₃)-Z^{a} or CH—CH(CH₃)—CH₂-Z^{a};
R^{3a} is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
   is a radical selected from the group consisting of cyclopentyl, cyclohexyl, cycloheptyl, cyclopentenyl, cyclohexenyl and cycloheptenyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)— phenyl, —N(C₂H₅)-phenyl, —N[CH(CH₃)₂]-phenyl, —N(CH₃)-pyridinyl, —N(C₂H₅)— pyridinyl, —N [CH(CH₃)₂]-pyridinyl;
   or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R^{4a} is a hydrogen radical
   or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{7a} and R^{8a} are each independently
   a hydrogen radical
   or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{9a} is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CH₃, —O—C₂H₅, —O—CF₃, —S—CF₃, methyl, ethyl, n-propyl, isopropyl, n-butyl, sec-butyl, isobutyl and tert-butyl;
Z^{a} is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl;
   is a radical selected from the group consisting of phenyl, naphthyl, phenanthrenyl, (1,3)-benzodioxolyl and (1,4)-benzodioxanyl, which is optionally substituted by 1, 2, 3, 4 or 5 identical or different substituents R^{12a};
   or is a radical selected from the group consisting of thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R^{12a};
R^{12a} is a radical selected from the group consisting of F, Cl, Br, —SF₅, —CF₃, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, methyl, ethyl, n-propyl, isopropyl, n-butyl, sec-butyl, tert-butyl, n-pentyl, n-hexyl, n-heptyl and n-octyl;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates.

Very particular preference is given to 2,5-disubstituted thiazol-4-one derivatives of the above-specified general formula Ia in which
R^{1a} is one of the following radicals

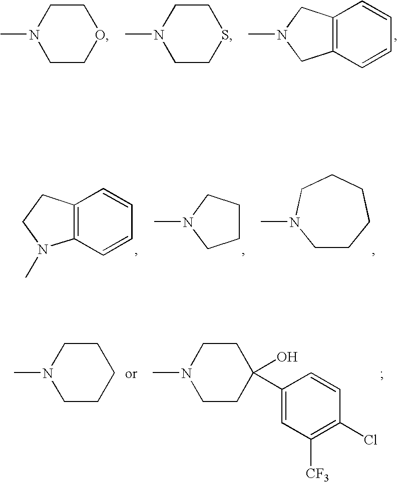

is the following radical

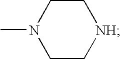

where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is an NR^{3a}R^{4a} group
or is an NR^{7a}—C(═O)—NR^{8a}R^{9a} group;
R^{2a} is CH—CH═C(CH₃)-Z^{a}, CH—CH═CH-Z^{a}, CH—CH═CH—S-Z^{a}, CH—C(CH₃)═CH-Z^{a}, CH—C(phenyl)=CH-Z^{a}, CH—CBr═CH-Z^{a}, CH—CCl═CH-Z^{a}, CH—CF═CH-Z^{a}, CH—CH₂—CH₂-Z^{a}, CH—CH₂—CH(CH₃)-Z^{a} or CH—CH(CH₃)—CH₂-Z^{a};
R^{3a} is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethyl-propyl, n-pentyl, sec-pentyl, n-hexyl and n-heptyl;
   or is a radical selected from the group consisting of phenyl, naphthyl, benzyl, (1,3)-benzodioxolyl and (1,4)-benzodioxanyl; where the radical may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R^{4a} is a hydrogen radical
   or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{7a} and R^{8a} are each a hydrogen radical;
R^{9a} is a phenyl radical which may be substituted by 1, 2, 3, 4 or 5 substituents selected independently from the group consisting of F, Cl, Br and —CF₃;
Z^{a} is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl and n-octyl;
   or is a phenyl or naphthyl radical which may be substituted by 1, 2, 3, 4 or 5 identical or different substituents R^{12a};
R^{12a} is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl;
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, or in each case in the form of corresponding solvates.

Even more preferred are 2,5-disubstituted thiazol-4-one derivatives of the above-specified general formula Ia selected from the group consisting of
1. 5-[3-(4-methoxyphenyl)but-2-enylidene]-2-morpholin-4-ylthiazol-4-one
2. 2-morpholin-4-yl-5-(3-phenylbutylidene)thiazol-4-one
3. 2-morpholin-4-yl-5-(3-phenylpropylidene)thiazol-4-one
4. 5-[3-(4-fluorophenyl)allylidene]-2-morpholin-4-ylthiazol-4-one
5. 2-morpholin-4-yl-5-(3-m-tolylallylidene)thiazol-4-one
6. 2-morpholin-4-yl-5-(3-phenylsulfanylallylidene)thiazol-4-one
7. 2-morpholin-4-yl-5-(3-p-tolylallylidene)thiazol-4-one
8. 5-(3-phenylallylidene)-2-thiomorpholin-4-ylthiazol-4-one
9. 2-morpholin-4-yl-5-octylidenethiazol-4-one
10. 5-butylidene-2-morpholin-4-ylthiazol-4-one
11. 2-[4-(3-chloropyridin-2-yl)piperazin-1-yl]-5-(3-phenylallylidene)thiazol-4-one
12. 5-hexylidene-2-morpholin-4-ylthiazol-4-one
13. 2-morpholin-4-yl-5-(3-p-tolylbut-2-enylidene)thiazol-4-one
14. 5-[3-(4-tert-butylphenyl)but-2-enylidene]-2-morpholin-4-ylthiazol-4-one
15. 2-[4-(3-methoxyphenyl)piperazin-1-yl]-5-(3-phenylallylidene)thiazol-4-one
16. 5-[3-(4-tert-butylphenyl)allylidene]-2-[4-(3-methoxyphenyl)piperazin-1-yl]-thiazol-4-one
17. 5-[3-(4-hydroxy-3-methoxyphenyl)allylidene]-2-thiomorpholin-4-ylthiazol-4-one
18. 2-morpholin-4-yl-5-[3-(4-trifluoromethylphenyl)but-2-enylidene]thiazol-4-one
19. 2-morpholin-4-yl-5-[3-(3-trifluoromethylphenyl)but-2-enylidene]thiazol-4-one
20. 5-(2,3-diphenylallylidene)-2-morpholin-4-ylthiazol-4-one
21. 2-[4-(4-methoxyphenyl)piperazin-1-yl]-5-(3-phenylallylidene)thiazol-4-one
22. 2-[4-(2-chlorophenyl)piperazin-1-yl]-5-(3-phenylallylidene)thiazol-4-one
23. 5-(3-phenylallylidene)-2-(4-phenylpiperazin-1-yl)thiazol-4-one
24. 5-decylidene-2-morpholin-4-ylthiazol-4-one
25. 5-[3-(4-tert-butylphenyl)allylidene]-2-morpholin-4-ylthiazol-4-one
26. 5-(2-bromo-3-phenylalylidene)-2-[4-(3-chloropyridin-2-yl)piperazin-1-yl]thiazol-4-one
27. 5-[3-(4-tert-butylphenyl)allylidene]-2-[4-(3-chloropyridin-2-yl)piperazin-1-yl]-thiazol-4-one
28. 2-[4-(3-chloropyridin-2-yl)piperazin-1-yl]-5-(2-methyl-3-phenylallylidene)thiazol-4-one
29. 5-(2-chloro-3-phenylallylidene)-2-[4-(3-chloropyridin-2-yl)piperazin-1-yl]thiazol-4-one
30. 2-[4-(2-fluorophenyl)piperazin-1-yl]-5-(3-phenylallylidene)thiazol-4-one
31. 5-[3-(4-tert-butyl phenyl)allylidene]-2-[4-(2-fluorophenyl)piperazin-1-yl]thiazol-4-one
32. 5-(2-chloro-3-phenylallylidene)-2-[4-(2-fluorophenyl)piperazin-1-yl]thiazol-4-one
33. 5-(2-chloro-3-phenylallylidene)-2-thiomorpholin-4-ylthiazol-4-one
34. 1-[4-oxo-5-(3-phenylallylidene)-4,5-dihydrothiazol-2-yl]-3-(2-trifluoromethylphenyl)urea
35. 5-[3-(4-tert-butylphenyl)allylidene]-2-[4-(4-chloro-[1,2,5]thiadiazol-3-yl)-piperazin-1-yl]thiazol-4-one
36. 5-[3-(4-tert-butylphenyl)allylidene]-2-[4-(6-chloropyridin-2-yl)piperazin-1-yl]-thiazol-4-one
37. 5-[3-(4-tert-butylphenyl)allylidene]-2-[4-(6-methylpyridazin-3-yl)-piperazin-1-yl]-thiazol-4-one
38. 2-[4-(6-methyl-pyridazin-3-yl)piperazin-1-yl]-5-(3-phenylallylidene)thiazol-4-one
39. 5-(3-phenylallylidene)-2-thiomorpholin-4-ylthiazol-4-one
40. 5-(3-phenylallylidene)-2-piperidin-1-ylthiazol-4-one
41. 2-azepan-1-yl-5-(3-phenylallylidene)thiazol-4-one
42. 5-(3-phenylallylidene)-2-pyrrolidin-1-ylthiazol-4-one
43. 5-(2-chloro-3-phenylallylidene)-2-morpholin-4-ylthiazol-4-one
44. 2-[(benzo[1,3]dioxol-5-ylmethyl)amino]-5-[3-(4-tert-butylphenyl)-allylidene]thiazol-4-one
45. 2-(3-methoxybenzylamino)-5-(3-phenylallylidene)thiazol-4-one
46. 5-(2-chloro-3-phenylallylidene)-2-(4-trifluoromethylbenzylamino)thiazol-4-one
   in each case, as appropriate, in the form of one of its pure stereoisomers, especially enantiomers or diastereomers, of its racemates or in the form of a mixture of stereoisomers, especially of the enantiomers and/or diastereomers, in any mixing ratio, or in each case in the form of corresponding salts, preferably its hydrochlorides or hydrobromides, or in each case in the form of corresponding solvates.

In addition, preference may be given to inventive compounds which, in the FLIPR assay, in a concentration of 10 μM, have an inhibition of the Ca²⁺ ion current in dorsal root ganglia of rats of at least 10%, preferably of at least 30%, more preferably of at least 50%, even more preferably of at least 70%, even more preferably of at least 90%, compared to the maximum achievable inhibition of the Ca²⁺ ion current with capsaicin in a concentration of 10 μM.

In the FLIPR assay, the Ca²⁺ current is quantified with the aid of a Ca²⁺-sensitive dye (Fluo-4, Molecular Probes Europe BV, Leiden, the Netherlands) in a fluorescent imaging plate reader (FLIPR, Molecular Devices, Sunnyvale, USA), as described below.

The present invention further provides a process for preparing inventive compounds of the above-specified general formula I, according to which 2-aminothiazol-4-one is reacted in acetic acid in the presence of at least one salt selected from the group consisting of sodium acetate and potassium acetate or in a reaction medium selected from the group consisting of methanol, ethanol, isopropanol and n-butanol in the presence of at least one organic base selected from the group consisting of triethylamine, pyridine, diisopropylamine and N-methylmorpholine with at least one compound of the general formula R—C(═O)—H, in which R is —CH═C(CH₃)—W^{a}-Z^{a}, —CH═CH_W^{a}-Z^{a}, —C(CH₃)═CH—W^{a}-Z^{a}, —C(phenyl)=CH—W^{a}-Z^{a}, —CBr═CH—W^{a}-Z^{a}, —CCl═CH_W^{a}-Z^{a}, —CF═CH—W^{a}-Z^{a}, —C(OH)═CH_W^{a}-Z^{a}, —CH₂—CH₂—W^{a}-Z^{a}, —CH₂—CH(CH₃)_W^{a}-Z^{a} or —CH(CH₃)—CH₂—W^{a}-Z^{a}, where W^{a} and Z^{a} are each as defined above, to give at least one compound of the general formula IIa,

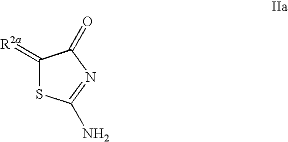

where R^{2a} is as defined above, and the latter is optionally purified and/or isolated,
and at least one compound of medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{3a} or of the general formula LG-R^{7a} or of the general formula LG-R^{5a}, in which LG is in each case a leaving group, preferably a halogen atom, more preferably a chlorine atom, and R^{3a}, R^{5a} and R^{7a} are each as defined above to give at least one compound of the general formula Ia in which R^{2a} is as defined above and R^{1a} is an NHR^{3a}—, NHR^{5a}— or NHR^{7a} group, and the latter is optionally purified and/or isolated,
and optionally at least one compound of the general formula IIa in which R^{2a} is as defined above and R^{1a} is an NHR^{7a} group is reacted in a reaction medium with at least one isocyanate of the general formula R^{8a}—N═C═O, in which R^{8a} is as defined above, optionally in the presence of at least one base, preferably in the presence of at least one base selected from the group consisting of triethylamine, 4,4-dimethylaminopyridine and diisopropylethylamine, to give at least one compound of the general formula Ia in which R^{2a} is as defined above and R^{1a} is an NR^{7a}—C(═O)_NHR^{8a} group, and the latter is optionally purified and/or isolated;
and optionally at least one compound of the general formula Ia in which R^{2a} is as defined above and R^{1a} is an NR^{7a}—C(═O)—NHR^{8a} group is reacted in a reaction medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{9a} in which LG is a leaving group, preferably a halogen atom, more preferably a chlorine atom, and R^{9a} is as defined above to give at least one compound of the general formula Ia in which R^{2a} is as defined above and R^{1a} is an NR^{7a}—C(═O)—NR^{8a}R^{9a} group, and the latter is optionally purified and/or isolated; or
optionally at least one compound of the general formula IIa in which R^{2a} is as defined above and R^{1a} is an NHR^{5a} group is reacted in a reaction medium, optionally in the presence of at least one base, with at least one compound of the general formula R^{6a}—C(═O)-LG in which R^{6a} is as defined above and LG is a leaving group, preferably a halogen radical, or in a reaction medium in the presence of at least one coupling reagent, optionally in the presence of at least one base, with a compound of the general formula R^{6a}—C(═O)—OH in which R^{6a} is as defined above, to give at least one compound of the general formula Ia in which R^{2a} is as defined above and R^{1a} is an NR^{5a}—C(═O)—R^{6a} group, and the latter is optionally purified and/or isolated or
optionally at least one compound of the general formula Ia in which R^{2a} is as defined above and R^{1a} is an NHR^{3a} group is reacted in a reaction medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{4a} in which LG is a leaving group, preferably a halogen atom, more preferably a chlorine atom, and R^{4a} is as defined above to give at least one compound of the general formula Ia in which R^{2a} is as defined above and R^{1a} is an NR^{3a}R^{4a} group, and the latter is optionally purified and/or isolated.

The reaction of compounds of the general formula IIa in which R^{1a} is an —NHR^{5a} group with compounds of the general formula R^{6a}—C(═O)—OH is effected preferably in a reaction medium selected from the group consisting of diethyl ether, tetrahydrofuran, acetonitrile, methanol, ethanol, dimethylformamide, dichloromethane and corresponding mixtures, optionally in the presence of an organic base, preferably selected from the group consisting of triethylamine, 4,4-dimethylaminopyridine, pyridine and diisopropylethylamine, or of an inorganic base, at temperatures of preferably from −70° C. to 100° C.

The reaction of compounds of the general formula IIa in which R^{1a} is an —NHR^{5a} group with compounds of the general formula R^{6a}—C(═O)—OH is effected preferably in a reaction medium selected from the group consisting of diethyl ether, tetrahydrofuran, acetonitrile, methanol, ethanol, dimethylformamide, dichloromethane and corresponding mixtures, optionally in the presence of at least one coupling reagent, preferably selected from the group consisting of 1-benzotriazolyloxytris(dimethylamino)phosphonium hexafluorophosphate (BOP), dicyclohexylcarbodiimide (DCC), N′-(3-dimethylaminopropyl)-N-ethylcarbodiimide (EDCI), N-[(dimethylamino)-1H-1,2,3-triazolo[4,5-b]pyridino-1-ylmethylene]-N-methylmethanaminium hexafluorophosphate N-oxide (HATU), O-(benzotriazol-1-yl)-N,N, N′,N′-tetramethyluronium hexafluorophosphate (HBTU), O-(benzotriazol-1-yl)-N,N,N′,N′-tetramethyluronium tetrafluoroborate (TBTU), 1-hydroxybenzotriazole (HOBt) and 1-hydroxy-7-azabenzotriazole (HOAt), optionally in the presence of at least one inorganic base, preferably selected from the group consisting of potassium carbonate and cesium carbonate, or of an organic base, preferably selected from the group consisting of triethylamine, 4-methylmorpholine, pyridine, N,N-dimethylaminopyridine and diisopropylethylamine, preferably at temperatures of from −70° C. to 100° C.

Likewise provided by the present invention is a process for preparing 2,5-disubstituted thiazol-4-one derivatives of the general formula Ia, according to which at least one compound of the general formula R^{1a}—CN in which R^{1a} is as defined above with the exception of an NR^{3a}R^{4a} group, of an NR^{5a}—C(═O)—R^{6a} group and of an NR^{7a}—C(═O)—NR^{8a}R^{9a} group, is reacted in a reaction medium selected from the group consisting of methanol, ethanol, isopropanol and n-butanol, in the presence of at least one organic base selected from the group consisting of triethylamine, pyridine, diisopropylamine and N-methylmorpholine or in pyridine as a reaction medium, with thioglycolic acid to give at least one compound of the general formula IIIa

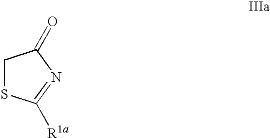

in which R^{1a} is as defined above with the exception of an NR^{3a}R^{4a} group, of an NR^{5a}—C(═O)—R^{6a} group and of an NR^{7a}—C(═O)—NR^{8a}R^{9a} group, and the latter is optionally purified and/or isolated;
or at least one compound of the general formula H₂N—C(═S)—NHR^{1a} in which R^{1a} is as defined above with the exception of an NR^{3a}R^{4a} group, of an NR^{5a}—C(═O)—R^{6a} group and of an NR^{7a}—C(═O)—NR^{8a}R^{9a} group, is reacted in a reaction medium selected from the group consisting of methanol, ethanol, isopropanol and n-butanol, optionally in the presence of at least one organic base selected from the group consisting of triethylamine, pyridine, diisopropylamine and N-methylmorpholine, with chloroacetic acid to give at least one compound of the general formula IIIa in which R^{1a} is as defined above with the exception of an NR^{3a}R^{4a} group, of an NR^{5a}—C(═O)—R^{6a} group and of an NR^{7a}—C(═O)—NR^{8a}R^{9a} group, and the latter is optionally purified and/or isolated;
and at least one compound of the general formula IIIa is reacted in acetic acid in the presence of at least one salt selected from the group consisting of sodium acetate and potassium acetate or in a reaction medium selected from the group consisting of methanol, ethanol, isopropanol and n-butanol in the presence of at least one organic base selected from the group consisting of triethylamine, pyridine, diisopropylamine and N-methylmorpholine with at least one compound of the general formula R—C(═O)—H in which R is —CH═C(CH₃)—W^{a}-Z^{a}, —CH═CH—W^{a}-Z^{a}, —C(CH₃)═CH—W^{a}-Z^{a}, —C(phenyl)=CH—W^{a}-Z^{a}, —CBr═CH—W^{a}-Z^{a}, —CCl═CH_W^{a}-Z^{a}, —CF═CH_W^{a}-Z^{a}, C(OH)═CH_W^{a}-Z^{a}, —CH₂—CH₂—W^{a}-Z^{a}, —CH₂—CH(CH₃)—W^{a}-Z^{a} or —CH(CH₃)—CH₂—W^{a}-Z^{a}, where W^{a} and Z^{a} are each as defined above, to give at least one compound of the general formula Ia in which R^{1a} is as defined above with the exception of an NR^{3a}R^{4a} group, of an NR^{5a}—C(═O)—R^{6a} group and of an NR^{7a}—C(═O)—NR^{8a}R^{9a} group and R^{2a} is as defined above.

The compounds of the above-specified general formulae R^{1a}—CN, R—C(═O)—H, R^{3a}-LG, R^{4a}-LG, R^{5a}-LG, R^{7a}-LG, R^{9a}-LG, H₂N—C(═S)—NHR^{1a}, R^{6a}—C(═O)-LG, R^{6a}—C(═O)—OH and R^{8a}—N═C═O are each commercially available on the market and can also be prepared by customary processes known to those skilled in the art.

Compounds of the above-specified general formula H₂N—C(═S)—NHR^{1a} can preferably be obtained by reacting 1,1′-thiocarbonyldiimidazole with compounds of the general formula R^{1a}NH₂ in tetrahydrofuran.

The above-described reactions may each be carried out under the customary conditions familiar to those skilled in the art, for example with regard to pressure or sequence of addition of the components. Optionally, the process regime which is optimal under the particular conditions can be determined by the person skilled in the art by simple preliminary experiments. The intermediates and end products obtained by the above-described reactions may in each case, if desired and/or necessary, be purified and/or isolated by customary methods known to those skilled in the art. Suitable purification processes are, for example, extraction processes and chromatographic processes such as column chromatography or preparative chromatography. All of the aforementioned process steps, and in each case also the purification and/or isolation of intermediates or end products, can be performed partly or completely under an inert gas atmosphere, preferably under a nitrogen atmosphere.

The inventive 2,5-disubstituted thiazol-4-one derivatives of the aforementioned general formulae I and Ia, referred to hereinafter only as 2,5-disubstituted thiazol-4-one derivatives of the general formula I, and corresponding stereoisomers, may be obtained in the form of their free bases, of their free acids or else in the form of corresponding salts, especially physiologically compatible salts. The free bases of the particular inventive 2,5-disubstituted thiazol-4-one derivatives of the aforementioned general formula I and corresponding stereoisomers may, for example, be converted to the corresponding salts, preferably physiologically compatible salts, by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid. The free bases of the particular 2,5-disubstituted thiazol-4-one derivatives of the aforementioned general formula I and corresponding stereoisomers can likewise be converted to the corresponding physiologically compatible salts with the free acid or a salt of a sugar substitute, for example saccharin, cyclamate or acesulfame.

Accordingly, the free acids of the 2,5-disubstituted thiazol-4-one derivatives of the aforementioned general formula I and corresponding stereoisomers can be converted to the corresponding physiologically compatible salts by reaction with a suitable base. Examples include the alkali metal salts, alkaline earth metal salts or ammonium salts [NHₓR₄₋ₓ]⁺ in which x=0, 1, 2, 3 or 4 and R is a linear or branched C₁₋₄-alkyl radical.

The inventive 2,5-disubstituted thiazol-4-one derivatives of the aforementioned general formula I and corresponding stereoisomers may optionally, just like the corresponding acids, the corresponding bases or salts of these compounds, also be obtained in the form of their solvates, preferably in the form of their hydrates, by customary methods known to those skilled in the art.

When the inventive 2,5-disubstituted thiazol-4-one derivatives of the aforementioned general formula I, after they have been prepared, are obtained in the form of a mixture of their stereoisomers, preferably in the form of their racemates or other mixtures of their different enantiomers and/or diastereomers, they can be separated and optionally isolated by customary processes known to those skilled in the art. Examples include chromatographic separation processes, especially liquid chromatography processes under standard pressure or under elevated pressure, preferably MPLC and HPLC processes, and processes for fractional crystallization. It is possible especially to separate individual enantiomers from one another, for example by means of HPLC on a chiral stationary phase, or, by means of crystallization of diastereomeric salts formed with chiral acids, for instance (+)-tartaric acid, (−)-tartaric acid or (+)-10-camphorsulfonic acid.

The inventive 2,5-disubstituted thiazol-4-one derivatives of the aforementioned general formula I and corresponding stereoisomers, and also in each case the corresponding acids, bases, salts and solvates, are toxicologically safe and are therefore suitable as active pharmaceutical ingredients in medicaments.

The present invention therefore further provides a medicament comprising at least one inventive 2,5-disubstituted thiazol-4-one derivative of the above-specified general formula I, in each case optionally in the form of its pure stereoisomers, especially enantiomers or diastereomers, of its racemates or in the form of a mixture of stereoisomers, especially of the enantiomers and/or diastereomers, in any mixing ratio, or in each case in the form of a corresponding salt, or in each case in the form of a corresponding solvate, and optionally one or more pharmaceutically compatible excipients.

These inventive medicaments are suitable especially for vanilloid receptor 1 (VR1/TRPV1) regulation, especially for vanilloid receptor 1 (VR1/TRPV1) inhibition or for vanilloid receptor 1 stimulation.

The inventive medicaments are likewise suitable with preference for prophylaxis and/or treatment of disorders or diseases which are mediated at least partly by vanilloid receptors 1.

The inventive medicament is therefore particularly suitable for treatment and/or prophylaxis of pain, preferably of pain selected from the group consisting of acute pain, chronic pain, visceral pain and neuropathic pain; for prophylaxis and/or treatment of one or more disorders selected from the group consisting of migraine; depression; urine incontinence; coughing; neurodegenerative disorders, preferably selected from the group consisting of Parkinson's disease, Huntington's disease; Alzheimer's disease and multiple sclerosis; disorders of food uptake, preferably selected from the group consisting of bulimia, anorexia, obesity and cachexia; states of anxiety; cognitive dysfunctions, preferably memory impairments; cognitive deficiencies (attention deficit syndrome, ADS); epilepsy; diarrhea and pruritus; for prophylaxis and/or treatment of alcohol and/or drug and/or medicament abuse and alcohol and/or drug and/or medicament dependence, preferably for prophylaxis and/or reduction of withdrawal symptoms in the case of alcohol and/or drug and/or medicament dependence; for prophylaxis and/or reduction of evolution of tolerance to medicaments, especially medicaments based on opioids; for regulating food uptake; for modulating movement activity; for regulating the cardiovascular system; for local anesthesia; for enhancing vigilance, for enhancing libido; for diuresis and/or for antinatriuresis.

The inventive medicament is therefore particularly preferably suitable for treatment and/or prophylaxis of pain, preferably of pain selected from the group consisting of acute pain, chronic pain, visceral pain and neuropathic pain; for prophylaxis and/or treatment of one or more disorders selected from the group consisting of migraine; depression; neurodegenerative disorders, preferably selected from the group consisting of Parkinson's disease, Huntington's disease, Alzheimer's disease and multiple sclerosis; states of anxiety; cognitive dysfunctions, preferably memory impairments; cognitive deficiencies (attention deficit syndrome, ADS); epilepsy; for prophylaxis and/or treatment of alcohol and/or drug and/or medicament abuse and alcohol and/or drug and/or medicament dependence, preferably for prophylaxis and/or reduction of withdrawal symptoms in the case of alcohol and/or drug and/or medicament dependence; for prophylaxis and/or reduction of evolution of tolerance to medicaments, especially medicaments based on opioids.

The inventive medicament is very particularly suitable for treatment and/or prophylaxis of pain, preferably of pain selected from the group consisting of acute pain, chronic pain, visceral pain and neuropathic pain.

The inventive medicament is suitable for administration to adults and children, including infants and babies. The inventive medicament may be present as a liquid, semisolid or solid medicament form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, optionally compressed to tablets, filled into capsules or suspended in a liquid, and may also be administered as such.

In addition to at least one 2,5-disubstituted thiazol-4-one derivative of the above-specified general formula I, optionally in the form of its pure stereoisomers, especially enantiomers or diastereomers, of its racemates or in the form of mixtures of the stereoisomers, especially of the enantiomers or diastereomers, in any mixing ratio, or optionally in the form of a corresponding salt or in each case in the form of a corresponding solvate, the inventive medicament typically comprises further physiologically compatible pharmaceutical assistants which may, for example, be selected from the group consisting of carrier materials, fillers, solvents, diluents, surfactants, dyes, preservatives, disintegrants, lubricants, aromas and binders.

The selection of the physiologically compatible assistants and the amounts thereof to be used depends upon whether the medicament is to be administered orally, subcutaneously, parenterally, intravenously, intraperitoneally, intradermally, intramuscularly, intranasally, buccally, rectally or locally, for example to infections on the skin, the mucous membranes and in the eyes. For oral application, suitable formulations are preferably those in the form of tablets, coated tablets, capsules, granules, pellets, drops, juices and syrups; suitable formulations for parenteral, topical and inhalative application are preferably solutions, suspensions, readily reconstitutable dry formulations and sprays.

The inventive 2,5-disubstituted thiazol-4-one derivatives used in the inventive medicament, in a depot, in dissolved form or in a plaster, optionally with addition of skin penetration-promoting agents, are suitable percutaneous administration formulations. Orally or percutaneously applicable formulation forms may also release the particular inventive 2,5-disubstituted thiazol-4-one derivative in a retarded manner.

The inventive medicaments are produced with the aid of conventional means, apparatus, methods and processes known from the prior art, as described, for example, in “Remington's Pharmaceutical Sciences”, editor A. R. Gennaro, 17th edition, Mack Publishing Company, Easton, Pa., 1985, more particularly in part 8, chapter 76 to 93. The corresponding description is hereby included as a reference and forms part of the disclosure.

The amount of the particular inventive 2,5-disubstituted thiazol-4-one derivatives of the above-specified general formula I to be administered to the patient may vary and is, for example, dependent on the weight or age of the patient and of the administration method, the indication and the severity of the disorder. Typically, from 0.001 to 100 mg/kg, preferably from 0.05 to 75 mg/kg, more preferably from 0.05 to 50 mg/kg, of body weight of the patient of at least one such inventive compound are administered.

### Pharmacological Methods:

### I. Functional Study on the Vanilloid Receptor 1 (VR1/TRPV1 Receptor)

The agonistic or antagonistic action of the substances to be studied on the vanilloid receptor 1 (VR1/TRPV1) of the rat species can be determined with the following assay. In this assay, the Ca²⁺ current through the receptor channel is quantified with the aid of a Ca²⁺-sensitive dye (Fluo-4 type, Molecular Probes Europe BV, Leiden the Netherlands) in a fluorescent imaging plate reader (FLIPR, Molecular Devices, Sunnyvale, USA).

### Method:

Complete medium: 50 ml HAMS F12 Nutrient Mixture (Gibco Invitrogen GmbH, Karlsruhe, Germany) with
10% by volume of FCS (fetal calf serum, Gibco Invitrogen GmbH, Karlsruhe, Germany, heat-inactivated);

### 2 mM L-glutamine (Sigma, Munich, Germany);

1% by weight of AA solution (antibiotic/antimycotic solution, PAA, Pasching, Austria) and 25 ng/ml of NGF medium (2.5 S, Gibco Invitrogen GmbH, Karlsruhe, Germany)

Cell culture plate: poly-D-lysine-coated, black 96-hole plates with a clear bottom (96 well black/clear plate, BD Biosciences, Heidelberg, Germany) are additionally coated with laminin (Gibco Invitrogen GmbH, Karlsruhe, Germany) by diluting laminin to a concentration of 100 μg/ml with PBS (Ca—Mg-free PBS, Gibco Invitrogen GmbH, Karlsruhe, Germany). Aliquots having a concentration of 100 μg/ml of laminin are withdrawn and stored at −20° C. The aliquots are diluted with PBS in a ratio of 1:10 to 10 μg/ml of laminin and in each case 50 μl of the solution are pipetted into a well of the cell culture plate. The cell culture plates are incubated at 37° C. for at least two hours, the supernatant solution is removed by suction and the wells are each washed twice with PBS. The coated cell culture plates are stored with supernatant PBS which is not removed until directly before the application of the cells.

### Preparation of the Cells:

The spinal column is removed from beheaded rats and placed directly into cold HBSS buffer (Hank's buffered saline solution, Gibco Invitrogen GmbH, Karlsruhe, Germany), i.e. placed in an ice bath, which has been admixed with 1% by volume of an AA solution (antibiotic/antimycotic solution, PAA, Pasching, Austria). The spinal column is severed longitudinally and removed from the spinal canal together with fasciae. Subsequently, the dorsal root ganglia (DRGs) are removed and in turn stored in cold HBSS buffer admixed with 1% by volume of an AA solution. The DRGs freed completely of blood residues and spinal nerves are in each case transferred to 500 μl of cold type 2 collagenase (PAA, Pasching, Austria) and incubated at 37° C. for 35 minutes. After addition of 2.5% by volume of trypsin (PAA, Pasching, Austria), incubation is continued at 37° C. for a further 10 minutes. After complete incubation, the enzyme solution is cautiously pipetted off and the remaining DRGs are each suspended with 500 μl of complete medium.

The DRGs are each suspended repeatedly, drawn through cannulas No. 1, No. 12 and No. 16 by means of a syringe and transferred to 50 ml Falcon tubes which are made up to 15 ml with complete medium. The contents of each Falcon tube are in each case filtered through a 70 μm Falcon filter insert and centrifuged at 1200 revolutions and room temperature for 10 minutes. The resulting pellet is in each case taken up in 250 μl of complete medium and the cell count is determined.

The number of cells in the suspension is adjusted to 3 times 10⁵ per ml and in each case 150 μl of this suspension are added to one well of the cell culture plates coated as described above. In the incubator, the plates are left to stand at 37° C., 5% by volume of CO₂ and 95% relative air humidity for two to three days.

Subsequently, the cells are laden with 2 μM Fluo-4 and 0.01% by volume of Pluronic F127 (Molecular Probes Europe BV, Leiden, the Netherlands) in HBSS buffer (Hank's buffered saline solution, Gibco Invitrogen GmbH, Karlsruhe, Germany) at 37° C. for 30 min, washed 3× with HBSS buffer and, after a further incubation of 15 minutes, used in the FLIPR assay at room temperature for Ca²⁺ measurement. The Ca²⁺-dependent fluorescence is measured before and after addition of substances (λₑₓ=488 nm, λₑₘ=540 nm). The quantification is effected by the measurement of the highest fluorescence intensity (FC, fluorescence counts) over the time.

### FLIPR Assay:

The FLIPR protocol consists of 2 substance additions. First, the compounds to be tested (10 μM) are pipetted onto the cells and the Ca²⁺ current is compared with the control (capsaicin 10 μM). This gives rise to the result in % activation based on the Ca²⁺ signal after addition of 10 μM capsaicin (CP). After incubation for 5 minutes, 100 nM capsaicin is applied and the current of Ca²⁺ is likewise determined.

Desensitizing agonists and antagonists lead to a suppression in the Ca²⁺ current. % inhibition is calculated compared to the maximum achievable inhibition with 10 μM capsaicin.

Triple determinations (n=3) are performed and they are repeated in at least 3 independent experiments (N=4).

### II. Functional Studies on the Vanilloid Receptor (VR1)

The agonistic or antagonistic action of the substances to be tested on vanilloid receptor (VR1) can also be determined with the assay which follows. In this assay, the Ca²⁺ current through the channel is quantified with the aid of a Ca²⁺-sensitive dye (Fluo-4 type, Molecular Probes, Europe BV, Leiden, the Netherlands) in a fluorescent imaging plate reader (FLIPR, Molecular Devices, Sunnyvale, USA).

### Method:

Chinese hamster ovary cells (CHO K1 cells, European Collection of Cell Cultures (ECACC) Great Britain) are transfected stably with the VR1 gene. For functional studies, these cells are plated out onto poly-D-lysine-coated black 96-well plates with a clear bottom (BD Biosciences, Heidelberg, Germany) in a density of 25 000 cells/well. The cells are incubated overnight at 37° C. and 5% CO₂ in a culture medium (Ham's Nutrient Mixture F12, 10% by volume of FCS (fetal calf serum), 18 μg/ml of L-proline). The next day, the cells are incubated with Fluo-4 (Fluo-4 2 μM, Pluronic F127 0.01% by volume, Molecular Probes in HBSS (Hank's buffered saline solution), Gibco Invitrogen GmbH, Karlsruhe, Germany) at 37° C. for 30 minutes. Subsequently, the plates are washed 3 times with HBSS buffer and, after a further incubation of 15 minutes at room temperature, used for Ca²⁺ measurement in FLIPR. The Ca²⁺-dependent fluorescence is measured before and after addition of the substances to be studied (wavelength λₑₓ=488 nm, λₑₘ=540 nm). The quantification is effected by measuring the highest fluorescence intensity (FC, fluorescence counts) over the time.

### FLIPR Assay:

The FLIPR protocol consists of 2 substance additions. First, the substances to be tested (10 μM) are pipetted onto the cells and the Ca current is compared with the control (capsaicin 10 μM) (% activation based on the Ca²⁺ signal after addition of 10 μM capsaicin). After incubation for 5 minutes, 100 nM capsaicin is applied and the current of Ca²⁺ is likewise determined.

### III. Formalin Test on Mice

The test to determine the antinociceptive action of the inventive 2,5-disubstituted thiazol-4-one derivatives is carried out in the formalin test on male mice (NMRI, body weight from 20 to 30 g, Iffa, Credo, Belgium).

In the formalin test, according to D. Dubuisson et al., Pain 1977, 4, 161-174, a distinction is drawn between the first (early) phase (from 0 to 15 minutes after the formalin injection) and the second (late) phase (from 15 to 60 minutes after the formalin injection). The early phase, as a direct reaction to the formalin injection, constitutes a model for acute pain, while the late phase is considered to be a model for persistent (chronic) pain (T. J. Coderre et al., Pain 1993, 52, 259-285). The corresponding literature descriptions are hereby included as a reference and form part of the disclosure.

The inventive 2,5-disubstituted thiazol-4-one derivatives are tested in the second phase of the formalin test in order to obtain statements regarding substance effects on chronic/inflammatory pain.

According to the administration type of the inventive compounds, the administration time of the inventive 2,5-disubstituted thiazol-4-one derivatives before the formalin injection is selected. The intravenous administration of 10 mg/kg of body weight of the test substances is effected 5 minutes before the formalin injection. This is done by a single subcutaneous formalin injection (20 μl, 1% aqueous solution) into the dorsal side of the right hind paw, such that a nociceptive reaction is induced in freely mobile test animals, which is manifested in obvious licking and biting of the paw affected.

Subsequently, for a test period of three minutes in the second (late) phase of the formalin test (from 21 to 24 minutes after the formalin injection), the nociceptive behavior is registered continuously by observing the animals. The pain behavior is quantified by summation of the seconds in which the animals exhibit licking and biting of the paw affected within the test period.

The comparison is in each case with control animals which, instead of the inventive compounds, receive vehicle (0.9% aqueous sodium chloride solution) before formalin administration. Based on the quantification of the pain behavior, the substance action in the formalin test is determined as the change relative to the corresponding control in percent.

After injection of substances which have antinociceptive activity in the formalin test, the behavior of the animals described, i.e. licking and biting, is reduced or eliminated.

### IV. Test for Analgesic Activity in the Writhing Test

The test of the inventive compounds of the general formula I for analgesic activity was carried out in phenylquinone-induced writhing in mice, modified according to I. C. Hendershot and J. Forsaith (1959), J. Pharmacol. Exp. Ther. 125, 237-240. The corresponding literature description is hereby included as a reference and forms part of the disclosure.

To this end, male NMRI mice with a weight of from 25 to 30 g were used. Groups of 10 animals per compound dose received, 10 minutes after intravenous administration of the compounds to be tested, 0.3 ml/mouse of a 0.02% aqueous solution of phenylquinone (phenylbenzoquinone, from Sigma, Deisenhofen, Germany; preparation of the solution with addition of 5% by weight of ethanol and storage in a water bath at 45° C.) which was applied intraperitoneally. The animals were placed individually into observation cages. With the aid of a push-button counter, the number of pain-induced stretching motions (so-called writhing reactions=arching of the back with stretching out of the rear extremities) was counted for from 5 to 20 minutes after the phenylquinone administration. The control employed was animals which had received only physiological saline. All compounds were tested in the standard dosage of 10 mg/kg.

The invention will be illustrated hereinafter with reference to examples. These illustrations are merely by way of example and do not restrict the general concept of the invention.

### EXAMPLES

The yields of the compounds prepared are not optimized.

All temperatures are uncorrected.

### Abbreviations

aq. aqueous
DCM dichloromethane
DMF dimethylformamide
EtOAc ethyl acetate
EtOH ethanol
sat. saturated
h hour
HOBT 1-hydroxybenzotriazole
MeOH methanol
RT room temperature
TBTU O-(benzotriazol-1-yl)-N,N,N′,N′-tetramethyluronium tetrafluoroborate

The chemicals and solvents used were purchased commercially from the conventional suppliers (Acros, Avocado, Aldrich, Bachem, Fluka, Lancaster, Maybridge, Merck, Sigma, TCI, etc.) or synthesized by methods known to those skilled in the art.

The stationary phase used for the column chromatography was silica gel 60 (0.040-0.063 mm) from E. Merck, Darmstadt.

The thin layer chromatography analyses were carried out with ready-to-use HPTLC plates, silica gel 60 F 254, from E. Merck, Darmstadt.

The mixing ratios of solvents, eluents or for chromatographic analyses are always reported in volume/volume.

The analysis was effected by mass spectroscopy and NMR.

### Synthesis of 2-phenyl-substituted thiazol-4-one derivatives

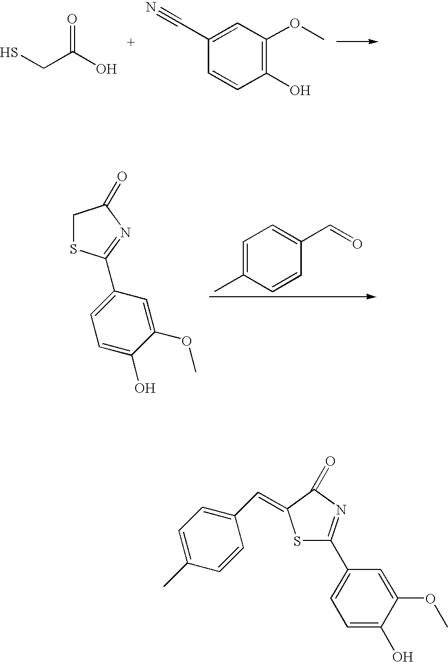

### 1a. Synthesis of 2-(4-hydroxy-3-methoxyphenyl)thiazol-4-one

4-Hydroxy-3-methoxybenzonitrile (25 g, 167.7 mmol) was dissolved in EtOH (355 ml) and admixed with thioglycolic acid (18.8 g, 167.7 mmol) and triethylamine (12.2 g, 120.4 mmol). The solvent was reduced down to 150 ml under reduced pressure and the precipitated solid was filtered off with suction, washed with a little EtOH and diisopropyl ether and dried.

The mother liquor was reduced further down to 75 ml and cooled.

The further solid which had precipitated out was filtered off with suction, washed with a little EtOH and diisopropyl ether and dried.

The combined precipitates were heated to reflux again in EtOH (50 ml). The precipitated solid was filtered off with suction, washed with a little EtOH and diisopropyl ether and dried. The desired product was obtained in a yield of 37% of theory (13.9 g).

### 1b. Synthesis of example compound 262: 2-(4-hydroxy-3-methoxyphenyl)-5-(4-methylbenzylidene)thiazol-4-one

2-(4-Hydroxy-3-methoxyphenyl)thiazol-4-one (1.34 mmol, 300 mg) and 4-methyl-benzaldehyde (194 mg, 1.61 mmol) were dissolved in acetic acid (7 g, 117.5 mmol) and admixed with sodium acetate (408 mg, 4.972 mmol). The reaction mixture was heated to reflux overnight and admixed with water, and the resulting precipitate was washed with butanol and diisopropyl ether and dried. The desired product was obtained as a solid (323 mg, 74% of theory).

### 2. Synthesis of 2-morpholinyl-substituted thiazol-4-one derivatives

### 2a. Synthesis of 2-morpholin-4-ylthiazol-4-one

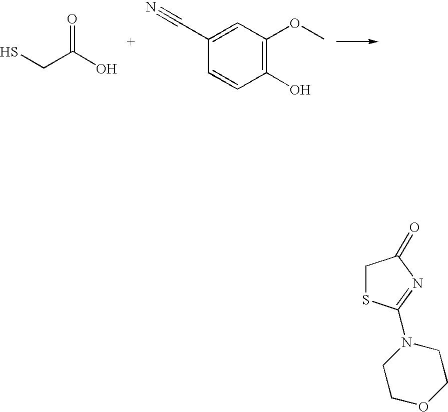

Cyanomorpholine (15.3 g, 136.3 mmol) and thioglycolic acid (12.5 g, 136.3 mmol) were introduced into a reaction vessel with pyridine (12.2 ml). The reaction vessel was heated to 10° C. in a Pyrex tube. After one hour, the reaction was ended and the mixture was cooled.

The resulting precipitate was filtered off with suction, washed with a little EtOH and diisopropyl ether and dried. The desired product was obtained as a white solid (21.5 g, 85% of theory).

### 2b. Synthesis of example compound 58: 5-decylidene-2-morpholin-4-ylthiazol-4-one

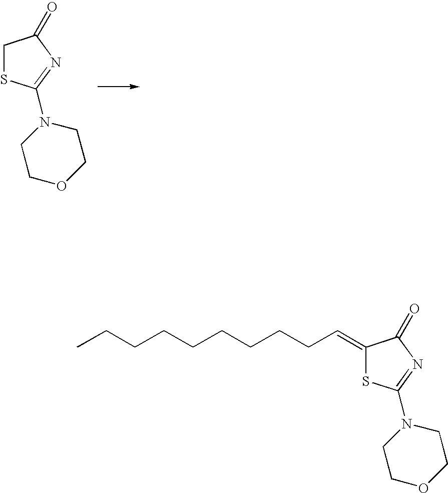

2-Morpholin-4-ylthiazol-4-one (500 mg, 2.7 mmol) and n-decanal (503 mg, 3.22 mol) were dissolved in EtOH (10 ml) and admixed with triethylamine (815 mg). The reaction mixture was heated to reflux for 48 h. The precipitated solid was filtered off with suction and dried. For further purification, the precipitate was heated in a little butanol (3 ml). The further solid which had precipitated out was filtered off with suction and dried. The desired product was obtained as a solid (82 mg, 10% of theory).

### 2c. Synthesis of example compound 17: 2-morpholin-4-yl-5-(4-phenoxybenzylidene)thiazol-4-one

2-Morpholin-4-yl-thiazol-4-one (1 g, 5.4 mmol) and 3-phenoxybenzaldehyde (1.27 g, 6.43 mmol) were dissolved in acetic acid (28 g) and admixed with sodium acetate (1.63 g, 19.9 mmol). The reaction mixture was heated to reflux overnight and stirred at RT for 24 h, and the resulting precipitate was washed with butanol and diisopropyl ether and dried. The desired product was obtained as a solid (1.37 g, 70% of theory).

### 2d. Synthesis of example compound 19: 2-morpholin-4-yl-5-(3-p-tolylallylidene)thiazol-4-one

2-Morpholin-4-ylthiazol-4-one (530 mg, 2.85 mmol) and 3-p-tolylpropenal (500 mg, 3.42 mmol) were dissolved in acetic acid (15 g) and admixed with sodium acetate (865 mg, 10.5 mmol). The reaction mixture was heated to reflux for 5 h and stirred at RT for 48 h. The solvent was removed under reduced pressure and the residue was taken up in water and EtOAc. The organic phase was dried over magnesium sulfate, the solvent was removed under reduced pressure and the residue was dried. The residue was heated in butanol (20 ml) and the precipitated solid was washed with a little diisopropyl ether and dried. The desired product was obtained as a solid (224 mg, 21% of theory).

### 2e. Synthesis of example compound 50: 5-(2,3-diphenylallylidene)-2-morpholin-4-ylthiazol-4-one

2-Morpholin-4-ylthiazol-4-one (500 mg, 2.7 mmol) and diphenylacrolein (670 mg, 3.22 mol) were dissolved in EtOH (10 ml) and admixed with triethylamine (815 mg). The reaction mixture was heated to reflux for 48 h. The precipitated solid was filtered off with suction and dried. The desired product was obtained as a solid (862 mg, 85% of theory).

### 3. Synthesis of 2-thiomorpholinyl-substituted thiazol-4-one derivatives

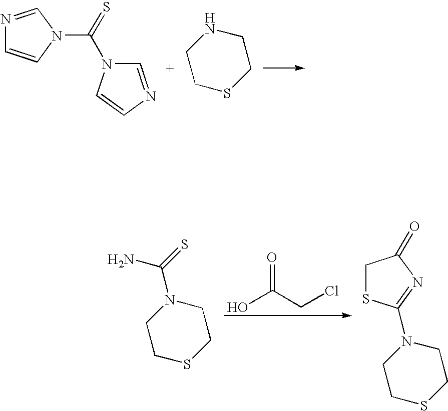

### 3a. Synthesis of thiomorpholine-4-thiocarboxamide

1,1′-Thiocarbonyldiimidazole (45.4 g, 254 mmol) was suspended in THF (605 g). Thiomorpholine (25 g, 242.6 mmol) was dissolved in a little THF and added to the suspension. The reaction mixture was stirred at RT for 1 h. 7 N ammonia solution in MeOH (346 g) was added thereto and the reaction mixture was stirred at 40° C. overnight. The solvent was removed under reduced pressure, and the residue was washed with a little cold butanol and ether and dried. The desired product was obtained in an amount of 31.5 g.

### 3b. Synthesis of 2-thiomorpholin-4-ylthiazol-4-one

Thiomorpholine-4-thiocarboxamide (38 g, 234 mmol) was suspended with chloroacetic acid (23 g, 243 mmol) in pyridine (87 g). The suspension was heated in a microwave vessel for 45 min and left to stand overnight. The precipitated solid was filtered off with suction, washed with a little butanol and ether and dried. The desired product was obtained as a solid (21.7 g, 46% of theory).

### 3c. Synthesis of example compound 85: 5-(2-chloro-3-phenylallylidene)-2-thiomorpholin-4-ylthiazol-4-one

2-Thiomorpholin-4-ylthiazol-4-one (800 mg, 3.96 mmol) and alpha-chloro-cinnamaldehyde (789 mg, 4.752 mmol) were dissolved in acetic acid (21 g) and admixed with sodium acetate (1.2 g, 14.6 mmol). The reaction mixture was heated to reflux for 4 h and added to water (100 ml). The aqueous phase was extracted with DCM and the solvent was removed under reduced pressure. The aqueous phase was left to stand overnight. A second phase formed, which was concentrated under reduced pressure. The residue was heated in butanol (1 ml) and the precipitated solid was washed with a little diisopropyl ether and dried. The desired product was obtained as a solid (138 mg, 10% of theory).

### 3d. Synthesis of example compound 86: 3-(4-oxo-2-thiomorpholin-4-yl-4H-thiazol-5-ylidenemethyl)benzoic acid

2-Thiomorpholin-4-ylthiazol-4-one (5 g, 25 mmol) and 3-carboxybenzaldehyde (4.45 g, 29.7 mmol) were dissolved in acetic acid (130 g) and admixed with sodium acetate (7.5 g, 91.5 mmol). The reaction mixture was heated to reflux overnight. After cooling, a precipitate formed, which was filtered off with suction, washed with water, butanol and diisopropyl ether and dried. The desired product was obtained as a solid (4.91 g, 60% of theory).

### 3e. Synthesis of 1-(6-chloropyridin-2-yl)piperazine

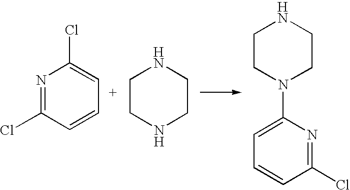

piperazine (22.4 g, 260.2 mmol) was dissolved in DMSO (462 g) and admixed with 2,6-dichloropyridine (35 g, 236 mmol). The reaction mixture was stirred at 110° C. overnight. The reaction mixture was admixed with portions of sat. aq. sodium hydrogencarbonate solution (250 ml in total), and the resulting precipitate was filtered off with suction. The filtrate was extracted repeatedly with EtOAc and the combined organic phases were dried, and the solvent was removed under reduced pressure. The residue was distilled at 2.5-1 mbar. The product was obtained at top temperature 120° C. and bottom temperature 165° C. Yield: 17.7 g (38% of theory).

### 3f. Synthesis of example compound 108: 5-{3-[4-(6-chloropyridin-2-yl)piperazine-1-carbonyl]benzylidene}-2-thiomorpholin-4-ylthiazol-4-one

3-(4-Oxo-2-thiomorpholin-4-yl-4H-thiazol-5-ylidenemethyl)benzoic acid (500 mg, 1.5 mmol), 1-(6-chloropyridin-2-yl)piperazine (295 mg, 1.5 mmol), TBTU (480 mg, 1.5 mmol), HOBT (201 mg, 1.5 mmol) and diisopropylethylamine (643 mg, 4.5 mmol) were dissolved in THF (10 ml) and stirred at RT overnight. The resulting precipitate was filtered off with suction, washed with EtOAc and ether, and dried.

Yield: 579 mg (75% of theory)

### 3g. Synthesis of example compound 112: 3-(4-oxo-2-thiomorpholin-4-yl-4H-thiazol-5-ylidenemethyl)benzoic acid N′-(2-chloro-4-trifluoromethylphenyl)hydrazide

3-(4-Oxo-2-thiomorpholin-4-yl-4H-thiazol-5-ylidenemethyl)benzoic acid (500 mg, 1.5 mmol), 1-(2-chloro-4-(trifluoromethyl)phenyl)hydrazine (315 mg, 1.5 mmol), TBTU (480 mg, 1.5 mmol), HOBT (201 mg, 1.5 mmol) and diisopropylethylamine (643 mg, 4.5 mmol) were dissolved in THF (10 ml) and stirred at RT for 48 h. The resulting precipitate was filtered off with suction, washed with EtOAc and ether, and dried.

Yield: 305 mg (39% of theory).

### 4. Synthesis of 2-piperazinyl-substituted thiazol-4-one derivatives

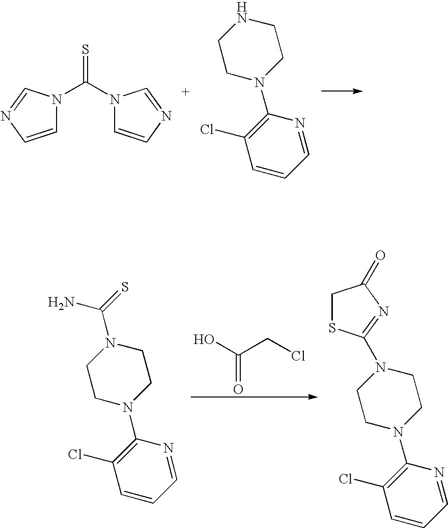

### 4a. Synthesis of 1-(3-chloropyridin-2-yl)piperazine

Piperazine (15 g, 174 mmol) was dissolved in DMSO (309 g) and admixed with 2,3-dichloropyridine (23.4 g, 158 mmol). The reaction mixture was stirred at 110° C. for 60 h. The reaction mixture was admixed with portions of sat. aq. sodium hydrogencarbonate solution (250 ml in total), and the resulting precipitate was filtered off with suction. The filtrate was extracted repeatedly with EtOAc (150 ml each time) and the combined organic phases were dried, and the solvent was removed under reduced pressure. The residue was distilled at 2.1-1 mbar. The product was obtained at top temperature 95° C. and bottom temperature 155° C. Yield: 9.6 g (28% of theory).

### 4b. Synthesis of 4-(3-chloropyridin-2-yl)piperazine-1-thiocarboxamide

1,1′-Thiocarbonyldiimidazole (3.8 g, 21.2 mmol) was suspended in THF (23.4 g). 1-(3-Chloropyridin-2-yl)piperazine (4 g, 20.2 mmol) was dissolved in a little THF and added to the suspension. The reaction mixture was stirred at RT for 1 h. 7 N ammonia solution in MeOH (28.9 g) was added thereto and the reaction mixture was stirred at 40° C. overnight. The solvent was removed under reduced pressure, and the residue was washed with a little cold butanol and ether and dried. The desired product was obtained in an amount of 4.1 g (78% of theory).

### 4c. Synthesis of 2-[4-(3-chloropyridin-2-yl)piperazin-1-yl]thiazol-4-one

4-(3-Chloropyridin-2-yl)piperazine-1-thiocarboxamide (8.1 g, 31.9 mmol) was suspended with chloroacetic acid (3.1 g, 33.1 mmol) in EtOH (7 g). The suspension was heated to 85° C. in a microwave vessel for 60 min and left to stand overnight. The precipitated solid was filtered off with suction, washed with a little butanol and ether and dried. The precipitate was heated in a little butanol (10 ml) and the remaining solid was filtered off with suction. The desired product was obtained as a solid (2.34 g, 25% of theory).

### 4d. Synthesis of example compound 95: 2-[4-(3-chloropyridin-2-yl)piperazin-1-yl]-5-(4-hydroxy-3-methoxybenzylidene)-thiazol-4-one

2-[4-(3-Chloropyridin-2-yl)piperazin-1-yl]thiazol-4-one (500 mg, 1.7 mmol) and vanillin (308 mg, 2.02 mmol) were dissolved in acetic acid (9 g) and admixed with sodium acetate (514 mg, 6.3 mmol). The reaction mixture was heated to reflux overnight. After cooling, a precipitate formed, which was filtered off with suction, washed with diisopropyl ether and dried. The precipitate was heated in a little butanol (8 ml) and hot-filtered. The precipitate was discarded and the filtrate was left to stand. The resulting precipitate was filtered off with suction and dried. The desired product was obtained as a solid (157 mg, 22% of theory).

### 4e. Synthesis of example compound 49: 2-[4-(3-chloropyridin-2-yl)piperazin-1-yl]-5-naphthalen-2-ylmethylenethiazol-4-one

2-[4-(3-Chloropyridin-2-yl)piperazin-1-yl]thiazol-4-one (500 mg, 1.7 mmol) and naphthalene-2-carbaldehyde (263 mg, 1.7 mmol) were dissolved in EtOH (10 ml) and admixed with triethylamine (512 mg, 5.1 mmol). The reaction mixture was heated to reflux for 72 h. After cooling, a precipitate formed, which was filtered off with suction, washed with butanol and diisopropyl ether and dried. The desired product was obtained as a solid (295 mg, 40% of theory).

### 4f. Synthesis of example compound 90: 2-[4-(3-chloropyridin-2-yl)piperazin-1-yl]-5-quinolin-3-ylmethylenethiazol-4-one

2-[4-(3-Chloropyridin-2-yl)piperazin-1-yl]thiazol-4-one (500 mg, 1.7 mmol) and 3-quinolinecarboxaldehyde (318 mg, 2.02 mmol) were dissolved in acetic acid (9 g) and admixed with sodium acetate (514 mg, 6.3 mmol). The reaction mixture was heated to reflux overnight. After cooling, the mixture was admixed with water (100 ml). The precipitate formed was filtered off with suction and heated in a little butanol (4 ml). The precipitate formed was filtered off with suction, washed with a little butanol and ether and dried. The desired product was obtained as a solid (367 mg, 50% of theory).

### 5. Synthesis of 2-(4-benzylpiperazinyl)-substituted thiazol-4-one derivatives

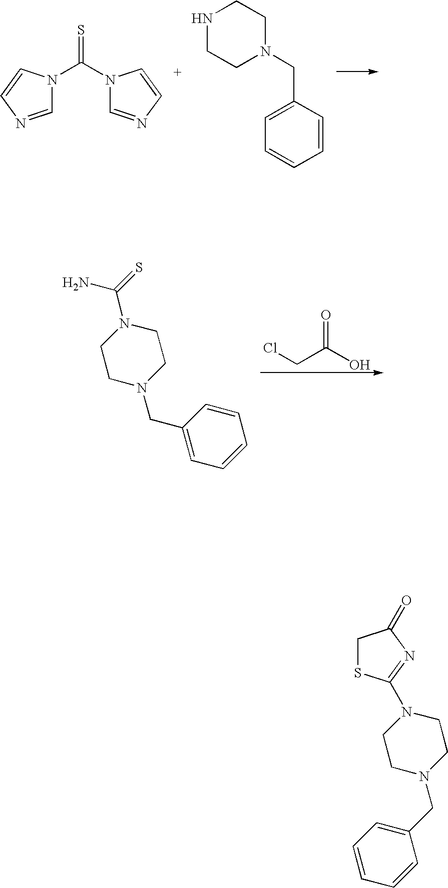

### 5a. Synthesis of 4-benzylpiperazine-1-thiocarboxamide

1,1′-Thiocarbonyldiimidazole (18 g, 101.2 mmol) was suspended in THF (112 g). 1-Benzylpiperazine (17 g, 96.5 mmol) was dissolved in a little THF and added to the suspension. The reaction mixture was stirred at RT for 1 h. 7 N ammonia solution in MeOH (137 g) was added thereto and the reaction mixture was stirred at 40° C. overnight. The solvent was removed under reduced pressure, and the residue was heated in a little butanol (25 ml). After cooling, a precipitate was obtained, which was washed with a little cold butanol and ether and then dried. The desired product was obtained in an amount of 14.1 g.

### 5b. Synthesis of 2-(4-benzylpiperazin-1-yl)thiazol-4-one

4-Benzylpiperazine-1-thiocarboxamide (14 g, 59.5 mmol) was suspended with chloroacetic acid (5.8 g, 61.9 mmol) in pyridine (55 g). The suspension was heated to 120° C. in a microwave vessel for 45 min. The reaction mixture was concentrated fully under reduced pressure and the residue was heated in MeOH (150 ml). The precipitated solid was filtered off with suction, washed with ether and dried. The desired product was obtained as a solid (8.9 g, 55% of theory).

### 5c. Synthesis of example compound 104: 2-(4-benzylpiperazin-1-yl)-5-(4-tert-butylbenzylidene)thiazol-4-one

2-(4-Benzylpiperazin-1-yl)thiazol-4-one (500 mg, 1.8 mmol) and 4-tert-butyl-benzaldehyde (353 mg, 2.18 mmol) were dissolved in acetic acid (9.5 g) and admixed with sodium acetate (552 mg, 6.7 mmol). The reaction mixture was heated to reflux overnight. After cooling, the mixture was admixed with water (50 ml). The precipitate formed was filtered off with suction and heated in a little butanol (1 ml). The resulting precipitate was filtered off with suction, washed with a little butanol and ether and dried. The desired product was obtained as a solid (245 mg, 32% of theory).

### 5d. Synthesis of example compound 158: 2-(4-benzylpiperazin-1-yl)-5-(4-pentafluorosulfanylbenzylidene)thiazol-4-one

2-(4-Benzylpiperazin-1-yl)thiazol-4-one (350 mg, 1.3 mmol) and 4-(pentafluorosulfanyl)benzaldehyde (353 mg, 1.52 mmol) were dissolved in acetic acid (6.7 g) and admixed with sodium acetate (387 mg, 4.7 mmol). The reaction mixture was heated to reflux overnight. The resulting precipitate was filtered off with suction, washed with a little butanol and ether and dried. The desired product was obtained as a solid (108 mg).

### 6. Synthesis of 2-aminophenyl-substituted thiazol-4-one derivatives

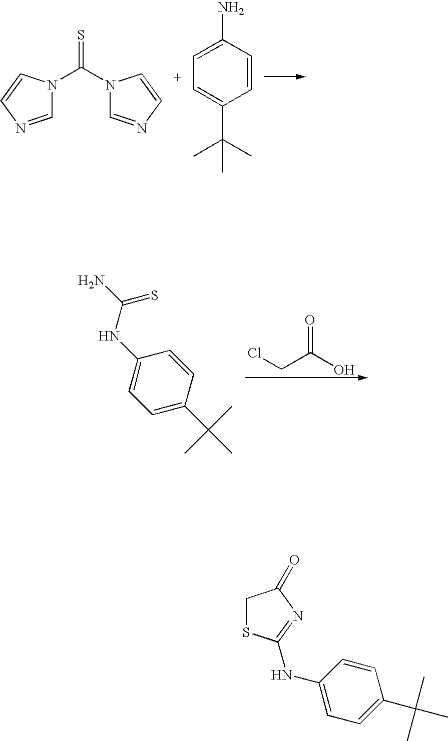

### 6a. Synthesis of (4-tert-butylphenyl)thiourea

1,1′-Thiocarbonyldiimidazole (12.5 g, 70.4 mmol) was suspended in THF (167 g). 4-tert-Butylaniline (10 g, 67 mmol) was dissolved in a little THF and added to the suspension. The reaction mixture was stirred at RT for 1 h. 7 N ammonia solution in MeOH (95 g) was added thereto and the reaction mixture was stirred at 40° C. for 48 h. The solvent was removed under reduced pressure and the residue was heated in a little butanol (25 ml). After cooling, a precipitate was obtained, which was washed with a little cold butanol and ether and then dried. The desired product was obtained in an amount of 14 g.

### 6b. Synthesis of 2-(4-tert-butylphenylamino)thiazol-4-one

(4-tert-Butylphenyl)thiourea (14 g, 68.7 mmol) was suspended with chloroacetic acid (6.7 g, 71.49 mmol) in EtOH (15 g). The suspension was heated to 85° C. in a microwave vessel for 60 min. The precipitated solid was filtered off with suction, washed with a little butanol and diisopropyl ether and dried. The desired product was obtained as a solid (4.9 g).

### 6c. Synthesis of example compound 263: 2-(4-tert-butylphenylamino)-5-(4-methylbenzylidene)thiazol-4-one

2-(4-tert-Butylphenylamino)thiazol-4-one (550 mg, 2.2 mmol) and 4-methylbenzaldehyde (319 mg, 1.02 mmol) were dissolved in acetic acid (11.6 g) and admixed with sodium acetate (673 mg, 8.2 mmol). The reaction mixture was heated to reflux for 5 h and stirred at RT for 72 h. The resulting precipitate was filtered off with suction, washed with a little water and hexane and dried. The desired product was obtained as a solid (525 mg, 67% of theory).

### 7. Synthesis of 2-aminobenzyl-substituted thiazol-4-one derivatives

### 7a. Synthesis of benzo[1.3]dioxol-5-ylmethylthiourea

1,1′-Thiocarbonyldiimidazole (12.3 g, 69.5 mmol) was suspended in THF (167 g). 3,4-Methylenedioxybenzylamine (10 g, 66.2 mmol) was dissolved in a little THF and added to the suspension. The reaction mixture was stirred at RT for 1 h. 7 N ammonia solution in MeOH (94.5 g) was added thereto and the reaction mixture was stirred at 40° C. for 24 h. The solvent was removed under reduced pressure and the residue was heated in a little butanol (15 ml). After cooling, a precipitate was obtained, which was washed with a little cold butanol and ether and then dried. The desired product was obtained in an amount of 11.1 g (80% of theory).

### 7b. Synthesis of 2-[(benzo[1,3]dioxol-5-ylmethyl)amino]thiazol-4-one

Benzo[1.3]dioxol-5-ylmethylthiourea (11 g, 52.4 mmol) was suspended with chloroacetic acid (5.1 g, 54.4 mol) in pyridine (20 g). The suspension was heated to 110° C. in a microwave vessel for 60 min. The reaction mixture was concentrated under reduced pressure and the residue was dissolved with a little butanol (15 ml) while heating. After cooling, a precipitate formed, which was filtered off with suction, washed with a little butanol and diisopropyl ether and dried. The desired product was obtained as a solid (4.0 g, 30% of theory).

### 7c. Synthesis of example compound 243: 2-[(benzo[1,3]dioxol-5-ylmethyl)amino]-5-(4-trifluoromethoxybenzylidene)-thiazol-4-one

2-[(Benzo[1,3]dioxol-5-ylmethyl)amino]thiazol-4-one (350 mg, 1.4 mmol) and 4-(trifluoromethoxy)benzaldehyde (319 mg, 1.68 mmol) were dissolved in acetic acid (7.3 g) and admixed with sodium acetate (425 mg, 5.2 mmol). The reaction mixture was heated to reflux for 14 h. The reaction mixture was admixed with water (30 ml), and the resulting precipitate was filtered off with suction, washed with a little water and hexane and dried. The desired product was obtained as a solid (460 mg, 78% of theory).

### 8. Synthesis of 2-urea-substituted thiazol-4-one derivatives

### 8a. Synthesis of 2-aminothiazol-4-one

Thiourea (30 g, 395 mmol) was suspended with chloroacetic acid (38.7 g, 410 mol) in pyridine (146 g). The suspension was heated in a microwave vessel for 45 min. The reaction mixture was concentrated under reduced pressure and the residue was dissolved in EtOH (250 ml) while heating. After cooling, a precipitate formed, which was filtered off with suction, washed with ether and dried. The desired product was obtained as a solid (26.9 g).

### 8b. Synthesis of 2-amino-5-(4-trifluoromethylbenzylidene)-4-thiazol-4-one

2-Aminothiazol-4-one (6 g, 51.7 mmol) and 4-(trifluoromethyl)benzaldehyde (10.8 g, 62 mmol) were dissolved in acetic acid (271 g) and admixed with sodium acetate (15.7 g, 191 mmol). The reaction mixture was heated to reflux for 5 h. The reaction mixture was admixed with water (30 ml), and the resulting precipitate was filtered off with suction, washed with EtOH and ether and dried. The desired product was obtained as a solid (8.8 g, 63% of theory).

### 8c. Synthesis of example compound 106: 1-[4-oxo-5-(4-trifluoromethylbenzylidene)-4,5-dihydrothiazol-2-yl]-3-(4-trifluoromethylphenyl)urea

2-Amino-5-(4-trifluoromethylbenzylidene)-4-thiazol-4-one (436 mg, 1.6 mmol) was dissolved in DMSO (28 g) and admixed with triethylamine (179 mg, 1.76 mmol) and 1-isocyanato-4-trifluoromethylbenzene (436 mg, 1.6 mmol). The reaction mixture was stirred at RT overnight and added to ice-water (50 ml). The resulting precipitate was filtered off with suction and heated to reflux in a little acetone. A little water was added thereto and the reaction mixture was left to stand. The resulting precipitate was filtered off with suction, washed with water and dried. The desired product was obtained as a white solid (553 mg, 75% of theory).

The following inventive 2,5-disubstituted thiazol-4-one derivatives were prepared as described under 1. to 8.

| | |
|---|---|
| 1 | 5-[3-(4-methoxyphenyl)but-2-enylidene]-2-morpholin-4-ylthiazol-4-one |
| 2 | 2-morpholin-4-yl-5-phenethylidenethiazol-4-one hydrochloride |
| 3 | 2-morpholin-4-yl-5-(2-o-tolylethylidene)thiazol-4-one hydrochloride |
| 4 | 2-morpholin-4-yl-5-(3-phenylallylidene)thiazol-4-one |
| 5 | 2-morpholin-4-yl-5-(3-phenylbutylidene)thiazol-4-one |
| 6 | 2-morpholin-4-yl-5-(3-phenylpropylidene)thiazol-4-one |
| 7 | 5-[3-(4-fluorophenyl)allylidene]-2-morpholin-4-ylthiazol-4-one |
| 8 | N-[4-(2-morpholin-4-yl-4-oxo-4H-thiazol-5-ylidenemethyl)phenyl]acetamide |
| 9 | 5-benzo[1,3]dioxol-5-ylmethylene-2-morpholin-4-ylthiazol-4-one |
| 10 | 2-morpholin-4-yl-5-(3-m-tolylallylidene)thiazol-4-one |
| 11 | 5-(3-iodo-4,5-dimethoxybenzylidene)-2-morpholin-4-ylthiazol-4-one |
| 12 | 2-morpholin-4-yl-5-(3-phenylsulfanylallylidene)thiazol-4-one |
| 13 | 5-(3-benzyloxy-4-methoxybenzylidene)-2-morpholin-4-ylthiazol-4-one |
| 14 | 5-(4-methylbenzylidene)-2-thiomorpholin-4-ylthiazol-4-one hydrobromide |
| 15 | N-[4-(2-morpholin-4-yl-4-oxo-4H-thiazol-5-ylidenemethyl)phenyl]amine |
| 16 | 5-(4-butoxybenzylidene)-2-morpholin-4-ylthiazol-4-one |
| 17 | 2-morpholin-4-yl-5-(4-phenoxybenzylidene)thiazol-4-one |
| 18 | 2-morpholin-4-yl-5-(3-phenoxybenzylidene)thiazol-4-one |
| 19 | 2-morpholin-4-yl-5-(3-p-tolylallylidene)thiazol-4-one |
| 20 | 5-(3-benzyloxybenzylidene)-2-morpholin-4-ylthiazol-4-one |
| 21 | 5-(4-benzyloxybenzylidene)-2-morpholin-4-ylthiazol-4-one |
| 22 | 2-thiomorpholin-4-yl-5-(2-trifluoromethylbenzylidene)thiazol-4-one |
| 23 | 5-(4-bromobenzylidene)-2-thiomorpholin-4-ylthiazol-4-one |
| 24 | 2-thiomorpholin-4-yl-5-(4-trifluoromethoxybenzylidene)thiazol-4-one |
| 25 | 5-(3-phenylallylidene)-2-thiomorpholin-4-ylthiazol-4-one |
| 26 | 2-morpholin-4-yl-5-octylidenethiazol-4-one |
| 27 | 5-(4-benzyloxy-3-methoxybenzylidene)-2-morpholin-4-ylthiazol-4-one |
| 28 | 5-(3,4-bis(benzyloxy)benzylidene)-2-morpholin-4-ylthiazol-4-one |
| 29 | 5-butylidene-2-morpholin-4-ylthiazol-4-one |
| 30 | 2-[4-(3-chloropyridin-2-yl)piperazin-1-yl]-5-(4-methylbenzylidene)thiazol- |
| | 4-one |
| 31 | 2-[4-(3-chloropyridin-2-yl)piperazin-1-yl]-5-(3-phenylallylidene)thiazol-4-one |
| 32 | 5-hexylidene-2-morpholin-4-ylthiazol-4-one |
| 33 | 2-morpholin-4-yl-5-(3-p-tolylbut-2-enylidene)thiazol-4-one |
| 34 | 5-[3-(4-tert-butylphenyl)but-2-enylidene]-2-morpholin-4-ylthiazol-4-one |
| 35 | 5-(4-methoxynaphthalen-1-ylmethylene)-2-morpholin-4-ylthiazol-4-one |
| 36 | 2-morpholin-4-yl-5-(4-trifluoromethoxybenzylidene)thiazol-4-one |
| 37 | 2-[4-(3-methoxyphenyl)piperazin-1-yl]-5-(3-phenylallylidene)thiazol-4-one |
| 38 | 5-[3-(4-tert-butylphenyl)allylidene]-2-[4-(3-methoxyphenyl)piperazin-1-yl]- |
| | thiazol-4-one |
| 39 | 5-(4-hydroxy-3-methoxybenzylidene)-2-thiomorpholin-4-ylthiazol-4-one |
| 40 | 5-[3-(4-hydroxy-3-methoxyphenyl)allylidene]-2-thiomorpholin-4-ylthiazol- |
| | 4-one |
| 41 | 5-naphthalen-1-ylmethylene-2-thiomorpholin-4-ylthiazol-4-one |
| 42 | 5-benzylidene-2-thiomorpholin-4-ylthiazol-4-one |
| 43 | 2-morpholin-4-yl-5-[3-(4-trifluoromethylphenyl)but-2-enylidene]thiazol-4-one |
| 44 | 2-morpholin-4-yl-5-[3-(3-trifluoromethylphenyl)but-2-enylidene]thiazol-4-one |
| 45 | 5-(3-methylbutylidene)-2-morpholin-4-ylthiazol-4-one |
| 46 | 2-[4-(3-chloropyridin-2-yl)piperazin-1-yl]-5-(2-trifluoromethylbenzylidene)- |
| | thiazol-4-one |
| 47 | 5-(4-bromobenzylidene)-2-[4-(3-chloropyridin-2-yl)piperazin-1-yl]thiazol- |
| | 4-one |
| 48 | 5-benzylidene-2-[4-(3-chloropyridin-2-yl)piperazin-1-yl]thiazol-4-one |
| 49 | 2-[4-(3-chloropyridin-2-yl)piperazin-1-yl]-5-naphthalen-2-ylmethylenethiazol- |
| | 4-one |
| 50 | 5-(2,3-diphenylallylidene)-2-morpholin-4-ylthiazol-4-one |
| 51 | 2-morpholin-4-yl-5-phenanthren-9-ylmethylenethiazol-4-one |
| 52 | 2-morpholin-4-yl-5-quinolin-3-ylmethylenethiazol-4-one |
| 53 | 2-(4-ethylpiperazin-1-yl)-5-(4-methylbenzylidene)thiazol-4-one hydrobromide |
| 54 | 5-naphthalen-2-ylmethylene-2-thiomorpholin-4-ylthiazol-4-one |
| 55 | 2-[4-(4-methoxyphenyl)piperazin-1-yl]-5-(3-phenylallylidene)thiazol-4-one |
| 56 | 2-[4-(2-chlorophenyl)piperazin-1-yl]-5-(3-phenylallylidene)thiazol-4-one |
| 57 | 5-(3-phenylallylidene)-2-(4-phenylpiperazin-1-yl)thiazol-4-one |
| 58 | 5-decylidene-2-morpholin-4-ylthiazol-4-one |
| 59 | 5-[3-(4-tert-butylphenyl)allylidene]-2-morpholin-4-ylthiazol-4-one |
| 60 | 2-(2-morpholin-4-yl-4-oxo-4H-thiazol-5-ylidenemethyl)benzoic acid |
| | triethylammonium salt |
| 61 | 5-(2-bromo-3-phenylallylidene)-2-[4-(3-chloropyridin-2-yl)piperazin-1-yl]- |
| | thiazol-4-one |
| 62 | 5-[3-(4-tert-butylphenyl)allylidene]-2-[4-(3-chloropyridin-2-yl)piperazin-1-yl]- |
| | thiazol-4-one |
| 63 | 2-(4-benzylpiperazin-1-yl)-5-(4-methylbenzylidene)thiazol-4-one |
| 64 | 2-(4-phenylpiperazin-1-yl)-5-quinolin-3-ylmethylenethiazol-4-one |
| 65 | 2-[4-(3-chloropyridin-2-yl)piperazin-1-yl]-5-(2-methyl-3-phenylallylidene)- |
| | thiazol-4-one |
| 66 | 2-[4-(3-chloropyridin-2-yl)piperazin-1-yl]-5-naphthalen-1-ylmethylenethiazol- |
| | 4-one |
| 67 | 5-(2-chloro-3-phenylallylidene)-2-[4-(3-chloropyridin-2-yl)piperazin-1-yl]- |
| | thiazol-4-one |
| 68 | 5-(4-hydroxy-3-methoxybenzylidene)-2-(4-phenylpiperazin-1-yl)thiazol-4-one |
| 69 | 5-phenanthren-9-ylmethylene-2-(4-phenylpiperazin-1-yl)thiazol-4-one |
| 70 | 5-(6-methoxynaphthalen-2-ylmethylene)-2-(4-phenylpiperazin-1-yl)thiazol- |
| | 4-one |
| 71 | 5-naphthalen-1-ylmethylene-2-(4-phenylpiperazin-1-yl)thiazol-4-one |
| 72 | 5-(4-bromobenzylidene)-2-(4-phenylpiperazin-1-yl)thiazol-4-one |
| 73 | 5-(4-methylbenzylidene)-2-(4-phenylpiperazin-1-yl)thiazol-4-one |
| 74 | 2-(4-phenylpiperazin-1-yl)-5-(2-trifluoromethylbenzylidene)thiazol-4-one |
| 75 | 3-(4-oxo-2-thiomorpholin-4-yl-4H-thiazol-5-ylidene)-1,3-dihydroindol-2-one |
| 76 | 3-(2-morpholin-4-yl-4-oxo-4H-thiazol-5-ylidene)-1,3-dihydroindol-2-one |
| 77 | 2-[4-(2-fluorophenyl)piperazin-1-yl]-5-(4-methylbenzylidene)thiazol-4-one |
| 78 | 5-(4-tert-butylbenzylidene)-2-[4-(2-fluorophenyl)piperazin-1-yl]thiazol-4-one |
| 79 | 2-[4-(2-fluorophenyl)piperazin-1-yl]-5-(3-phenylallylidene)thiazol-4-one |
| 80 | 5-[3-(4-tert-butylphenyl)allylidene]-2-[4-(2-fluorophenyl)piperazin-1-yl]- |
| | thiazol-4-one |
| 81 | 2-[4-(2-fluorophenyl)piperazin-1-yl]-5-(4-trifluoromethylbenzylidene)thiazol-4- |
| | one |
| 82 | 2-[4-(2-fluorophenyl)piperazin-1-yl]-5-(4-trifluoromethoxybenzylidene)- |
| | thiazol-4-one |
| 83 | 5-(2-chloro-3-phenylallylidene)-2-[4-(2-fluorophenyl)piperazin-1-yl]thiazol- |
| | 4-one |
| 84 | 2-[4-(2-fluorophenyl)piperazin-1-yl]-5-naphthalen-1-ylmethylenethiazol-4-one |
| 85 | 5-(2-chloro-3-phenylallylidene)-2-thiomorpholin-4-ylthiazol-4-one |
| 86 | 3-(4-oxo-2-thiomorpholin-4-yl-4H-thiazol-5-ylidenemethyl)benzoic acid |
| 87 | 4-(4-oxo-2-thiomorpholin-4-yl-4H-thiazol-5-ylidenemethyl)benzoic acid |
| 88 | 5-{3-[4-(3-chloropyridin-2-yl)piperazine-1-carbonyl]benzylidene}-2-thio- |
| | morpholin-4-ylthiazol-4-one |
| 89 | 5-{4-[4-(3-chloropyridin-2-yl)piperazine-1-carbonyl]benzylidene}-2-thio- |
| | morpholin-4-ylthiazol-4-one |
| 90 | 2-[4-(3-chloropyridin-2-yl)piperazin-1-yl]-5-quinolin-3-ylmethylenethiazol- |
| | 4-one |
| 91 | 2-[4-(3-chloropyridin-2-yl)piperazin-1-yl]-5-(4-trifluoromethylbenzylidene)- |
| | thiazol-4-one |
| 92 | 2-(2,3-dihydroindol-1-yl)-5-(4-methylbenzylidene)thiazol-4-one |
| 93 | 2-(2,3-dihydroindol-1-yl)-5-(4-trifluoromethylbenzylidene)thiazol-4-one |
| 94 | 5-[4-(morpholine-4-carbonyl)benzylidene]-2-thiomorpholin-4-ylthiazol-4-one |
| 95 | 2-[4-(3-chloropyridin-2-yl)piperazin-1-yl]-5-(4-hydroxy-3-methoxy- |
| | benzylidene)thiazol-4-one |
| 96 | 1-[4-oxo-5-(4-trifluoromethylbenzylidene)-4,5-dihydrothiazol-2-yl]-3-(2- |
| | trifluoromethylphenyl)urea |
| 97 | 2-(4-benzylpiperazin-1-yl)-5-quinolin-3-ylmethylenethiazol-4-one |
| 98 | 2-(4-benzylpiperazin-1-yl)-5-naphthalen-2-ylmethylenethiazol-4-one |
| 99 | 2-(4-benzylpiperazin-1-yl)-5-(3-phenoxybenzylidene)thiazol-4-one |
| 100 | 2-(4-benzylpiperazin-1-yl)-5-(4-trifluoromethylbenzylidene)thiazol-4-one |
| 101 | 1-[5-(4-methylbenzylidene)-4-oxo-4,5-dihydrothiazol-2-yl]-3-(2-trifluoro- |
| | methylphenyl)urea |
| 102 | 2-(4-benzylpiperazin-1-yl)-5-naphthalen-1-ylmethylenethiazol-4-one |
| 103 | 2-(4-benzylpiperazin-1-yl)-5-(6-methoxynaphthalen-2-ylmethylene)thiazol- |
| | 4-one |
| 104 | 2-(4-benzylpiperazin-1-yl)-5-(4-tert-butylbenzylidene)thiazol-4-one |
| 105 | 1-[4-oxo-5-(3-phenylallylidene)-4,5-dihydrothiazol-2-yl]-3-(2-trifluoromethyl- |
| | phenyl)urea |
| 106 | 1-[4-oxo-5-(4-trifluoromethylbenzylidene)-4,5-dihydrothiazol-2-yl]-3- |
| | (4-trifluoromethylphenyl)urea |
| 107 | 1-[5-(4-methylbenzylidene)-4-oxo-4,5-dihydrothiazol-2-yl]-3-(4-trifluoro- |
| | methylphenyl)urea |
| 108 | 5-{3-[4-(6-chloropyridin-2-yl)piperazine-1-carbonyl]benzylidene}-2-thio- |
| | morpholin-4-ylthiazol-4-one |
| 109 | 2-(4-benzylpiperazin-1-yl)-5-(4-trifluoromethoxybenzylidene)thiazol-4-one |
| 110 | 5-{3-[4-(3-chloropyridin-2-yl)-3-methylpiperazine-1-carbonyl]benzylidene}- |
| | 2-thiomorpholin-4-ylthiazol-4-one |
| 111 | N-[3-(3-chloro-5-trifluoromethylpyridin-2-ylamino)propyl]-3-(4-oxo-2- |
| | thiomorpholin-4-yl-4H-thiazol-5-ylidenemethyl)benzamide |
| 112 | 3-(4-oxo-2-thiomorpholin-4-yl-4H-thiazol-5-ylidenemethyl)benzoic acid |
| | N′-(2-chloro-4-trifluoromethylphenyl)hydrazide |
| 113 | 5-{3-[4-(3-chloro-5-trifluoromethylpyridin-2-yl)piperazine-1-carbonyl]- |
| | benzylidene}-2-thiomorpholin-4-ylthiazol-4-one |
| 114 | 3-(4-oxo-2-thiomorpholin-4-yl-4H-thiazol-5-ylidenemethyl)benzoic acid |
| | N′-(3-chloro-5-trifluoromethylpyridin-2-yl)hydrazide |
| 115 | 5-{3-[4-(4-chloro-[1,2,5]thiadiazol-3-yl)piperazine-1-carbonyl]benzylidene}-2- |
| | thiomorpholin-4-ylthiazol-4-one |
| 116 | 3-(4-oxo-2-thiomorpholin-4-yl-4H-thiazol-5-ylidenemethyl)benzoic acid |
| | N′-(2-chloro-5-trifluoromethylphenyl)hydrazide |
| 117 | 2-[4-(4-chloro-[1,2,5]thiadiazol-3-yl)piperazin-1-yl]-5-(4-methylbenzylidene)- |
| | thiazol-4-one |
| 118 | 2-[4-(4-chloro-[1,2,5]thiadiazol-3-yl)piperazin-1-yl]-5-(4-trifluoromethyl- |
| | benzylidene)thiazol-4-one |
| 119 | 2-[4-(4-chloro-[1,2,5]thiadiazol-3-yl)piperazin-1-yl]-5-(4-trifluoromethoxy- |
| | benzylidene)thiazol-4-one |
| 120 | 5-(4-tert-butylbenzylidene)-2-[4-(4-chloro-[1,2,5]thiadiazol-3-yl)piperazin- |
| | 1-yl]thiazol-4-one |
| 121 | 2-[4-(4-chloro-[1,2,5]thiadiazol-3-yl)piperazin-1-yl]-5-(3-phenylallylidene)-thiazol-4-one |
| 122 | 5-[3-(4-tert-butylphenyl)allylidene]-2-[4-(4-chloro-[1,2,5]thiadiazol-3-yl)- |
| | piperazin-1-yl]thiazol-4-one |
| 123 | 2-[4-(4-chloro-[1,2,5]thiadiazol-3-yl)piperazin-1-yl]-5-naphthalen-1-yl- |
| | methylenethiazol-4-one |
| 124 | 2-[4-(4-chloro-[1,2,5]thiadiazol-3-yl)piperazin-1-yl]-5-(3-phenoxy- |
| | benzylidene)thiazol-4-one |
| 125 | 2-[4-(4-chloro-[1,2,5]thiadiazol-3-yl)piperazin-1-yl]-5-(4-phenoxy- |
| | benzylidene)thiazol-4-one |
| 126 | 5-(3-benzyloxybenzylidene)-2-[4-(4-chloro-[1,2,5]thiadiazol-3-yl)piperazin- |
| | 1-yl]thiazol-4-one |
| 127 | 2-[4-(4-chloro-[1,2,5]thiadiazol-3-yl)piperazin-1-yl]-5-(4-hydroxy-3-methoxy- |
| | benzylidene)thiazol-4-one |
| 128 | 2-[4-(4-chloro-[1,2,5]thiadiazol-3-yl)piperazin-1-yl]-5-furan-2-yl- |
| | methylenethiazol-4-one |
| 129 | 2-[4-(4-chloro-[1,2,5]thiadiazol-3-yl)piperazin-1-yl]-5-naphthalen-2-yl- |
| | methylenethiazol-4-one |
| 130 | 3-[2-(4-benzylpiperazin-1-yl)-4-oxo-4H-thiazol-5-ylidenemethyl]benzoic acid |
| 131 | 5-naphthalen-1-ylmethylene-2-piperazin-1-ylthiazol-4-one |
| 132 | 2-piperazin-1-yl-5-(4-trifluoromethylbenzylidene)thiazol-4-one |
| 133 | 5-(4-methylbenzylidene)-2-piperazin-1-ylthiazol-4-one |
| 134 | 5-(4-hydroxy-3-methoxybenzylidene)-2-piperazin-1-ylthiazol-4-one |
| 135 | 5-(4-isopropylbenzylidene)-2-morpholin-4-ylthiazol-4-one |
| 136 | 5-(4-isopropylbenzylidene)-2-thiomorpholin-4-ylthiazol-4-one |
| 137 | 2-[4-(2-fluorophenyl)piperazin-1-yl]-5-(4-isopropylbenzylidene)thiazol-4-one |
| 138 | 5-(4-pentafluorosulfanyl)benzylidene)-2-thiomorpholin-4-ylthiazol-4-one |
| 139 | 2-[4-(3-chloropyridin-2-yl)piperazin-1-yl]-5-(4-isopropylbenzylidene)thiazol-4- |
| | one |
| 140 | 2-(4-benzylpiperazin-1-yl)-5-(4-butylbenzylidene)thiazol-4-one hydrochloride |
| 141 | 2-(4-benzylpiperazin-1-yl)-5-(4-pentylbenzylidene)thiazol-4-one |
| | hydrochloride |
| 142 | 2-(4-benzylpiperazin-1-yl)-5-(4-octylbenzylidene)thiazol-4-one |
| 143 | 2-[4-(6-chloropyridin-2-yl)piperazin-1-yl]-5-(4-methylbenzylidene)thiazol- |
| | 4-one |
| 144 | 5-(4-tert-butylbenzylidene)-2-[4-(6-chloropyridin-2-yl)piperazin-1-yl]thiazol-4- |
| | one |
| 145 | 2-[4-(6-chloropyridin-2-yl)piperazin-1-yl]-5-(4-trifluoromethylbenzylidene)- |
| | thiazol-4-one |
| 146 | 2-[4-(6-chloropyridin-2-yl)piperazin-1-yl]-5-(3-phenylallylidene)thiazol-4-one |
| 147 | 5-[3-(4-tert-butylphenyl)allylidene]-2-[4-(6-chloropyridin-2-yl)piperazin-1-yl]- |
| | thiazol-4-one |
| 148 | 2-[4-(6-chloropyridin-2-yl)piperazin-1-yl]-5-(4-hydroxy-3-methoxy- |
| | benzylidene)thiazol-4-one |
| 149 | 2-[4-(6-chloropyridin-2-yl)piperazin-1-yl]-5-(4-isopropylbenzylidene)thiazol-4- |
| | one |
| 150 | 2-[4-(6-chloropyridin-2-yl)piperazin-1-yl]-5-(4-trifluoromethoxybenzylidene)- |
| | thiazol-4-one |
| 151 | 2-[4-(6-chloropyridin-2-yl)piperazin-1-yl]-5-naphthalen-2-ylmethylenethiazol- |
| | 4-one |
| 152 | 5-(4-methylbenzylidene)-2-[4-(6-methylpyridazin-3-yl)piperazin-1-yl]thiazol- |
| | 4-one |
| 153 | 5-(4-isopropylbenzylidene)-2-[4-(6-methylpyridazin-3-yl)piperazin-1-yl]- |
| | thiazol-4-one |
| 154 | 5-(4-tert-butylbenzylidene)-2-[4-(6-methylpyridazin-3-yl)piperazin-1-yl]- |
| | thiazol-4-one |
| 155 | 5-[3-(4-tert-butylphenyl)allylidene]-2-[4-(6-methylpyridazin-3-yl)piperazin- |
| | 1-yl]thiazol-4-one |
| 156 | 2-[4-(6-methylpyridazin-3-yl)piperazin-1-yl]-5-(4-trifluoromethoxy- |
| | benzylidene)thiazol-4-one |
| 157 | 5-(4-hydroxy-3-methoxybenzylidene)-2-[4-(6-methylpyridazin-3-yl)piperazin- |
| | 1-yl]thiazol-4-one |
| 158 | 2-(4-benzylpiperazin-1-yl)-5-(4-pentafluorosulfanylbenzylidene)thiazol-4-one |
| 159 | 2-(4-benzylpiperazin-1-yl)-5-{3-[4-(4-chloro-[1,2,5]thiadiazol-3-yl)piperazine- |
| | 1-carbonyl]benzylidene}thiazol-4-one |
| 160 | 2-[4-(6-methylpyridazin-3-yl)piperazin-1-yl]-5-(4-trifluoromethylbenzylidene)- |
| | thiazol-4-one |
| 161 | 2-[4-(6-methylpyridazin-3-yl)piperazin-1-yl]-5-(3-phenylallylidene)thiazol- |
| | 4-one |
| 162 | 2-[4-(6-methylpyridazin-3-yl)piperazin-1-yl]-5-naphthalen-2-ylmethylene- |
| | thiazol-4-one |
| 163 | 2-[4-(6-methylpyridazin-3-yl)piperazin-1-yl]-5-naphthalen-1-ylmethylene- |
| | thiazol-4-one |
| 164 | 2-(4-benzylpiperazin-1-yl)-5-(4-isopropylbenzylidene)thiazol-4-one |
| | hydrochloride |
| 165 | 3-{2-[4-(4-chloro-[1,2,5]thiadiazol-3-yl)piperazin-1-yl]-4-oxo-4H-thiazol- |
| | 5-ylidenemethyl}benzoic acid |
| 166 | 5-{3-[4-(4-chloro-3-trifluoromethylphenyl)-4-hydroxypiperidine-1-carbonyl]- |
| | benzylidene}-2-thiomorpholin-4-ylthiazol-4-one |
| 167 | 5-{3-[4-(4-chloro-[1,2,5]thiadiazol-3-yl)piperazine-1-carbonyl]benzylidene}-2- |
| | [4-(4-chloro-[1,2,5]thiadiazol-3-yl)piperazin-1-yl]thiazol-4-one |
| 168 | 5-{3-[4-(3-chloropyridin-2-yl)piperazine-1-carbonyl]benzylidene}-2-[4-(4- |
| | chloro-[1,2,5]thiadiazol-3-yl)piperazin-1-yl]thiazol-4-one |
| 169 | 5-[3-(4-benzylpiperazine-1-carbonyl)benzylidene]-2-[4-(4-chloro- |
| | [1,2,5]thiadiazol-3-yl)piperazin-1-yl]thiazol-4-one |
| 170 | 2-[4-(4-chloro-[1,2,5]thiadiazol-3-yl)piperazin-1-yl]-5-{3-[4-(6-methoxy- |
| | pyridin-2-yl)piperazine-1-carbonyl]benzylidene}thiazol-4-one |
| 171 | 2-[4-(4-chloro-3-trifluoromethylphenyl)-4-hydroxypiperidin-1-yl]-5-(4- |
| | methylbenzylidene)thiazol-4-one |
| 172 | 2-[4-(4-chloro-3-trifluoromethylphenyl)-4-hydroxypiperidin-1-yl]-5-(4-hydroxy- |
| | 3-methoxybenzylidene)thiazol-4-one |
| 173 | 2-[4-(4-chloro-3-trifluoromethylphenyl)-4-hydroxypiperidin-1-yl]-5- |
| | naphthalen-1-ylmethylenethiazol-4-one |
| 174 | 2-[4-(4-chloro-3-trifluoromethylphenyl)-4-hydroxypiperidin-1-yl]-5-(3- |
| | methoxybenzylidene)thiazol-4-one |
| 175 | 5-benzylidene-2-morpholin-4-ylthiazol-4-one hydrobromide |
| 176 | 5-(4-methylbenzylidene)-2-morpholin-4-ylthiazol-4-one hydrobromide |
| 177 | 2-diethylamino-5-(4-methylbenzylidene)thiazol-4-one hydrobromide |
| 178 | 5-(4-chlorobenzylidene)-2-diethylaminothiazol-4-one hydrobromide |
| 179 | 5-(4-isopropylbenzylidene)-2-morpholin-4-ylthiazol-4-one hydrobromide |
| 180 | 5-benzylidene-2-diethylaminothiazol-4-one hydrobromide |
| 181 | 5-(4-chlorobenzylidene)-2-morpholin-4-ylthiazol-4-one hydrobromide |
| 182 | 5-(4-chlorobenzylidene)-2-piperidin-1-ylthiazol-4-one hydrobromide |
| 183 | 5-(4-chlorobenzylidene)-2-pyrrolidin-1-ylthiazol-4-one hydrobromide |
| 184 | 5-benzylidene-2-pyrrolidin-1-ylthiazol-4-one hydrobromide |
| 185 | 5-benzylidene-2-pyrrolidin-1-ylthiazol-4-one |
| 186 | 5-biphenyl-4-ylmethylene-2-piperidin-1-ylthiazol-4-one hydrobromide |
| 187 | 2-azepan-1-yl-5-biphenyl-4-ylmethylenethiazol-4-one hydrobromide |
| 188 | 5-biphenyl-4-ylmethylene-2-pyrrolidin-1-ylthiazol-4-one hydrobromide |
| 189 | 2-morpholin-4-yl-5-(3-phenylallylidene)thiazol-4-one hydrobromide |
| 190 | 5-(3-phenylallylidene)-2-thiomorpholin-4-ylthiazol-4-one hydrobromide |
| 191 | 5-(3-phenylallylidene)-2-piperidin-1-ylthiazol-4-one hydrobromide |
| 192 | 2-azepan-1-yl-5-(3-phenylallylidene)thiazol-4-one hydrobromide |
| 193 | 5-(3-phenylallylidene)-2-pyrrolidin-1-ylthiazol-4-one hydrobromide |
| 194 | 5-biphenyl-4-ylmethylene-2-morpholin-4-ylthiazol-4-one hydrobromide |
| 195 | 2-thiomorpholin-4-yl-5-(3-trifluoromethylbenzylidene)thiazol-4-one |
| | hydrobromide |
| 196 | 5-(4-chlorobenzylidene)-2-thiomorpholin-4-ylthiazol-4-one |
| 197 | 5-(4-tert-butylbenzylidene)-2-morpholin-4-ylthiazol-4-one |
| 198 | 5-(4-tert-butylbenzylidene)-2-thiomorpholin-4-ylthiazol-4-one hydrobromide |
| 199 | 5-(4-tert-butylbenzylidene)-2-pyrrolidin-1-ylthiazol-4-one hydrobromide |
| 200 | 2-azepan-1-yl-5-(4-tert-butylbenzylidene)thiazol-4-one hydrobromide |
| 201 | 5-(4-tert-butylbenzylidene)-2-piperidin-1-ylthiazol-4-one hydrobromide |
| 202 | 2-thiomorpholin-4-yl-5-(4-trifluoromethylbenzylidene)thiazol-4-one |
| 203 | 5-(4-tert-butylbenzylidene)-2-diethylaminothiazol-4-one hydrobromide |
| 204 | 2-morpholin-4-yl-5-(4-trifluoromethylbenzylidene)thiazol-4-one |
| 205 | 2-morpholin-4-yl-5-(3-trifluoromethylbenzylidene)thiazol-4-one hydrobromide |
| 206 | 2-morpholin-4-yl-5-(3-trifluoromethylbenzylidene)thiazol-4-one |
| 207 | 5-biphenyl-4-ylmethylene-2-diethylaminothiazol-4-one |
| 208 | 2-morpholin-4-yl-5-thiophen-2-ylmethylenethiazol-4-one |
| 209 | 2-morpholin-4-yl-5-naphthalen-1-ylmethylenethiazol-4-one |
| 210 | 2-morpholin-4-yl-5-pyridin-2-ylmethylenethiazol-4-one |
| 211 | 2-morpholin-4-yl-5-(4-trifluoromethylbenzylidene)thiazol-4-one |
| 212 | 2-morpholin-4-yl-5-pyridin-3-ylmethylenethiazol-4-one |
| 213 | 2-morpholin-4-yl-5-pyridin-4-ylmethylenethiazol-4-one |
| 214 | 2-diethylamino-5-(3-trifluoromethylbenzylidene)thiazol-4-one |
| 215 | 5-(4-methylbenzylidene)-2-pyrrolidin-1-ylthiazol-4-one |
| 216 | 5-(4-tert-butylbenzylidene)-2-(2-methoxyethylamino)thiazol-4-one |
| | hydrobromide |
| 217 | 2-morpholin-4-yl-5-(4-octylbenzylidene)thiazol-4-one |
| 218 | 5-(4-methylbenzylidene)-2-morpholin-4-ylthiazol-4-one |
| 219 | 5-(3,4-dimethoxybenzylidene)-2-morpholin-4-ylthiazol-4-one |
| 220 | 5-(2-hydroxybenzylidene)-2-morpholin-4-ylthiazol-4-one |
| 221 | 5-(2-hydroxy-3-methoxybenzylidene)-2-morpholin-4-ylthiazol-4-one |
| 222 | 5-(4-tert-butylbenzylidene)-2-morpholin-4-ylthiazol-4-one |
| 223 | 5-(4-diethylaminobenzylidene)-2-morpholin-4-ylthiazol-4-one |
| 224 | 5-(4-hydroxy-3-methoxybenzylidene)-2-morpholin-4-ylthiazol-4-one |
| 225 | 4-(2-morpholin-4-yl-4-oxo-4H-thiazol-5-ylidenemethyl)benzoic acid |
| 226 | 2-morpholin-4-yl-5-(2,3,4-trimethoxybenzylidene)thiazol-4-one |
| 227 | 2-morpholin-4-yl-5-(3,4,5-trimethoxybenzylidene)thiazol-4-one |
| 228 | 5-(4-methoxybenzylidene)-2-morpholin-4-ylthiazol-4-one |
| 229 | 5-(4-hydroxybenzylidene)-2-morpholin-4-ylthiazol-4-one |
| 230 | 5-(3-ethoxy-4-hydroxybenzylidene)-2-morpholin-4-ylthiazol-4-one |
| 231 | 5-(3-hydroxybenzylidene)-2-morpholin-4-ylthiazol-4-one |
| 232 | 5-(4-bromobenzylidene)-2-morpholin-4-ylthiazol-4-one |
| 233 | 2-morpholin-4-yl-5-(4-vinyloxybenzylidene)thiazol-4-one |
| 234 | benzenesulfonic acid 4-(2-morpholin-4-yl-4-oxo-4H-thiazol-5-ylidene- |
| | methyl)phenyl ester |
| 235 | 5-(4-dimethylaminobenzylidene)-2-morpholin-4-ylthiazol-4-one |
| 236 | 4-(2-morpholin-4-yl-4-oxo-4H-thiazol-5-ylidenemethyl)benzoic acid methyl |
| | ester |
| 237 | 5-(3-hydroxy-4-methoxybenzylidene)-2-morpholin-4-ylthiazol-4-one |
| 238 | 5-[4-(3,3-dimethylbutoxy)-3-methoxybenzylidene]-2-morpholin-4-ylthiazol- |
| | 4-one |
| 239 | 5-(2-methoxybenzylidene)-2-morpholin-4-ylthiazol-4-one |
| 240 | 5-(4-hydroxybenzylidene)-2-morpholin-4-ylthiazol-4-one |
| 241 | 2-[(benzo[1,3]dioxol-5-ylmethyl)amino]-5-[3-(4-tert-butylphenyl)- |
| | allylidene]thiazol-4-one |
| 242 | 2-[(benzo[1,3]dioxol-5-ylmethyl)amino]-5-(4-trifluoromethylbenzylidene)- |
| | thiazol-4-one |
| 243 | 2-[(benzo[1,3]dioxol-5-ylmethyl)amino]-5-(4-trifluoromethoxybenzylidene)- |
| | thiazol-4-one |
| 244 | 2-[(benzo[1,3]dioxol-5-ylmethyl)amino]-5-naphthalen-1-ylmethylenethiazol- |
| | 4-one |
| 245 | 2-[(benzo[1,3]dioxol-5-ylmethyl)amino]-5-(2-chloro-3-phenylallylidene)- |
| | thiazol-4-one |
| 246 | 2-(4-methylbenzylamino)-5-naphthalen-1-ylmethylenethiazol-4-one |
| 247 | 2-(4-methylbenzylamino)-5-(3-phenoxybenzylidene)thiazol-4-one |
| 248 | 2-(4-methylbenzylamino)-5-(4-phenoxybenzylidene)thiazol-4-one |
| 249 | 5-(2-chloro-3-phenylallylidene)-2-(4-methylbenzylamino)thiazol-4-one |
| 250 | 2-(4-methylbenzylamino)-5-naphthalen-2-ylmethylenethiazol-4-one |
| 251 | 2-(3-methoxybenzylamino)-5-(4-methylbenzylidene)thiazol-4-one |
| 252 | 5-(4-tert-butylbenzylidene)-2-(3-methoxybenzylamino)thiazol-4-one |
| 253 | 2-(3-methoxybenzylamino)-5-(4-trifluoromethylbenzylidene)thiazol-4-one |
| 254 | 2-(3-methoxybenzylamino)-5-(4-trifluoromethoxybenzylidene)thiazol-4-one |
| 255 | 2-(3-methoxybenzylamino)-5-(3-phenylallylidene)thiazol-4-one |
| 256 | 5-naphthalen-1-ylmethylene-2-(4-trifluoromethylbenzylamino)thiazol-4-one |
| 257 | 5-(3-phenoxybenzylidene)-2-(4-trifluoromethylbenzylamino)thiazol-4-one |
| 258 | 5-(4-phenoxybenzylidene)-2-(4-trifluoromethylbenzylamino)thiazol-4-one |
| 259 | 5-(2-chloro-3-phenylallylidene)-2-(4-trifluoromethylbenzylamino)thiazol- |
| | 4-one |
| 260 | 5-(3-benzyloxybenzylidene)-2-(4-trifluoromethylbenzylamino)thiazol-4-one |
| 261 | 5-naphthalen-2-ylmethylene-2-(4-trifluoromethylbenzylamino)thiazol-4-one |

### Pharmacological Data

The affinity of the inventive 2,5-disubstituted thiazol-4-one derivatives for vanilloid receptor 1 (VR1/TRPV1 receptor) was determined as described above.

| | VR1 (rat) | VR1 (rat) | VR1 (human) | VR1 (human) | | | | |
|---|---|---|---|---|---|---|---|---|
| Compound | (% stimulation | (% inhibition | (% stimulation | (% inhibition | EC₅₀ | EC₅₀ | IC₅₀ | IC₅₀ |
| according | compared to | compared to | compared to | compared to | VR1 | VR1 | VR1 | VR1 |
| to example | 10 μM CP) | 10 μM CP) | 10 μM CP) | 10 μM CP) | (rat) | (human) | (rat) | (human) |
| 12 | 53.99 | 68.39 | 55.40 | 54.33 | | | | |
| 13 | 42.13 | 46.23 | 6.15 | 11.13 | | | | |
| 14 | 107.63 | 82.14 | 106.96 | 89.44 | 0.95 | 5.17 | | |
| 17 | 119.55 | 91.14 | 45.99 | 19.65 | 6.36 | 17.78 | | |
| 18 | 144.22 | 88.01 | 139.33 | 79.17 | 1.295 | 3.645 | | |
| 19 | 11.71 | 31.38 | 8.82 | 10.83 | 7.755 | 13.02 | | |
| 21 | 55.77 | 50.12 | 36.52 | 28.51 | 6.595 | 8.51 | | |
| 22 | 116.13 | 88.08 | 63.95 | 80.79 | 8.485 | 9.06 | | |
| 24 | 110.20 | 91.35 | 61.33 | 61.63 | 11.875 | >25 | | |
| 25 | 80.77 | 87.21 | 29.07 | 37.65 | | | | |
| 30 | 0.92 | 97.01 | 0.06 | 73.26 | | | >50 | >100 |
| 33 | 49.60 | 64.80 | 25.74 | 14.65 | | | | |
| 34 | 100.54 | 87.96 | 110.57 | 89.29 | | | | |
| 36 | 67.65 | 100.59 | 47.43 | 68.80 | | | | |
| 39 | 31.17 | 50.97 | 18.89 | 23.67 | | | | |
| 41 | 86.95 | 90.93 | 0.47 | 22.60 | 5.82 | 10.56 | | |
| 42 | 98.33 | 93.08 | 49.72 | 50.18 | | | | |
| 44 | 39.61 | 64.34 | 10.95 | 34.31 | | | | |
| 49 | 0.40 | 78.48 | 0.00 | 34.15 | | | >50 | >50 |
| 50 | 7.19 | 21.27 | 0.00 | 51.62 | | | | |
| 54 | 96.08 | 87.88 | 79.60 | 81.73 | 2.5 | >25 | | |
| 63 | 0.34 | 70.90 | 0.00 | 52.80 | | | 3.04 | 9.5 |
| 68 | 73.37 | 11.64 | 54.28 | 39.34 | | | | |
| 85 | 54.91 | 77.11 | 82.46 | 97.47 | | | | |
| 88 | 72.85 | 90.08 | 61.42 | 71.14 | | | | |
| 96 | 28.48 | 26.98 | 5.03 | 54.65 | | | | |
| 110 | 49.33 | 50.28 | 1.02 | 28.75 | | | | |
| 113 | 135.99 | 97.39 | 90.31 | 97.83 | | | | |
| 115 | 128.30 | 95.77 | 68.25 | 66.21 | | | | |
| 135 | 79.68 | 87.60 | 85.02 | 84.29 | | | | |
| 136 | 83.98 | 96.93 | 114.42 | 86.67 | | | | |
| 154 | 3.99 | 54.11 | 6.56 | 34.23 | | | | |
| 161 | 2.50 | 17.01 | 2.43 | 47.22 | | | | |
| 176 | 44.68 | 97.55 | 14.52 | 35.32 | | | | |
| 179 | 41.89 | 38.36 | 5.04 | 30.72 | | | | |
| 189 | 6.64 | 5.61 | 11.15 | 8.14 | >25 | >50 | | |
| 190 | 51.35 | 83.13 | 34.09 | 32.35 | 14.27 | >25 | | |
| 191 | −0.17 | 17.65 | 15.81 | 8.04 | >25 | >50 | | |
| 192 | 1.34 | 2.18 | 16.72 | 10.31 | >50 | >50 | | |
| 195 | 94.27 | 96.09 | 80.31 | 90.32 | 2.2 | 5.9 | | |
| 197 | 63.86 | 94.35 | 48.05 | 72.42 | | | | |
| 198 | 60.71 | 97.57 | 54.31 | 76.67 | 1.12 | 1.02 | | |
| 199 | 42.91 | 69.55 | 9.64 | 4.43 | | | | |
| 200 | 31.45 | 46.20 | 17.52 | 2.12 | >25 | >25 | | |
| 205 | 79.60 | 96.40 | 68.23 | 102.91 | | | | |
| 206 | 76.51 | 102.30 | 77.26 | 95.06 | | | | |
| 209 | 39.99 | 34.68 | 9.44 | 5.47 | 14.83 | 28.4 | | |
| 210 | 2.22 | 23.01 | 1.83 | 3.03 | | | | |
| 214 | 13.70 | 50.09 | 6.08 | 56.74 | | | | |
| 215 | 2.14 | 4.47 | 3.64 | 49.80 | | | | |
| 216 | 1.24 | 22.13 | 2.08 | 51.07 | 1.5 | 5.4 | | |
| 218 | 55.76 | 78.83 | 17.91 | 12.88 | | | | |
| 222 | 102.46 | 98.64 | 41.92 | 79.67 | | | | |
| 223 | 101.77 | 9.30 | 70.53 | 57.72 | | | | |
| 228 | 16.03 | 4.84 | 12.18 | 0.61 | 14.3 | 26.5 | | |
| 232 | 80.44 | 96.18 | 29.19 | 43.80 | | | | |
| 234 | 57.90 | 98.08 | 46.10 | 27.80 | | | | |
| 235 | 123.39 | 13.66 | 73.87 | 44.50 | | | | |

1-34. (canceled)

35. A method of treating or inhibiting pain in a subject, said method comprising administering to said subject a pharmacologically effective amount of a 2,5-disubstituted thiazol-4-one compound corresponding to formula I, 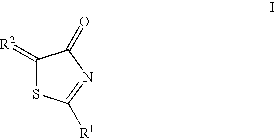
wherein
R¹ is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
is an unsubstituted or at least monosubstituted aryl radical which may be fused to an unsubstituted or at least monosubstituted mono- or polycyclic ring system;
is an NR³R⁴ group;
is an NR⁵—C(═O)—R⁶ group;
or is an NR⁷—C(═O)—NR⁸R⁹ group;
R² is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
is CH—U—X;
is CH—CH₂—V—Y, CH—CH(CH₃)—V—Y, CH—CHCl—V—Y, CH—CHBr—V—Y, CH—CHF—V—Y or CH—CH(OH)—V—Y;
or is CH—CH═C(CH₃)—W-Z, CH—CH═CH—W-Z, CH—C(CH₃)═CH—W-Z, CH—C(phenyl)=CH—W-Z, CH—CBr═CH—W-Z, CH—CCl═CH—W-Z, CH—CF═CH—W-Z, CH—C(OH)═CH—W-Z, CH—CH₂—CH₂—W-Z, CH—CH₂—CH(CH₃)—W-Z or CH—CH(CH₃)—CH₂—W-Z;
where U, V and W may each be absent or are each independently a radical selected from the group consisting of O, S, N(H), N(CH₃), N(C₂H₅) and N[CH(CH₃)₂];
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂;
is a radical selected from the group consisting of cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, thiomorpholinyl and azepanyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl;
where the cyclic moiety of the —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl and —N[CH(CH₃)₂]-pyridinyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, I, —SF₅, —OH, —O—CH₃, —O—C₂H₅, —NH₂, —NO₂, —CF₃, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂ and —NH—C(CH₃)₃;
R⁴ is a hydrogen radical
or is a linear or branched, saturated or unsaturated, unsubstituted or at least monosubstituted aliphatic radical optionally having at least one heteroatom as a chain member;
R⁵, R⁷ and R⁹ are each independently
a hydrogen radical
or a linear or branched, saturated or unsaturated, unsubstituted or at least monosubstituted aliphatic radical;
R⁶ and R⁸ are each independently
an unsubstituted or at least monosubstituted aryl or heteroaryl radical which may be fused to an unsubstituted or at least monosubstituted mono- or polycyclic ring system;
X is an aryl or heteroaryl radical which may be fused to an unsubstituted or at least monosubstituted mono- or polycyclic ring system and/or may be substituted by identical or different substituents R¹⁰;
Y is an aryl or heteroaryl radical which may be fused to an unsubstituted or at least monosubstituted mono- or polycyclic ring system and/or may be substituted by identical or different substituents R¹¹;
Z is a linear or branched, saturated or unsaturated, unsubstituted or at least monosubstituted aliphatic radical;
or is an aryl or heteroaryl radical which may be fused to an unsubstituted or at least monosubstituted mono- or polycyclic ring system and/or may be substituted by identical or different substituents R¹²;
R¹⁰, R¹¹ and R¹² are each independently
a radical selected from the group consisting of F, Cl, Br, I, —CN, —CF₃, —SF₅, —OH, —O—C₁₋₁₀-alkyl, —O—C₂₋₁₀-alkenyl, —NH₂, —NO₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted;
is a —C(═O)—NR¹³—(CH₂)ₘ—NR¹⁴—R¹⁵ group where m is 0, 1, 2, 3, 4 or 5;
or is a —C(═O)—R¹⁶ group;
R¹³ and R¹⁴ are each independently
a hydrogen radical
or a linear or branched, saturated or unsaturated, unsubstituted or at least monosubstituted aliphatic radical;
R¹⁵ is an unsubstituted or at least monosubstituted aryl or heteroaryl radical which may be fused to an unsubstituted or at least monosubstituted mono- or polycyclic ring system; and
R¹⁶ is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
or a pharmaceutically acceptable salt or solvate thereof.

36. A method as claimed in claim 35, wherein
R¹ is an unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
is an optionally substituted 6- or 10-membered aryl radical which may be fused to a saturated or unsaturated, optionally substituted mono- or polycyclic ring system;
is an NR³R⁴ group;
is an NR⁵—C(═O)—R⁶ group
or is an NR⁷—C(═O)—NR⁸R⁹ group;
R² is a unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
is CH—U—X;
is CH—CH₂—V—Y, CH—CH(CH₃)—V—Y, CH—CHCl—V—Y, CH—CHBr—V—Y, CH—CHF—V—Y or CH—CH(OH)—V—Y;
or is CH—CH═C(CH₃)—W-Z, CH—CH═CH—W-Z, CH—C(CH₃)═CH—W-Z, CH—C(phenyl)=CH—W-Z, CH—CBr═CH—W-Z, CH—CCl═CH—W-Z, CH—CF═CH—W-Z, CH—C(OH)═CH—W-Z, CH—CH₂—CH₂—W-Z, CH—CH₂—CH(CH₃)—W-Z or CH—CH(CH₃)—CH₂—W-Z;
where U, V and W may in each case be absent or are in each case independently a radical selected from the group consisting of O, S, N(H), N(CH₃), N(C₂H₅) and N[CH(CH₃)₂];
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂;
is a radical selected from the group consisting of cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, thiomorpholinyl and azepanyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl;
where the cyclic moiety of the —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl and —N[CH(CH₃)₂]-pyridinyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, I, —SF₅, —OH, —O—CH₃, —O—C₂H₅, —NH₂, —NO₂, —CF₃, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂ and —NH—C(CH₃)₃;
R⁴ is a hydrogen radical
or is a linear or branched, saturated or unsaturated, optionally substituted C₁₋₁₀ aliphatic radical optionally having 1, 2 or 3 heteroatoms selected independently from the group consisting of oxygen, nitrogen (NH) and sulfur;
R⁵, R⁷ and R⁸ are each independently
a hydrogen radical
or a linear or branched, saturated or unsaturated, optionally substituted C₁₋₁₀ aliphatic radical;
R⁶ and R⁹ are each independently
an optionally substituted 5- to 14-membered aryl or heteroaryl radical which may be fused to a saturated or unsaturated, optionally substituted mono- or polycyclic ring system;
X is a 5- to 14-membered aryl or heteroaryl radical which may be fused to a saturated or unsaturated, optionally substituted mono- or polycyclic ring system and/or may optionally be substituted by 1, 2, 3, 4 or 5 identical or different substituents R¹⁰;
Y or is an optionally substituted 5- to 14-membered aryl or heteroaryl radical which may be fused to a saturated or unsaturated, optionally substituted mono- or polycyclic ring system and/or may optionally be substituted by 1, 2, 3, 4 or 5 identical or different substituents R¹¹;
Z is a linear or branched, saturated or unsaturated, optionally substituted C₁₋₂₀ aliphatic radical;
or is an optionally substituted 5- to 14-membered aryl or heteroaryl radical which may be fused to a saturated or unsaturated, optionally substituted mono- or polycyclic system and/or may optionally be substituted by 1, 2, 3, 4 or 5 identical or different substituents R¹²;
R¹⁰, R¹¹ and R¹² are each independently
a radical selected from the group consisting of F, Cl, Br, I, —CN, —CF₃, —SF₅, —OH, —O—C₁₋₁₀-alkyl, —O—C₂₋₁₀-alkenyl, —NH₂, —NO₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
a —C(═O)—NR¹³—(CH₂)ₘ—NR¹⁴—R¹⁵ group where m is 0, 1, 2, 3, 4 or 5;
or a —C(═O)—R¹⁶ group;
R¹³ and R¹⁴ are each independently
a hydrogen radical
or is a linear or branched, saturated or unsaturated, optionally substituted C₁₋₁₀ aliphatic radical;
R¹⁵ is a 5- to 14-membered aryl or heteroaryl radical which may be fused to a saturated or unsaturated, optionally substituted mono- or polycyclic ring system; and
R¹⁶ is a unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
where
the aforementioned cycloaliphatic radicals may each optionally be substituted by 1, 2, 3, 4 or 5 substituents selected independently from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—Cl-s-alkyl, —O—C(═O)—C₁₋₅-alkyl, —NH—C₁₋₅-alkyl, —N(C₁₋₅-alkyl)₂, —NH-phenyl, —NH-pyridinyl, —N(C₁₋₅-alkyl)-phenyl, —N(C₁₋₅-alkyl)-pyridinyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, [1,2,5]-thiadiazolyl, pyridazinyl, —(CH₂)-benzo[b]furanyl, —O-phenyl, —O-benzyl, phenyl and benzyl, where the cyclic moiety of the —NH-phenyl, —NH-pyridinyl, —N(C₁₋₅-alkyl)-phenyl, —N(C₁₋₅-alkyl)-pyridinyl, pyridinyl, cyclopentyl, [1,2,5]-thiadiazolyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl,
and the aforementioned cycloaliphatic radicals may each optionally have 1, 2, 3, 4 or 5 heteroatom(s) selected independently from the group consisting of oxygen, nitrogen and sulfur as ring member(s);
the rings of the aforementioned mono- or polycyclic ring systems may each optionally be substituted by 1, 2, 3, 4 or 5 substituents selected independently from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl, where the cyclic moiety of the pyridinyl, cyclopentyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl,
and the rings of the aforementioned mono- or polycyclic ring systems are each 5-, 6- or 7-membered and may each optionally have 1, 2, 3, 4 or 5 heteroatom(s) as ring member(s) which are selected independently from the group consisting of oxygen, nitrogen and sulfur; the aforementioned C₁₋₁₀ aliphatic radicals or C₁₋₂₀ aliphatic radicals may each optionally be substituted by 1, 2, 3, 4, 5, 6, 7, 8 or 9 substituents selected independently from the group consisting of F, Cl, Br, I, —CN, —NO₂, —OH, —SH and —NH₂;
the aforementioned aryl or heteroaryl radicals may each optionally be substituted by 1, 2, 3, 4 or 5 substituents selected independently from the group consisting of F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —NH—C(═O)—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl, where the cyclic moiety of the pyridinyl, cyclopentyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl,
and the aforementioned heteroaryl radicals may each optionally have 1, 2, 3, 4 or 5 heteroatom(s) selected independently from the group consisting of oxygen, nitrogen and sulfur as ring member(s);
and the aforementioned C₁₋₅-alkylene groups may each optionally be substituted by 1, 2, 3, 4 or 5 substituents selected independently from the group consisting of F, Cl, Br, —OH, —SH, —NH₂, —CN and NO₂.

37. A method as claimed in claim 35, wherein
R¹ is a radical selected from the group consisting of cyclopentyl cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, thiomorpholinyl, azepanyl, (2,3)-dihydro-1H-isoindolyl, (2,3)-dihydroindolyl, (2,3,4,9)-tetrahydro-1H-β-carbolinyl, (2,3,4,9)-tetrahydro-1H-pyrido[2,3-b]indolyl, [1,2,3,4]-tetrahydronaphthyl, [1,2,3,4]-tetrahydroquinolinyl, [1,2,3,4]-tetrahydroisoquinolinyl, [1,2,3,4]-tetrahydroquinazolinyl and [3,4]-dihydro-2H-1,4-benzoxazinyl; where the radical may in each case optionally be substituted by 1, 2, 3, 4 or 5 substituents selected independently from the group consisting of oxo (═O), thioxo (═S), F, Cl, Br, I, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl, —NH—C(═O)—O—CH₃, —NH—C(═O)—O—C₂H₅, —NH—C(═O)—O—C(CH₃)₃, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl and phenyl; where the cyclic moiety of the —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl, pyridinyl, pyridazinyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
is a piperazinyl radical which may be substituted by 1, 2 or 3 substituents selected independently from the group consisting of methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —C(═O)—C₁₋₅-alkyl, —C(═O)—NH₂, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
is a radical selected from the group consisting of phenyl and naphthyl, where the radical may in each case optionally be substituted by 1, 2, 3, 4 or 5 substituents selected independently from the group consisting of F, Cl, Br, I, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂ and —NH—C(CH₃)₃;
is an NR³R⁴ group;
is an NR⁵—C(═O)—R⁶ group
or is an NR⁷—C(═O)—NR⁸R⁹ group.

38. A method as claimed in claim 35, wherein
R² is a 1,3-dihydroindol-2-onyl or a 1,3-dihydroindol-2-thionyl radical;
is CH—X;
is CH—CH₂—Y, CH—CH(CH₃)—Y, CH—CHCl—Y, CH—CHBr—Y, CH—CHF—Y or CH—CH(OH)—Y;
or is CH—CH═C(CH₃)-Z, CH—CH═CH-Z, CH—CH═CH—S-Z, CH—CH═CH—O-Z, CH—CH═CH—N(CH₃)-Z, CH—C(CH₃)═CH-Z, CH—C(phenyl)═CH-Z, CH—CBr═CH-Z, CH—CCl═CH-Z, CH—CF═CH-Z, CH—C(OH)═CH-Z, CH—CH₂—CH₂-Z, CH—CH₂—CH(CH₃)-Z or CH—CH(CH₃)—CH₂-Z.

39. A method as claimed in claim 35, wherein
R⁴ is a hydrogen radical
or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂.

40. A method as claimed in claim 35, wherein
R⁵, R⁷ and R⁸ are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂.

41. A method as claimed in claim 35, wherein
R⁶ and R⁹ are each independently
a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃.

42. A method as claimed in claim 35, wherein
X is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R¹⁰.

43. A method as claimed in claim 35, wherein
Y is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R¹¹.

44. A method as claimed in claim 35, wherein
Z is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl, where the radical may in each case optionally be substituted by 1, 2, 3, 4 or 5 substituents selected independently from the group consisting of F, Cl, Br, I, —CN, —NO₂, —OH, —SH and —NH₂;
or is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R¹².

45. A method as claimed in claim 35, wherein
R¹⁰, R¹¹ and R¹² are each independently
a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —NH—C(═O)—CH₃, —NH—C(═O)—C₂H₅, —NH—C(═O)—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —NH—S(═O)₂—CH₃, —NH—S(═O)₂—C₂H₅, —S(═O)₂—NH—CH₃, —S(═O)₂-phenyl, —O—S(═O)₂-phenyl, cyclohexyl, cyclopentyl, pyridinyl, pyridazinyl, —(CH₂)— benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂ methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a —C(═O)—NR¹³—(CH₂)ₘ—NR¹⁴—R¹⁵ group where m is 0, 1, 2 or 3;
or a —C(═O)—R¹⁶ group;
wherein
R¹³ and R¹⁴ are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R¹⁵ is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —N—H—C(═O)—O—C(CH₃)₃, —C(═O)—H, —C(═O)—CH₃, —C(═O)—C₂H₅, —C(═O)—C(CH₃)₃; and
R¹⁶ is a radical selected from the group consisting of piperazinyl, thiomorpholinyl, azepanyl, morpholinyl, 2,3-dihydro-1H-isoindolyl, 2,3-dihydroindolyl, piperidinyl and pyrrolidinyl; where the radical may be substituted by 1, 2 or 3 substituents selected independently from the group consisting of —OH, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —C(═O)—C₁₋₅-alkyl, —C(═O)—NH₂, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

46. A method as claimed in claim 35, wherein
R¹ is one of the following radicals 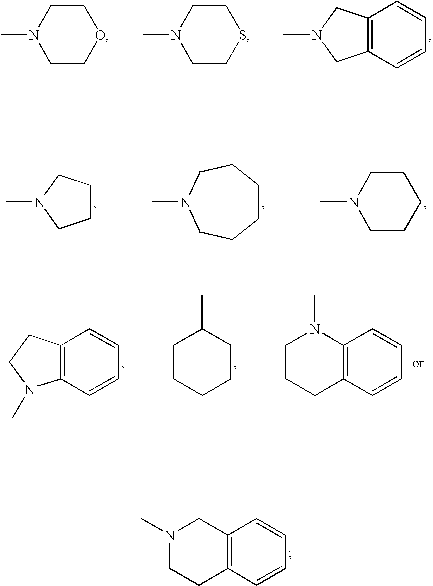
where the radical may be substituted by 1, 2 or 3 substituents selected independently from the group consisting of —OH, —O—CH₃, —O—C₂H₅, methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)— phenyl, —N(C₂H₅)-phenyl, —N[CH(CH₃)₂]-phenyl, —N(CH₃)-pyridinyl, —N(C₂H₅)— pyridinyl, —N[CH(CH₃)₂]-pyridinyl and phenyl; where the cyclic moiety of the phenyl radical may in each case be substituted by 1, 2 or 3 substituents selected independently from the group consisting of F, Cl, Br and —CF₃;
is one of the following radicals 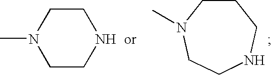
where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is a radical selected from the group consisting of phenyl and naphthyl, where the radical may in each case optionally be substituted by 1, 2, 3, 4 or 5 substituents selected independently from the group consisting of —OH, —O—CH₃ and —O—C₂H₅;
is an NR³R⁴ group
or is an NR⁷—C(═O)—NR⁸R⁹ group;
R² is a 1,3-dihydroindol-2-onyl or a 1,3-dihydroindol-2-thionyl radical;
is CH—X;
is CH—CH₂—Y, CH—CH(CH₃)—Y, CH—CHCl—Y, CH—CHBr—Y, CH—CHF—Y or CH—CH(OH)—Y;
or is CH—CH═C(CH₃)-Z, CH—CH═CH-Z, CH—CH═CH—S-Z, CH—CH═CH—O-Z, CH—CH═CH—N(CH₃)-Z, CH—C(CH₃)═CH-Z, CH—C(phenyl)=CH-Z, CH—CBr═CH-Z, CH—CCl═CH-Z, CH—CF═CH-Z, CH—C(OH)═CH-Z, CH—CH₂—CH₂-Z, CH—CH₂—CH(CH₃)-Z or CH—CH(CH₃)—CH₂-Z;
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
is a radical selected from the group consisting of cyclopentyl, cyclohexyl, cycloheptyl, cyclopentenyl, cyclohexenyl and cycloheptenyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl;
or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R⁴ is a hydrogen radical
or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁷ and R⁸ are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁹ is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CH₃, —O—C₂H₅, —O—CF₃, —S—CF₃, methyl, ethyl, n-propyl, isopropyl, n-butyl, sec-butyl, isobutyl and tert-butyl;
X is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹⁰;
Y is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹¹;
Z is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl;
or is a radical selected from the group consisting of phenyl, naphthyl, phenanthrenyl, (1,3)-benzodioxolyl, (1,4)-benzodioxanyl, thiophenyl, furanyl, pyrrolyl, pyrazolyl, pyrazinyl, pyridinyl, pyridazinyl, pyrimidinyl, quinolinyl and isoquinolinyl; which may optionally be substituted by 1, 2, 3, 4 or 5 identical or different substituents R¹²;
R¹⁰ is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—CH₃, —NH—C(═O)—C₂H₅, —NH—C(═O)—C(CH₃)₃, —O—S(═O)₂-phenyl, —O-phenyl, —O-benzyl, phenyl and benzyl;
is a —C(═O)—NR¹³—(CH₂)ₘ—NR¹⁴—R⁵ group where m is 0, 1, 2 or 3;
or is a —C(═O)—R¹⁶ group;
R¹¹ is a radical selected from the group consisting of F, Cl, Br, —SF₅, methyl, ethyl, n-propyl, isopropyl, n-butyl, sec-butyl, tert-butyl, n-pentyl, n-hexyl, n-heptyl and n-octyl;
R¹² is a radical selected from the group consisting of F, Cl, Br, —SF₅, —CF₃, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, methyl, ethyl, n-propyl, isopropyl, n-butyl, sec-butyl, tert-butyl, n-pentyl, n-hexyl, n-heptyl and n-octyl;
R¹³ and R¹⁴ are each a hydrogen radical;
R¹⁵ is a radical selected from the group consisting of phenyl and pyridinyl, where the radical may in each case optionally be substituted by 1, 2, 3, 4 or 5 substituents selected independently from the group consisting of F, Cl, Br, —CF₃, —O—CF₃, —S—CF₃, methyl, ethyl, n-propyl, isopropyl, n-butyl, sec-butyl, isobutyl and tert-butyl; and
R¹⁶ is a radical selected from the group consisting of thiomorpholinyl, azepanyl, morpholinyl, (2,3)-dihydro-1H-isoindolyl, (2,3)-dihydroindolyl, piperidinyl and pyrrolidinyl; where the radical may be substituted by 1, 2 or 3 substituents selected independently from the group consisting of —OH, methyl, ethyl, n-propyl, isopropyl, n-butyl, sec-butyl, isobutyl, tert-butyl, benzyl and phenyl; where the cyclic moiety of the benzyl and phenyl radicals may in each case be substituted by 1, 2, 3, 4 or 5 substituents selected independently from the group consisting of F, Cl, Br and —CF₃;
or is one of the following radicals 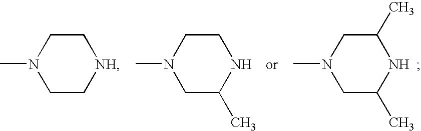
where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃.

47. A method as claimed in claim 35, wherein
R¹ is one of the following radicals 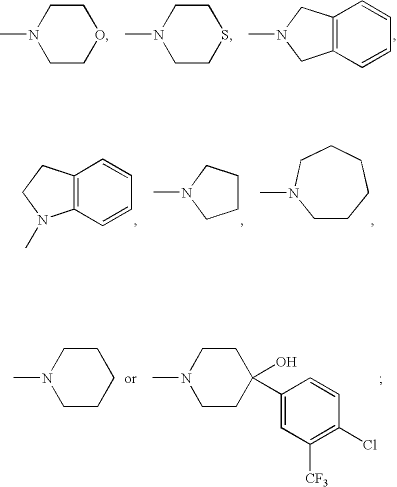
is the following radical 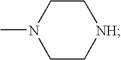
where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may be substituted in each case by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is a radical selected from the group consisting of phenyl and naphthyl, where the radical may in each case optionally be substituted by 1, 2, 3, 4 or 5 substituents selected independently from the group consisting of —OH, —O—CH₃ and —O—C₂H₅;
is an NR³R⁴ group
or is an NR⁷—C(═O)—NR⁸R⁹ group;
R² is a 1,3-dihydroindol-2-onyl or a 1,3-dihydroindol-2-thionyl radical;
is CH—X;
is CH—CH₂—Y, CH—CH(CH₃)—Y or CH—CH(OH)—Y;
or is CH—CH═C(CH₃)-Z, CH—CH═CH-Z, CH—CH═CH—S-Z, CH—C(CH₃)═CH-Z, CH—C(phenyl)=CH-Z, CH—CBr═CH-Z, CH—CCl═CH-Z, CH—CF═CH-Z, CH—CH₂—CH₂-Z, CH—CH₂—CH(CH₃)-Z or CH—CH(CH₃)—CH₂-Z;
R³ is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
or is a radical selected from the group consisting of phenyl, naphthyl, benzyl, (1,3)-benzodioxolyl and (1,4)-benzodioxanyl; where the radical may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R⁴ is a hydrogen radical
or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R⁷ and R⁸ are each a hydrogen radical;
R⁹ is a phenyl radical which may be substituted by 1, 2, 3, 4 or 5 substituents selected independently from the group consisting of F, Cl, Br and —CF₃;
X is a radical selected from the group consisting of phenyl, naphthyl, phenanthrenyl, (1,3)-benzodioxolyl, (1,4)-benzodioxanyl, thiophenyl, furanyl, pyridinyl, pyridazinyl, pyrazinyl, pyrimidinyl, quinolinyl and isoquinolinyl; which may be substituted by 1, 2, 3, 4 or 5 identical or different substituents R¹;
Y is a phenyl or naphthyl radical which may be substituted by 1, 2, 3 4 or 5 identical or different substituents R¹¹;
Z is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl and n-octyl;
or is a phenyl or naphthyl radical which may be substituted by 1, 2, 3, 4 or 5 identical or different substituents R¹²;
R¹⁰ is a radical selected from the group consisting of F, Cl, Br, I, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —O—CF₃, —S—CF₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —N(CH₃)₂, —N(C₂H₅)₂, —NH—CH₃, —NH—C₂H₅, —NH—C(═O)—CH₃, —O—S(═O)₂-phenyl, —O-phenyl, —O-benzyl and phenyl;
is a —C(═O)—NR¹³—(CH₂)ₘ—NR¹⁴—R^{X} group where m is 0, 1, 2 or 3;
or is a —C(═O)—R¹⁶ group;
R¹¹ is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl;
R¹² is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —NO₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl;
R¹³ and R¹⁴ are each a hydrogen radical;
R¹⁵ is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CF₃, —S—CF₃, methyl, ethyl, n-propyl, isopropyl, n-butyl, sec-butyl, isobutyl and tert-butyl; and
R¹⁶ is one of the following radicals 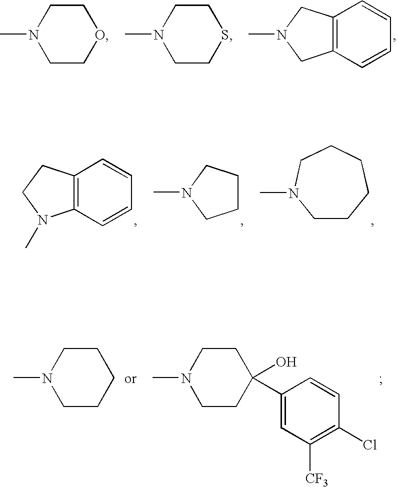
or is one of the following radicals 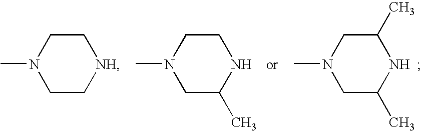
where the hydrogen atom of the —NH group may be replaced by a substituent selected from the group consisting of pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃.

48. A method as claimed in claim 35, selected from the group consisting of:
1 5-[3-(4-methoxyphenyl)but-2-enylidene]-2-morpholin-4-ylthiazol-4-one;
2 2-morpholin-4-yl-5-phenethylidenethiazol-4-one;
3 2-morpholin-4-yl-5-(2-o-tolylethylidene)thiazol-4-one;
4 2-morpholin-4-yl-5-(3-phenylallylidene)thiazol-4-one;
5 2-morpholin-4-yl-5-(3-phenylbutylidene)thiazol-4-one;
6 2-morpholin-4-yl-5-(3-phenylpropylidene)thiazol-4-one;
7 5-[3-(4-fluorophenyl)allylidene]-2-morpholin-4-ylthiazol-4-one;
8 N-[4-(2-morpholin-4-yl-4-oxo-4H-thiazol-5-ylidenemethyl)phenyl]acetamide
9 5-benzo[1,3]dioxol-5-ylmethylene-2-morpholin-4-ylthiazol-4-one;
10 2-morpholin-4-yl-5-(3-m-tolylallylidene)thiazol-4-one;
11 5-(3-iodo-4,5-dimethoxybenzylidene)-2-morpholin-4-ylthiazol-4-one;
12 2-morpholin-4-yl-5-(3-phenylsulfanylallylidene)thiazol-4-one;
13 5-(3-benzyloxy-4-methoxybenzylidene)-2-morpholin-4-ylthiazol-4-one;
14 5-(4-methylbenzylidene)-2-thiomorpholin-4-ylthiazol-4-one;
15 N-[4-(2-morpholin-4-yl-4-oxo-4H-thiazol-5-ylidenemethyl)phenyl]amine;
16 5-(4-butoxybenzylidene)-2-morpholin-4-ylthiazol-1-one;
17 2-morpholin-4-yl-5-(4-phenoxybenzylidene)thiazol-4-one;
18 2-morpholin-4-yl-5-(3-phenoxybenzylidene)thiazol-4-one;
19 2-morpholin-4-yl-5-(3-p-tolylallylidene)thiazol-4-one;
20 5-(3-benzyloxybenzylidene)-2-morpholin-4-ylthiazol-4-one;
21 5-(4-benzyloxybenzylidene)-2-morpholin-4-ylthiazol-4-one;
22 2-thiomorpholin-4-yl-5-(2-trifluoromethylbenzylidene)thiazol-4-one;
23 5-(4-bromobenzylidene)-2-thiomorpholin-4-ylthiazol-4-one;
24 2-thiomorpholin-4-yl-5-(4-trifluoromethoxybenzylidene)thiazol-4-one;
25 5-(3-phenylallylidene)-2-thiomorpholin-4-ylthiazol-4-one;
26 2-morpholin-4-yl-5-octylidenethiazol-4-one;
27 5-(4-benzyloxy-3-methoxybenzylidene)-2-morpholin-4-ylthiazol-4-one;
28 5-(3,4-bis(benzyloxy)benzylidene)-2-morpholin-4-ylthiazol-4-one;
29 5-butylidene-2-morpholin-4-ylthiazol-4-one;
30 2-[4-(3-chloropyridin-2-yl)piperazin-1-yl]-5-(4-methylbenzylidene)thiazol-4-one;
31 2-[4-(3-chloropyridin-2-yl)piperazin-1-yl]-5-(3-phenylallylidene)thiazol-4-one;
32 5-hexylidene-2-morpholin-4-ylthiazol-4-one;
33 2-morpholin-4-yl-5-(3-p-tolyl but-2-enylidene)thiazol-4-one;
34 5-[3-(4-tert-butylphenyl)but-2-enylidene]-2-morpholin-4-ylthiazol-4-one;
35 5-(4-methoxynaphthalen-1-ylmethylene)-2-morpholin-4-ylthiazol-4-one;
36 2-morpholin-4-yl-5-(4-trifluoromethoxybenzylidene)thiazol-4-one;
37 2-[4-(3-methoxyphenyl)piperazin-1-yl]-5-(3-phenylallylidene)thiazol-4-one;
38 5-[3-(4-tert-butylphenyl)allylidene]-2-[4-(3-methoxyphenyl)piperazin-1-yl]-thiazol-4-one;
39 5-(4-hydroxy-3-methoxybenzylidene)-2-thiomorpholin-4-ylthiazol-4-one;
40 5-[3-(4-hydroxy-3-methoxyphenyl)allylidene]-2-thiomorpholin-4-ylthiazol-4-one;
41 5-naphthalen-1-ylmethylene-2-thiomorpholin-4-ylthiazol-4-one;
42 5-benzylidene-2-thiomorpholin-4-ylthiazol-4-one;
43 2-morpholin-4-yl-5-[3-(4-trifluoromethylphenyl)but-2-enylidene]thiazol-4-one;
44 2-morpholin-4-yl-5-[3-(3-trifluoromethylphenyl)but-2-enylidene]thiazol-4-one;
45 5-(3-methylbutylidene)-2-morpholin-4-ylthiazol-4-one;
46 2-[4-(3-chloropyridin-2-yl)piperazin-1-yl]-5-(2-trifluoromethylbenzylidene)thiazol-4-one;
47 5-(4-bromobenzylidene)-2-[4-(3-chloropyridin-2-yl)piperazin-1-yl]-thiazol-4-one;
48 5-benzylidene-2-[4-(3-chloropyridin-2-yl)piperazin-1-yl]thiazol-4-one;
49 2-[4-(3-chloropyridin-2-yl)piperazin-1-yl]-5-naphthalen-2-ylmethylenethiazol-4-one;
50 5-(2,3-diphenylallylidene)-2-morpholin-4-ylthiazol-4-one;
51 2-morpholin-4-yl-5-phenanthren-9-ylmethylenethiazol-4-one;
52 2-morpholin-4-yl-5-quinolin-3-ylmethylenethiazol-4-one;
53 2-(4-ethylpiperazin-1-yl)-5-(4-methylbenzylidene)thiazol-4-one;
54 5-naphthalen-2-ylmethylene-2-thiomorpholin-4-ylthiazol-4-one;
55 2-[4-(4-methoxyphenyl)piperazin-1-yl]-5-(3-phenylallylidene)thiazol-4-one;
56 2-[4-(2-chlorophenyl)piperazin-1-yl]-5-(3-phenylallylidene)thiazol-4-one;
57 5-(3-phenylallylidene)-2-(4-phenylpiperazin-1-yl)thiazol-4-one;
58 5-decylidene-2-morpholin-4-ylthiazol-4-one;
59 5-[3-(4-tert-butylphenyl)allylidene]-2-morpholin-4-ylthiazol-4-one;
60 2-(2-morpholin-4-yl-4-oxo-4H-thiazol-5-ylidenemethyl)benzoic acid;
61 5-(2-bromo-3-phenylallylidene)-2-[4-(3-chloropyridin-2-yl)piperazin-1-yl]thiazol-4-one;
62 5-[3-(4-tert-butylphenyl)allylidene]-2-[4-(3-chloropyridin-2-yl)piperazin-1-yl]-thiazol-4-one;
63 2-(4-benzylpiperazin-1-yl)-5-(4-methylbenzylidene)thiazol-4-one;
64 2-(4-phenylpiperazin-1-yl)-5-quinolin-3-ylmethylenethiazol-4-one;
65 2-[4-(3-chloropyridin-2-yl)piperazin-1-yl]-5-(2-methyl-3-phenylallylidene)thiazol-4-one;
66 2-[4-(3-chloropyridin-2-yl)piperazin-1-yl]-5-naphthalen-1-ylmethylenethiazol-4-one;
67 5-(2-chloro-3-phenylallylidene)-2-[4-(3-chloropyridin-2-yl)piperazin-1-yl]thiazol-4-one;
68 5-(4-hydroxy-3-methoxybenzylidene)-2-(4-phenylpiperazin-1-yl)thiazol-4-one;
69 5-phenanthren-9-ylmethylene-2-(4-phenylpiperazin-1-yl)thiazol-4-one;
70 5-(6-methoxynaphthalen-2-ylmethylene)-2-(4-phenylpiperazin-1-yl)thiazol-4-one;
71 5-naphthalen-1-ylmethylene-2-(4-phenylpiperazin-1-yl)thiazol-4-one;
72 5-(4-bromobenzylidene)-2-(4-phenylpiperazin-1-yl)thiazol-4-one;
73 5-(4-methylbenzylidene)-2-(4-phenylpiperazin-1-yl)thiazol-4-one;
74 2-(4-phenyl piperazin-1-yl)-5-(2-trifluoromethylbenzylidene)thiazol-4-one;
75 3-(4-oxo-2-thiomorpholin-4-yl-4H-thiazol-5-ylidene)-1,3-dihydroindol-2-one;
76 3-(2-morpholin-4-yl-4-oxo-4H-thiazol-5-ylidene)-1,3-dihydroindol-2-one;
77 2-[4-(2-fluorophenyl)piperazin-1-yl]-5-(4-methylbenzylidene)thiazol-4-one;
78 5-(4-tert-butylbenzylidene)-2-[4-(2-fluorophenyl)piperazin-1-yl]thiazol-4-one;
79 2-[4-(2-fluorophenyl)piperazin-1-yl]-5-(3-phenylallylidene)thiazol-4-one;
80 5-[3-(4-tert-butylphenyl)allylidene]-2-[4-(2-fluorophenyl)piperazin-1-yl]thiazol-4-one;
81 2-[4-(2-fluorophenyl)piperazin-1-yl]-5-(4-trifluoromethylbenzylidene)thiazol-4-one;
82 2-[4-(2-fluorophenyl)piperazin-1-yl]-5-(4-trifluoromethoxybenzylidene)thiazol-4-one;
83 5-(2-chloro-3-phenylallylidene)-2-[4-(2-fluorophenyl)piperazin-1-yl]thiazol-4-one;
84 2-[4-(2-fluorophenyl)piperazin-1-yl]-5-naphthalen-1-ylmethylenethiazol-4-one;
85 5-(2-chloro-3-phenylallylidene)-2-thiomorpholin-4-ylthiazol-4-one;
86 3-(4-oxo-2-thiomorpholin-4-yl-4H-thiazol-5-ylidenemethyl)benzoic acid
87 4-(4-oxo-2-thiomorpholin-4-yl-4H-thiazol-5-ylidenemethyl)benzoic acid
88 5-{3-[4-(3-chloropyridin-2-yl)piperazine-1-carbonyl]benzylidene}-2-thiomorpholin-4-ylthiazol-4-one
89 5-{4-[4-(3-chloropyridin-2-yl)piperazine-1-carbonyl]benzylidene}-2-thiomorpholin-4-ylthiazol-4-one;
90 2-[4-(3-chloropyridin-2-yl)piperazin-1-yl]-5-quinolin-3-ylmethylenethiazol-4-one;
91 2-[4-(3-chloropyridin-2-yl)piperazin-1-yl]-5-(4-trifluoromethylbenzylidene)thiazol-4-one;
92 2-(2,3-dihydroindol-1-yl)-5-(4-methylbenzylidene)thiazol-4-one;
93 2-(2,3-dihydroindol-1-yl)-5-(4-trifluoromethylbenzylidene)thiazol-4-one;
94 5-[4-(morpholine-4-carbonyl)benzylidene]-2-thiomorpholin-4-ylthiazol-4-one;
95 2-[4-(3-chloropyridin-2-yl)piperazin-1-yl]-5-(4-hydroxy-3-methoxybenzylidene)thiazol-4-one;
96 1-[4-oxo-5-(4-trifluoromethylbenzylidene)-4,5-dihydrothiazol-2-yl]-3-(2-trifluoromethylphenyl)urea
97 2-(4-benzylpiperazin-1-yl)-5-quinolin-3-ylmethylenethiazol-4-one;
98 2-(4-benzylpiperazin-1-yl)-5-naphthalen-2-ylmethylenethiazol-4-one;
99 2-(4-benzylpiperazin-1-yl)-5-(3-phenoxybenzylidene)thiazol-4-one;
100 2-(4-benzylpiperazin-1-yl)-5-(4-trifluoromethylbenzylidene)thiazol-4-one;
101 1-[5-(4-methylbenzylidene)-4-oxo-4,5-dihydrothiazol-2-yl]-3-(2-trifluoromethylphenyl)urea
102 2-(4-benzylpiperazin-1-yl)-5-naphthalen-1-ylmethylenethiazol-4-one;
103 2-(4-benzylpiperazin-1-yl)-5-(6-methoxynaphthalen-2-ylmethylene)thiazol-4-one;
104 2-(4-benzylpiperazin-1-yl)-5-(4-tert-butylbenzylidene)thiazol-4-one;
105 1-[4-oxo-5-(3-phenylallylidene)-4,5-dihydrothiazol-2-yl]-3-(2-trifluoromethylphenyl)urea
106 1-[4-oxo-5-(4-trifluoromethylbenzylidene)-4,5-dihydrothiazol-2-yl]-3-(4-trifluoromethylphenyl)urea
107 1-[5-(4-methylbenzylidene)-4-oxo-4,5-dihydrothiazol-2-yl]-3-(4-trifluoromethylphenyl)urea
108 5-{3-[4-(6-chloropyridin-2-yl)piperazine-1-carbonyl]benzylidene}-2-thiomorpholin-4-ylthiazol-4-one;
109 2-(4-benzylpiperazin-1-yl)-5-(4-trifluoromethoxybenzylidene)thiazol-4-one;
110 5-{3-[4-(3-chloropyridin-2-yl)-3-methylpiperazine-1-carbonyl]benzylidene}-2-thiomorpholin-4-ylthiazol-4-one;
111 N-[3-(3-chloro-5-trifluoromethylpyridin-2-ylamino)propyl]-3-(4-oxo-2-thiomorpholin-4-yl-4H-thiazol-5-ylidenemethyl)benzamide
112 3-(4-oxo-2-thiomorpholin-4-yl-4H-thiazol-5-ylidenemethyl)benzoic acid N′-(2-chloro-4-trifluoromethylphenyl)hydrazide
113 5-{3-[4-(3-chloro-5-trifluoromethylpyridin-2-yl)piperazine-1-carbonyl]-benzylidene}-2-thiomorpholin-4-ylthiazol-4-one;
114 3-(4-oxo-2-thiomorpholin-4-yl-4H-thiazol-5-ylidenemethyl)benzoic acid N′-(3-chloro-5-trifluoromethylpyridin-2-yl)hydrazide
115 5-{3-[4-(4-chloro-[1,2,5]thiadiazol-3-yl)piperazine-1-carbonyl]benzylidene}-2-thiomorpholin-4-ylthiazol-4-one;
116 3-(4-oxo-2-thiomorpholin-4-yl-4H-thiazol-5-ylidenemethyl)benzoic acid N′-(2-chloro-5-trifluoromethylphenyl)hydrazide
117 2-[4-(4-chloro-[1,2,5]thiadiazol-3-yl)piperazin-1-yl]-5-(4-methylbenzylidene)thiazol-4-one;
118 2-[4-(4-chloro-[1,2,5]thiadiazol-3-yl)piperazin-1-yl]-5-(4-trifluoromethylbenzylidene)thiazol-4-one;
119 2-[4-(4-chloro-[1,2,5]thiadiazol-3-yl)piperazin-1-yl]-5-(4-trifluoromethoxybenzylidene)thiazol-4-one
120 5-(4-tert-butylbenzylidene)-2-[4-(4-chloro-[1,2,5]thiadiazol-3-yl)piperazin-1-yl]thiazol-4-one;
121 2-[4-(4-chloro-[1,2,5]thiadiazol-3-yl)piperazin-1-yl]-5-(3-phenyl-allylidene)thiazol-4-one;
122 5-[3-(4-tert-butylphenyl)allylidene]-2-[4-(4-chloro-[1,2,5]thiadiazol-3-yl)-piperazin-1-yl]thiazol-4-one;
123 2-[4-(4-chloro-[1,2,5]thiadiazol-3-yl)piperazin-1-yl]-5-naphthalen-1-ylmethylenethiazol-4-one;
124 2-[4-(4-chloro-[1,2,5]thiadiazol-3-yl)piperazin-1-yl]-5-(3-phenoxybenzylidene)thiazol-4-one;
125 2-[4-(4-chloro-[1,2,5]thiadiazol-3-yl)piperazin-1-yl]-5-(4-phenoxybenzylidene)thiazol-4-one;
126 5-(3-benzyloxybenzylidene)-2-[4-(4-chloro-[1,2,5]thiadiazol-3-yl)piperazin-1-yl]thiazol-4-one;
127 2-[4-(4-chloro-[1,2,5]thiadiazol-3-yl)piperazin-1-yl]-5-(4-hydroxy-3-methoxybenzylidene)thiazol-4-one;
128 2-[4-(4-chloro-[1,2,5]thiadiazol-3-yl)piperazin-1-yl]-5-furan-2-yl-methylenethiazol-4-one;
129 2-[4-(4-chloro-[1,2,5]thiadiazol-3-yl)piperazin-1-yl]-5-naphthalen-2-yl-methylenethiazol-4-one;
130 3-[2-(4-benzylpiperazin-1-yl)-4-oxo-4H-thiazol-5-ylidenemethyl]benzoic acid
131 5-naphthalen-1-ylmethylene-2-piperazin-1-ylthiazol-4-one;
132 2-piperazin-1-yl-5-(4-trifluoromethylbenzylidene)thiazol-4-one;
133 5-(4-methylbenzylidene)-2-piperazin-1-ylthiazol-4-one;
134 5-(4-hydroxy-3-methoxybenzylidene)-2-piperazin-1-ylthiazol-4-one;
135 5-(4-isopropylbenzylidene)-2-morpholin-4-ylthiazol-4-one;
136 5-(4-isopropylbenzylidene)-2-thiomorpholin-4-ylthiazol-4-one;
137 2-[4-(2-fluorophenyl)piperazin-1-yl]-5-(4-isopropylbenzylidene)thiazol-4-one;
138 5-(4-pentafluorosulfanyl)benzylidene)-2-thiomorpholin-4-ylthiazol-4-one;
139 2-[4-(3-chloropyridin-2-yl)piperazin-1-yl]-5-(4-isopropylbenzylidene)thiazol-4-one;
140 2-(4-benzylpiperazin-1-yl)-5-(4-butylbenzylidene)thiazol-4-one;
141 2-(4-benzylpiperazin-1-yl)-5-(4-pentylbenzylidene)thiazol-4-one;
142 2-(4-benzylpiperazin-1-yl)-5-(4-octylbenzylidene)thiazol-4-one;
143 2-[4-(6-chloropyridin-2-yl)piperazin-1-yl]-5-(4-methylbenzylidene)thiazol-4-one;
144 5-(4-tert-butylbenzylidene)-2-[4-(6-chloropyridin-2-yl)piperazin-1-yl]thiazol-4-one;
145 2-[4-(6-chloropyridin-2-yl)piperazin-1-yl]-5-(4-trifluoromethylbenzylidene)thiazol-4-one;
146 2-[4-(6-chloropyridin-2-yl)piperazin-1-yl]-5-(3-phenylallylidene)thiazol-4-one;
147 5-[3-(4-tert-butylphenyl)allylidene]-2-[4-(6-chloropyridin-2-yl)piperazin-1-yl]thiazol-4-one;
148 2-[4-(6-chloropyridin-2-yl)piperazin-1-yl]-5-(4-hydroxy-3-methoxybenzylidene)thiazol-4-one;
149 2-[4-(6-chloropyridin-2-yl)piperazin-1-yl]-5-(4-isopropylbenzylidene)thiazol-4-one;
150 2-[4-(6-chloropyridin-2-yl)piperazin-1-yl]-5-(4-trifluoromethoxybenzylidene)thiazol-4-one;
151 2-[4-(6-chloropyridin-2-yl)piperazin-1-yl]-5-naphthalen-2-ylmethylenethiazol-4-one
152 5-(4-methylbenzylidene)-2-[4-(6-methylpyridazin-3-yl)piperazin-1-yl]thiazol-4-one;
153 5-(4-isopropylbenzylidene)-2-[4-(6-methylpyridazin-3-yl)piperazin-1-yl]thiazol-4-one;
154 5-(4-tert-butylbenzylidene)-2-[4-(6-methylpyridazin-3-yl)piperazin-1-yl]thiazol-4-one;
155 5-[3-(4-tert-butylphenyl)allylidene]-2-[4-(6-methylpyridazin-3-yl)piperazin-1-yl]thiazol-4-one;
156 2-[4-(6-methylpyridazin-3-yl)piperazin-1-yl]-5-(4-trifluoromethoxybenzylidene)thiazol-4-one;
157 5-(4-hydroxy-3-methoxybenzylidene)-2-[4-(6-methylpyridazin-3-yl)piperazin-1-yl]thiazol-4-one;
158 2-(4-benzylpiperazin-1-yl)-5-(4-pentafluorosulfanylbenzylidene)thiazol-4-one;
159 2-(4-benzylpiperazin-1-yl)-5-{3-[4-(4-chloro-[1,2,5]thiadiazol-3-yl)piperazine-1-carbonyl]benzylidene}thiazol-4-one;
160 2-[4-(6-methylpyridazin-3-yl)piperazin-1-yl]-5-(4-trifluoromethylbenzylidene)thiazol-4-one;
161 2-[4-(6-methylpyridazin-3-yl)piperazin-1-yl]-5-(3-phenylallylidene)thiazol-4-one;
162 2-[4-(6-methylpyridazin-3-yl)piperazin-1-yl]-5-naphthalen-2-yl-methylenethiazol-4-one;
163 2-[4-(6-methylpyridazin-3-yl)piperazin-1-yl]-5-naphthalen-1-yl-methylenethiazol-4-one;
164 2-(4-benzylpiperazin-1-yl)-5-(4-isopropylbenzylidene)thiazol-4-one;
165 3-{2-[4-(4-chloro-[1,2,5]thiadiazol-3-yl)piperazin-1-yl]-4-oxo-4H-thiazol-5-ylidenemethyl}benzoic acid;
166 5-{3-[4-(4-chloro-3-trifluoromethylphenyl)-4-hydroxypiperidine-1-carbonyl]benzylidene}-2-thiomorpholin-4-ylthiazol-4-one;
167 5-{3-[4-(4-chloro-[1,2,5]thiadiazol-3-yl)piperazine-1-carbonyl]benzylidene}-2-[4-(4-chloro-[1,2,5]thiadiazol-3-yl)piperazin-1-yl]thiazol-4-one;
168 5-{3-[4-(3-chloropyridin-2-yl)piperazine-1-carbonyl]benzylidene}-2-[4-(4-chloro-[1,2,5]thiadiazol-3-yl)piperazin-1-yl]thiazol-4-one;
169 5-[3-(4-benzylpiperazine-1-carbonyl)benzylidene]-2-[4-(4-chloro-[1,2,5]thiadiazol-3-yl)piperazin-1-yl]thiazol-4-one;
170 2-[4-(4-chloro-[1,2,5]thiadiazol-3-yl)piperazin-1-yl]-5-{3-[4-(6-methoxy-pyridin-2-yl)piperazine-1-carbonyl]benzylidene}thiazol-4-one;
171 2-[4-(4-chloro-3-trifluoromethylphenyl)-4-hydroxypiperidin-1-yl]-5-(4-methylbenzylidene)thiazol-4-one;
172 2-[4-(4-chloro-3-trifluoromethylphenyl)-4-hydroxypiperidin-1-yl]-5-(4-hydroxy-3-methoxybenzylidene)thiazol-4-one;
173 2-[4-(4-chloro-3-trifluoromethylphenyl)-4-hydroxypiperidin-1-yl]-5-naphthalen-1-ylmethylenethiazol-4-one;
174 2-[4-(4-chloro-3-trifluoromethylphenyl)-4-hydroxypiperidin-1-yl]-5-(3-methoxybenzylidene)thiazol-4-one;
175 5-benzylidene-2-morpholin-4-ylthiazol-4-one;
176 5-(4-methyl benzylidene)-2-morpholin-4-ylthiazol-4-one;
177 2-diethylamino-5-(4-methylbenzylidene)thiazol-4-one;
178 5-(4-chlorobenzylidene)-2-diethylaminothiazol-4-one;
179 5-(4-isopropylbenzylidene)-2-morpholin-4-ylthiazol-4-one;
180 5-benzylidene-2-diethylaminothiazol-4-one;
181 5-(4-chlorobenzylidene)-2-morpholin-4-ylthiazol-4-one;
182 5-(4-chlorobenzylidene)-2-piperidin-1-ylthiazol-4-one
183 5-(4-chlorobenzylidene)-2-pyrrolidin-1-ylthiazol-4-one;
184 5-benzylidene-2-pyrrolidin-1-ylthiazol-4-one;
185 5-benzylidene-2-pyrrolidin-1-ylthiazol-4-one;
186 5-biphenyl-4-ylmethylene-2-piperidin-1-ylthiazol-4-one;
187 2-azepan-1-yl-5-biphenyl-4-ylmethylenethiazol-4-one;
188 5-biphenyl-4-ylmethylene-2-pyrrolidin-1-ylthiazol-4-one;
189 2-morpholin-4-yl-5-(3-phenylallylidene)thiazol-4-one;
190 5-(3-phenylallylidene)-2-thiomorpholin-4-ylthiazol-4-one;
191 5-(3-phenylallylidene)-2-piperidin-1-ylthiazol-4-one;
192 2-azepan-1-yl-5-(3-phenylallylidene)thiazol-4-one;
193 5-(3-phenylallylidene)-2-pyrrolidin-1-ylthiazol-4-one;
194 5-biphenyl-4-ylmethylene-2-morpholin-4-ylthiazol-4-one;
195 2-thiomorpholin-4-yl-5-(3-trifluoromethylbenzylidene)thiazol-4-one;
196 5-(4-chlorobenzylidene)-2-thiomorpholin-4-ylthiazol-4-one;
197 5-(4-tert-butylbenzylidene)-2-morpholin-4-ylthiazol-4-one;
198 5-(4-tert-butylbenzylidene)-2-thiomorpholin-4-ylthiazol-4-one;
199 5-(4-tert-butylbenzylidene)-2-pyrrolidin-1-ylthiazol-4-one;
200 2-azepan-1-yl-5-(4-tert-butylbenzylidene)thiazol-4-one;
201 5-(4-tert-butylbenzylidene)-2-piperidin-1-ylthiazol-4-one;
202 2-thiomorpholin-4-yl-5-(4-trifluoromethylbenzylidene)thiazol-4-one;
203 5-(4-tert-butylbenzylidene)-2-diethylaminothiazol-4-one;
204 2-morpholin-4-yl-5-(4-trifluoromethylbenzylidene)thiazol-4-one;
205 2-morpholin-4-yl-5-(3-trifluoromethylbenzylidene)thiazol-4-one;
206 2-morpholin-4-yl-5-(3-trifluoromethylbenzylidene)thiazol-4-one;
207 5-biphenyl-4-ylmethylene-2-diethylaminothiazol-4-one;
208 2-morpholin-4-yl-5-thiophen-2-ylmethylenethiazol-4-one;
209 2-morpholin-4-yl-5-naphthalen-1-ylmethylenethiazol-4-one;
210 2-morpholin-4-yl-5-pyridin-2-ylmethylenethiazol-4-one;
211 2-morpholin-4-yl-5-(4-trifluoromethylbenzylidene)thiazol-4-one;
212 2-morpholin-4-yl-5-pyridin-3-ylmethylenethiazol-4-one;
213 2-morpholin-4-yl-5-pyridin-4-ylmethylenethiazol-4-one;
214 2-diethylamino-5-(3-trifluoromethylbenzylidene)thiazol-4-one;
215 5-(4-methylbenzylidene)-2-pyrrolidin-1-ylthiazol-4-one;
216 5-(4-tert-butylbenzylidene)-2-(2-methoxyethylamino)thiazol-4-one;
217 2-morpholin-4-yl-5-(4-octylbenzylidene)thiazol-4-one;
218 5-(4-methylbenzylidene)-2-morpholin-4-ylthiazol-4-one;
219 5-(3,4-dimethoxybenzylidene)-2-morpholin-4-ylthiazol-4-one;
220 5-(2-hydroxybenzylidene)-2-morpholin-4-ylthiazol-4-one;
221 5-(2-hydroxy-3-methoxybenzylidene)-2-morpholin-4-ylthiazol-4-one;
222 5-(4-tert-butylbenzylidene)-2-morpholin-4-ylthiazol-4-one;
223 5-(4-diethylaminobenzylidene)-2-morpholin-4-ylthiazol-4-one;
224 5-(4-hydroxy-3-methoxybenzylidene)-2-morpholin-4-ylthiazol-4-one;
225 4-(2-morpholin-4-yl-4-oxo-4H-thiazol-5-ylidenemethyl)benzoic acid
226 2-morpholin-4-yl-5-(2,3,4-trimethoxybenzylidene)thiazol-4-one;
227 2-morpholin-4-yl-5-(3,4,5-trimethoxybenzylidene)thiazol-4-one;
228 5-(4-methoxybenzylidene)-2-morpholin-4-ylthiazol-4-one;
229 5-(4-hydroxybenzylidene)-2-morpholin-4-ylthiazol-4-one;
230 5-(3-ethoxy-4-hydroxybenzylidene)-2-morpholin-4-ylthiazol-4-one;
231 5-(3-hydroxybenzylidene)-2-morpholin-4-ylthiazol-4-one;
232 5-(4-bromobenzylidene)-2-morpholin-4-ylthiazol-4-one;
233 2-morpholin-4-yl-5-(4-vinyloxybenzylidene)thiazol-4-one
234 benzenesulfonic acid 4-(2-morpholin-4-yl-4-oxo-4H-thiazol-5-ylidenemethyl)phenyl ester;
235 5-(4-dimethylaminobenzylidene)-2-morpholin-4-ylthiazol-4-one;
236 4-(2-morpholin-4-yl-4-oxo-4H-thiazol-5-ylidenemethyl)benzoic acid methyl ester;
237 5-(3-hydroxy-4-methoxybenzylidene)-2-morpholin-4-ylthiazol-4-one;
238 5-[4-(3,3-dimethyl butoxy)-3-methoxybenzylidene]-2-morpholin-4-ylthiazol-4-one;
239 5-(2-methoxybenzylidene)-2-morpholin-4-ylthiazol-4-one;
240 5-(4-hydroxybenzylidene)-2-morpholin-4-ylthiazol-4-one;
241 2-[(benzo[1,3]dioxol-5-ylmethyl)amino]-5-[3-(4-tert-butylphenyl)allylidene]thiazol-4-one;
242 2-[(benzo[1,3]dioxol-5-ylmethyl)amino]-5-(4-trifluoromethylbenzylidene)thiazol-4-one;
243 2-[(benzo[1,3]dioxol-5-ylmethyl)amino]-5-(4-trifluoromethoxybenzylidene)thiazol-4-one;
244 2-[(benzo[1,3]dioxol-5-ylmethyl)amino]-5-naphthalen-1-ylmethylenethiazol-4-one;
245 2-[(benzo[1,3]dioxol-5-ylmethyl)amino]-5-(2-chloro-3-phenylallylidene)thiazol-4-one;
246 2-(4-methylbenzylamino)-5-naphthalen-1-ylmethylenethiazol-4-one;
247 2-(4-methylbenzylamino)-5-(3-phenoxybenzylidene)thiazol-4-one;
248 2-(4-methylbenzylamino)-5-(4-phenoxybenzylidene)thiazol-4-one;
249 5-(2-chloro-3-phenylallylidene)-2-(4-methylbenzylamino)thiazol-4-one;
250 2-(4-methyl benzylamino)-5-naphthalen-2-ylmethylenethiazol-4-one;
251 2-(3-methoxybenzylamino)-5-(4-methylbenzylidene)thiazol-4-one;
252 5-(4-tert-butylbenzylidene)-2-(3-methoxybenzylamino)thiazol-4-one;
253 2-(3-methoxybenzylamino)-5-(4-trifluoromethylbenzylidene)thiazol-4-one;
254 2-(3-methoxybenzylamino)-5-(4-trifluoromethoxybenzylidene)thiazol-4-one;
255 2-(3-methoxybenzylamino)-5-(3-phenylallylidene)thiazol-4-one;
256 5-naphthalen-1-ylmethylene-2-(4-trifluoromethylbenzylamino)thiazol-4-one;
257 5-(3-phenoxybenzylidene)-2-(4-trifluoromethylbenzylamino)thiazol-4-one;
258 5-(4-phenoxybenzylidene)-2-(4-trifluoromethylbenzylamino)thiazol-4-one;
259 5-(2-chloro-3-phenylallylidene)-2-(4-trifluoromethylbenzylamino)thiazol-4-one;
260 5-(3-benzyloxybenzylidene)-2-(4-trifluoromethylbenzylamino)thiazol-4-one;
261 5-naphthalen-2-ylmethylene-2-(4-trifluoromethylbenzylamino)thiazol-4-one;
262 2-(4-hydroxy-3-methoxyphenyl)-5-(4-methylbenzylidene)thiazol-4-one; and
263 2-(4-tert-butylphenylamino)-5-(4-methylbenzylidene)thiazol-4-one;
or a pharmaceutically acceptable salt or solvate thereof.

49. A 2,5-disubstituted thiazol-4-one compound corresponding to formula Ia 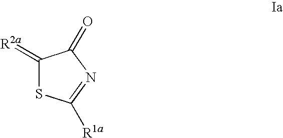
wherein
R^{1a} is an unsubstituted or at least monosubstituted, unsaturated or saturated cycloaliphatic radical which optionally has at least one heteroatom as a ring member and may be fused to an unsubstituted or at least monosubstituted mono- or polycyclic ring system;
is an NR^{3a}R^{4a} group;
is an NR^{5a}—C(═O)—R^{6a} group;
or is an NR^{7a}—C(═O)—NR^{8a}R^{9a} group;
R^{2a} is CH—CH═C(CH₃)—W^{a}-Z^{a}, CH—CH═CH_W^{a}-Z^{a}, CH—C(CH₃)═CH—W^{a}-Z^{a}, CH—C(phenyl)═CH—W^{a}-Z^{a}, CH—CBr═CH_W^{a}-Z^{a}, CH—CCl═CH—W^{a}-Z^{a}, CH—CF═CH—W^{a}-Z^{a}, CH—C(OH)═CH—W^{a}-Z^{a}, CH—CH₂—CH₂—W^{a}-Z^{a}, CH—CH₂—CH(CH₃)_W^{a}-Z^{a} or CH—CH(CH₃)—CH₂—W^{a}-Z^{a};
where W^{a} may in each case be absent or is in each case a radical selected from the group consisting of O, S, N(H), N(CH₃), N(C₂H₅) and N[CH(CH₃)₂];
R^{3a} is a linear or branched, saturated or unsaturated, unsubstituted or at least monosubstituted aliphatic radical optionally having a heteroatom as a chain member; is an unsubstituted or at least monosubstituted, unsaturated or saturated cycloaliphatic radical which optionally has at least one heteroatom as a ring member and may be fused to an unsubstituted or at least monosubstituted mono- or polycyclic ring system; or is an unsubstituted or at least monosubstituted aryl or heteroaryl radical which may be fused to an unsubstituted or at least monosubstituted mono- or polycyclic ring system and/or may be bonded via a linear or branched, unsubstituted or at least monosubstituted alkylene, alkenylene or alkynylene group;
R^{1a} is a hydrogen radical
or is a linear or branched, saturated or unsaturated, unsubstituted or at least monosubstituted aliphatic radical optionally having at least one heteroatom as a chain member;
R^{5a}, R^{7a} and R^{9a} are each independently
a hydrogen radical
or a linear or branched, saturated or unsaturated, unsubstituted or at least monosubstituted aliphatic radical;
R^{6a} and R^{8a} are each independently
an unsubstituted or at least monosubstituted aryl or heteroaryl radical which may be fused to an unsubstituted or at least monosubstituted mono- or polycyclic ring system;
Z^{a} is a linear or branched, saturated or unsaturated, unsubstituted or at least monosubstituted aliphatic radical;
is an aryl radical which may be fused to an unsubstituted or at least monosubstituted mono- or polycyclic ring system and/or may be substituted by identical or different substituents R^{12a};
or is an heteroaryl radical which may be fused to an unsubstituted or at least monosubstituted mono- or polycyclic ring system and/or may be substituted by identical or different substituents R^{12a};
R^{12a} is a radical selected from the group consisting of F, Cl, Br, I, —CN, —CF₃, —SF₅, —OH, —O—C₁₋₁₀-alkyl, —O—C₂₋₁₀-alkenyl, —NH₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted;
is a C(═O)—NR^{13a}—(CH₂)ₘ—NR^{14a}—R^{15a} group, where m is 0, 1, 2, 3, 4 or 5;
or is a —C(═O)—R^{16a} group;
R^{13a} and R^{14a} are each independently
a hydrogen radical
or a linear or branched, saturated or unsaturated, unsubstituted or at least monosubstituted aliphatic radical;
R^{15a} is an unsubstituted or at least monosubstituted aryl or heteroaryl radical which may be fused to an unsubstituted or at least monosubstituted mono- or polycyclic ring system; and
R^{6a} is an unsubstituted or at least monosubstituted, unsaturated or saturated cycloaliphatic radical which optionally has at least one heteroatom as a ring member and may be fused to an unsubstituted or at least monosubstituted mono- or polycyclic ring system.

50. A compound as claimed in claim 49, wherein said compound is in the form of an isolated or purified stereoisomer.

51. A compound as claimed in claim 49, wherein said compound is in the form of a racemic mixture.

52. A compound as claimed in claim 49, wherein
R^{1a} is an unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
is an NR^{3a}R^{4a} group;
is an NR^{5a}—C(═O)—R^{6a} group
or is an NR^{7a}—C(═O)—NR^{8a}R^{9a} group;
R^{2a} is CH—CH═C(CH₃)_W^{a}-Z^{a}, CH—CH═CH_W^{a}-Z^{a}, CH—C(CH₃)═CH_W^{a}-Z^{a}, CH—C(phenyl)=CH_W^{a}-Z^{a}, CH—CBr═CH—W^{a}-Z^{a}, CH—CCl═CH—W^{a}-Z^{a}, CH—CF═CH—W^{a}-Z^{a}, CH—C(OH)═CH—W^{a}-Z^{a}, CH—CH₂—CH₂—W^{a}-Z^{a}, CH—CH₂—CH(CH₃)_W^{a}-Z^{a} or CH—CH(CH₃)—CH₂—W^{a}-Z^{a};
where W^{a} may in each case be absent or is in each case a radical selected from the group consisting of O, S, N(H), N(CH₃), N(C₂H₅) and N[CH(CH₃)₂];
R^{3a} is a linear or branched, saturated or unsaturated, optionally substituted C₁₋₁₀ aliphatic radical optionally having 1, 2 or 3 heteroatoms selected independently from the group consisting of oxygen, nitrogen (NH) and sulfur;
is an unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
or is an optionally substituted 5- to 14-membered aryl or heteroaryl radical which may be fused to a saturated or unsaturated, optionally substituted mono- or polycyclic ring system and/or be bonded via a C₁₋₅-alkylene group;
R^{4a} is a hydrogen radical
or is a linear or branched, saturated or unsaturated, optionally substituted C₁₋₁₀ aliphatic radical optionally having 1, 2 or 3 heteroatoms selected independently from the group consisting of oxygen, nitrogen (NH) and sulfur;
R^{5a}, R^{7a} and R^{8a} are each independently
a hydrogen radical
or a linear or branched, saturated or unsaturated, optionally substituted C₁₋₁₀ aliphatic radical;
R^{6a} and R^{9a} are each independently
an optionally substituted 5- to 14-membered aryl or heteroaryl radical which may be fused to a saturated or unsaturated, optionally substituted mono- or polycyclic ring system;
Z^{a} is a linear or branched, saturated or unsaturated, optionally substituted C₁₋₂₀ aliphatic radical;
is a substituted 6- or 10-membered aryl radical which is fused to a saturated or unsaturated, optionally substituted mono- or polycyclic ring system and/or is substituted by 1, 2, 3, 4 or 5 identical or different substituents R^{12a};
or is an optionally substituted 5- to 14-membered heteroaryl radical which is fused to a saturated or unsaturated, substituted mono- or polycyclic ring system and/or may optionally be substituted by 1, 2, 3, 4 or 5 identical or different substituents R^{12a};
R^{12a} is a radical selected from the group consisting of F, Cl, Br, I, —CN, —CF₃, —SF₅, —OH, —O—C₁₋₁₀-alkyl, —O—C₂₋₁₀-alkenyl, —NH₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —O—S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
a —C(═O)—NR¹³—(CH₂)ₘ—NR¹⁴—R¹⁵ group where m is 0, 1, 2, 3, 4 or 5;
or a —C(═O)—R^{16a} group;
R^{13a} and R^{14a} are each independently
a hydrogen radical
or a linear or branched, saturated or unsaturated, optionally substituted C₁₋₁₀ aliphatic radical;
R^{15a} is a 5- to 14-membered aryl or heteroaryl radical which may be fused to a saturated or unsaturated, optionally substituted mono- or polycyclic ring system; and
R^{16a} is an unsaturated or saturated, optionally substituted 3-, 4-, 5-, 6-, 7-, 8- or 9-membered cycloaliphatic radical which may be fused to a saturated or unsaturated, optionally substituted mono- or polycyclic ring system;
wherein
the aforementioned cycloaliphatic radicals may each optionally be substituted by 1, 2, 3, 4 or 5 substituents selected independently from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH-phenyl, —NH-pyridinyl, —N(C₁₋₅-alkyl)-phenyl, —N(C₁₋₅-alkyl)-pyridinyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, [1,2,5]-thiadiazolyl, pyridazinyl, —(CH₂)— benzo[b]furanyl, —O-phenyl, —O-benzyl, phenyl and benzyl, where the cyclic moiety of the —NH-phenyl, —NH-pyridinyl, —N(C₁₋₅-alkyl)-phenyl, —N(C₁₋₅-alkyl)-pyridinyl, pyridinyl, cyclopentyl, [1,2,5]-thiadiazolyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
and the aforementioned cycloaliphatic radicals may each optionally have 1, 2, 3, 4 or 5 heteroatom(s) selected independently from the group consisting of oxygen, nitrogen and sulfur as ring member(s);
the rings of the aforementioned mono- or polycyclic ring systems may each optionally be substituted by 1, 2, 3, 4 or 5 substituents selected independently from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl, where the cyclic moiety of the pyridinyl, cyclopentyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
and the rings of the aforementioned mono- or polycyclic ring systems are each 5-, 6- or 7-membered and may each optionally have 1, 2, 3, 4 or 5 heteroatom(s) as ring member(s) which are selected independently from the group consisting of oxygen, nitrogen and sulfur; the aforementioned C₁₋₁₀ aliphatic radicals or C₁₋₂₀ aliphatic radicals may each optionally be substituted by 1, 2, 3, 4, 5, 6, 7, 8 or 9 substituents selected independently from the group consisting of F, Cl, Br, I, —CN, —NO₂, —OH, —SH and —NH₂;
the aforementioned aryl or heteroaryl radicals may each optionally be substituted by 1, 2, 3, 4 or 5 substituents selected independently from the group consisting of F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₁₀-alkyl, —C(═O)—OH, —C(═O)—O—C₁₋₅-alkyl, —O—C(═O)—C₁₋₅-alkyl, —NH—C₁₋₅-alkyl, —N(C₁₋₅-alkyl)₂, —NH—C(═O)—O—C₁₋₅-alkyl, —NH—C(═O)—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —S(═O)₂—C₁₋₅-alkyl, —S(═O)₂-phenyl, —NH—S(═O)₂—C₁₋₅-alkyl, —S(═O)₂—NH—C₁₋₅-alkyl, cyclohexyl, cyclopentyl, pyridinyl, pyridazinyl, —(CH₂)-benzo[b]furanyl, —O-phenyl, —O-benzyl, phenyl and benzyl, where the cyclic moiety of the pyridinyl, cyclopentyl, cyclohexyl, pyridazinyl, —S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
and the aforementioned heteroaryl radicals may each optionally have 1, 2, 3, 4 or 5 heteroatom(s) selected independently from the group consisting of oxygen, nitrogen and sulfur as ring member(s);
and the aforementioned C₁₋₅-alkylene groups may each optionally be substituted by 1, 2, 3, 4 or 5 substituents selected independently from the group consisting of F, Cl, Br, —OH, —SH, —NH₂, —CN and NO₂.

53. A compound as claimed in claim 49, wherein
R^{1a} is a radical selected from the group consisting of cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, thiomorpholinyl, azepanyl, (2,3)-dihydro-1H-isoindolyl, (2,3)-dihydroindolyl, (2,3,4,9)-tetrahydro-1H-□-carbolinyl, (2,3,4,9)-tetrahydro-1H-pyrido[2,3-b]indolyl, [1,2,3,4]-tetrahydronaphthyl, [1,2,3,4]-tetrahydroquinolinyl, [1,2,3,4]-tetrahydroisoquinolinyl, [1,2,3,4]-tetrahydroquinazolinyl and [3,4]-dihydro-2H-1,4-benzoxazinyl; where the radical may in each case optionally be substituted by 1, 2, 3, 4 or 5 substituents selected independently from the group consisting of oxo (═O), thioxo (═S), F, Cl, Br, I, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl, —NH—C(═O)—O—CH₃, —NH—C(═O)—O—C₂H₅, —NH—C(═O)—O—C(CH₃)₃, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl and phenyl; where the cyclic moiety of the —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl, pyridinyl, pyridazinyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl; is a piperazinyl radical which may be substituted by 1, 2 or 3 substituents selected independently from the group consisting of methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —C(═O)—C₁₋₅-alkyl, —C(═O)—NH₂, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
is an NR^{3a}R^{4a} group;
is an NR^{5a}—C(═O)—R^{6a} group
or is an NR^{7a}—C(═O)—NR^{8a}R^{9a} group.

54. A compound as claimed in claim 49, wherein
R^{2a} is CH—CH═C(CH₃)-Z^{a}, CH—CH═CH-Z^{a}, CH—CH═CH—S-Z^{a}, CH—CH═CH—O-Z^{a}, CH—CH═CH—N(CH₃)-Z^{a}, CH—C(CH₃)═CH-Z^{a}, CH—C(phenyl)=CH-Z^{a}, CH—CBr═CH-Z^{a}, CH—CCl═CH-Z^{a}, CH—CF═CH-Z^{a}, CH—C(OH)═CH-Z^{a}, CH—CH₂—CH₂-Z^{a}, CH—CH₂—CH(CH₃)-Z^{a} or CH—CH(CH₃)—CH₂-Z^{a}.

55. A compound as claimed in claim 49, wherein
R^{3a} is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂;
is a radical selected from the group consisting of cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, thiomorpholinyl and azepanyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl; where the cyclic moiety of the —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl and —N[CH(CH₃)₂]-pyridinyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, I, —SF₅, —OH, —O—CH₃, —O—C₂H₅, —NH₂, —NO₂, —CF₃, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—CH(CH₃)₂ and —NH—C(CH₃)₃.

56. A compound as claimed in claim 49, wherein
R^{4a} is a hydrogen radical
or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂.

57. A compound as claimed in claim 49, wherein
R^{5a}, R^{7a} and R^{8a} are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl; where the radical may in each case optionally be substituted by 1, 2, 3, 4 or 5 substituents selected independently from the group consisting of F, Cl, Br, I, —CN, —NO₂, —OH, —SH and —NH₂.

58. A compound as claimed in claim 49, wherein
R^{6a} and R^{9a} are each independently
a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃.

59. A compound as claimed in claim 49, wherein
Z^{a} is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl, where the radical may in each case optionally be substituted by 1, 2, 3, 4 or 5 substituents selected independently from the group consisting of F, Cl, Br, I, CN, —NO₂, —OH, —SH and —NH₂;
is a radical selected from the group consisting of phenyl, naphthyl, phenanthrenyl, (1,3)-benzodioxolyl and (1,4)-benzodioxanyl, which is optionally substituted by 1, 2, 3, 4 or 5 identical or different substituents R^{12a};
or is a radical selected from the group consisting of thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R^{12a}.

60. A compound as claimed in claim 49, wherein
R^{12a} a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl, n-octyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —NH—C(═O)—CH₃, —NH—C(═O)—C₂H₅, —NH—C(═O)—C(CH₃)₃, —C(═O)—H, —C(═O)—CH₃, —C(═O)—C₂H₅, —C(═O)—C(CH₃)₃, —C(═O)—NH₂, —C(═O)—NH—CH₃, —C(═O)—NH—C₂H₅, —C(═O)—N—(CH₃)₂, —C(═O)—N—(C₂H₅)₂, —S(═O)₂—CH₃, —S(═O)₂—C₂H₅, —NH—S(═O)₂—CH₃, —NH—S(═O)₂—C₂H₅, —S(═O)₂—NH—CH₃, —S(═O)₂-phenyl, —O—S(═O)₂-phenyl, cyclohexyl, cyclopentyl, pyridinyl, pyridazinyl, —(CH₂)-benzo[b]furanyl, —O-phenyl, —O-benzyl, phenyl and benzyl; where the cyclic moiety of the pyridinyl, cyclopentyl, cyclohexyl, pyridazinyl, —S(═O)₂-phenyl, —O—S(═O)₂-phenyl, —O-phenyl, —O-benzyl, phenyl, —(CH₂)-benzo[b]furanyl and benzyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl;
a —C(═O)—NR^{13a}—(CH₂)ₘ—NR^{14a}—R^{15a} group where m is 0, 1, 2 or 3;
or a —C(═O)—R^{16a} group;
wherein
R^{13a} and R^{14a} are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{15a} is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —OH, —O—CH₃, —O—C₂H₅, —NH₂, —NO₂, —O—CF₃, —S—CF₃, —SH, —S—CH₃, —S—C₂H₅, methyl, ethyl, n-propyl, isopropyl, n-butyl, sec-butyl, isobutyl, tert-butyl, —C(═O)—OH, —C(═O)—O—CH₃, —C(═O)—O—C₂H₅, —C(═O)—O—C(CH₃)₃, —O—C(═O)—CH₃, —O—C(═O)—C₂H₅, —O—C(═O)—C(CH₃)₃, —N(CH₃)₂, —N(C₂H₅)₂, —NH—CH₃, —NH—C₂H₅, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—C(CH₃)₃; and
R^{16a} is a radical selected from the group consisting of piperazinyl, thiomorpholinyl, azepanyl, morpholinyl, 2,3-dihydro-1H-isoindolyl, 2,3-dihydroindolyl, piperidinyl and pyrrolidinyl; where the radical may be substituted by 1, 2 or 3 substituents selected independently from the group consisting of —OH, methyl, ethyl, n-propyl, isopropyl, n-butyl, sec-butyl, isobutyl, tert-butyl, —C(═O)—O—CH₃, —C(═O)—O—C₂H₅, —C(═O)—O—C(CH₃)₃, —C(═O)—C₁₋₅-alkyl, —C(═O)—NH₂, —C(═O)—CH₃, —C(═O)—C₂H₅, —C(═O)—NH—CH₃, —C(═O)—NH—C₂H₅, —C(═O)—N—(CH₃)₂, —C(═O)—N—(C₂H₅)₂, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2, 3, 4 or 5 substituents selected independently from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CF₃, —S—CF₃, phenyl and —O-benzyl.

61. A compound as claimed in claim 49, wherein
R^{1a} is one of the following radicals 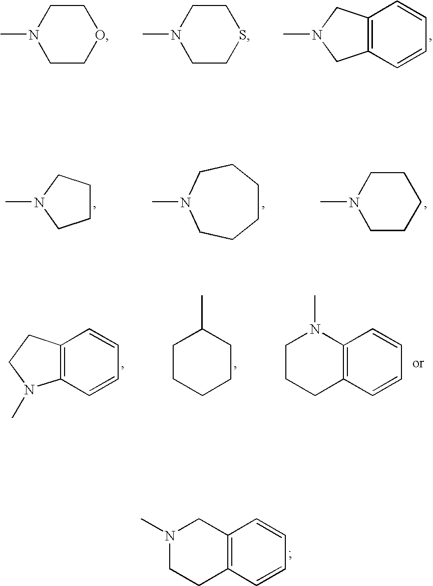
where the radical may be substituted by 1, 2 or 3 substituents selected independently from the group consisting of —OH, —O—CH₃, —O—C₂H₅, methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)— phenyl, —N(C₂H₁₅)-phenyl, —N[CH(CH₃)₂]-phenyl, —N(CH₃)-pyridinyl, —N(C₂H₅)— pyridinyl, —N[CH(CH₃)₂]-pyridinyl and phenyl; where the cyclic moiety of the phenyl radical may in each case be substituted by 1, 2 or 3 substituents selected independently from the group consisting of F, Cl, Br and —CF₃;
is one of the following radicals 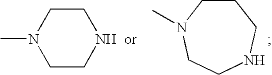
where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is an NR^{3a}R^{4a} group
or is an NR^{7a}—C(═O)—NR^{8a}R^{9a} group;
R^{2a} is CH—CH═C(CH₃)-Z^{a}, CH—CH═CH-Z^{a}, CH—CH═CH—S-Z^{a}, CH—CH═CH—O-Z^{a}, CH—CH═CH—N(CH₃)-Z^{a}, CH—C(CH₃)═CH-Z^{a}, CH—C(phenyl)=CH-Z^{a}, CH—CBr═CH-Z^{a}, CH—CCl═CH-Z^{a}, CH—CF═CH-Z^{a}, CH—C(OH)═CH-Z^{a}, CH—CH₂—CH₂-Z^{a}, CH—CH₂—CH(CH₃)-Z^{a} or CH—CH(CH₃)—CH₂-Z^{a};
R^{3a} is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
is a radical selected from the group consisting of cyclopentyl, cyclohexyl, cycloheptyl, cyclopentenyl, cyclohexenyl and cycloheptenyl; where the radical may in each case optionally be substituted by 1, 2, 3, 4 or 5 substituents selected independently from the group consisting of methyl, ethyl, n-propyl, isopropyl, n-butyl, sec-butyl, isobutyl, tert-butyl, —N(CH₃)₂, —N(C₂H₅)₂, —NH—CH₃, —NH—C₂H₅, —NH—CH(CH₃)₂, —NH—C(CH₃)₃, —NH-phenyl, —NH-pyridinyl, —N(CH₃)-phenyl, —N(C₂H₅)-phenyl, —N[CH(CH₃)₂]-phenyl, —N(CH₃)-pyridinyl, —N(C₂H₅)-pyridinyl, —N[CH(CH₃)₂]-pyridinyl;
or is a radical selected from the group consisting of phenyl, naphthyl, (1,3)-benzodioxolyl and (1,4)-benzodioxanyl; where the radical may be bonded via a —(CH₂)—, —(CH₂)—(CH₂) or —(CH₂)—(CH₂)—(CH₂) group and/or may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R^{4a} is a hydrogen radical
or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{7a} and R^{8a} are each independently
a hydrogen radical
or a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{9a} is a radical selected from the group consisting of phenyl, naphthyl, thiophenyl, furanyl, pyridinyl, thiazolyl and oxazolyl, where the radical may in each case optionally be substituted by 1, 2, 3, 4 or 5 substituents selected independently from the group consisting of F, Cl, Br, I, —CN, —CF₃, —SF₅, —O—CH₃, —O—C₂H₅, —O—CF₃, —S—CF₃, methyl, ethyl, n-propyl, isopropyl, n-butyl, sec-butyl, isobutyl and tert-butyl;
Z^{a} is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and n-eicosanyl;
is a radical selected from the group consisting of phenyl, naphthyl, phenanthrenyl, (1,3)-benzodioxolyl and (1,4)-benzodioxanyl, which is optionally substituted by 1, 2, 3, 4 or 5 identical or different substituents R^{12a};
or is a radical selected from the group consisting of thiophenyl, furanyl, pyrrolyl, pyrazolyl, pyrazinyl, pyridinyl, pyridazinyl, pyrimidinyl, quinolinyl and isoquinolinyl, which may optionally be substituted by 1, 2, 3, 4 or 5 identical or different substituents R^{12a};
R^{12a} is a radical selected from the group consisting of F, Cl, Br, —SF₅, —CF₃, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, methyl, ethyl, n-propyl, isopropyl, n-butyl, sec-butyl, tert-butyl, n-pentyl, n-hexyl, n-heptyl and n-octyl.

62. A compound as claimed in claim 49, wherein
R^{1a} is one of the following radicals 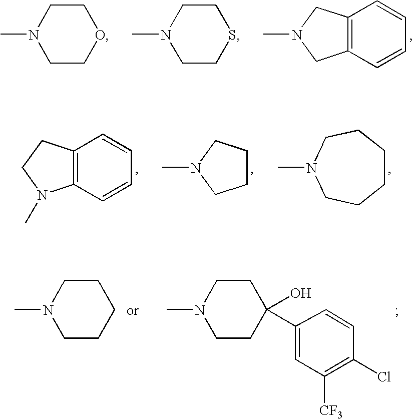
is the following radical 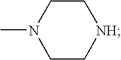
where the hydrogen atom of the —NH group may be replaced by a substituent selected from the group consisting of methyl, ethyl, n-propyl, isopropyl, n-butyl, sec-butyl, isobutyl, tert-butyl, pyridinyl, pyridazinyl, [1,2,5]-thiadiazolyl, benzyl and phenyl; where the cyclic moiety of the pyridinyl, pyridazinyl, [1,2,5]-thiadiazolyl, benzyl and phenyl radicals may in each case be substituted by 1, 2 or 3 substituents selected independently from the group consisting of F, Cl, Br, —CF₃, methyl, ethyl, n-propyl, isopropyl, n-butyl, sec-butyl, isobutyl, tert-butyl, —O—CH₃, —O—C₂H₅, —O—CF₃ and —S—CF₃;
is an NR^{3a}R^{4a} group
or is an NR^{7a}—C(═O)—NR^{8a}R^{9a} group;
R^{2a} is CH—CH═C(CH₃)-Z^{a}, CH—CH═CH-Z^{a}, CH—CH═CH—S-Z^{a}, CH—C(CH₃)═CH-Z^{a}, CH—C(phenyl)=CH-Z^{a}, CH—CBr═CH-Z^{a}, CH—CCl═CH-Z^{a}, CH—CF═CH-Z^{a}, CH—CH₂—CH₂-Z^{a}, CH—CH₂—CH(CH₃)-Z^{a} or CH—CH(CH₃)—CH₂-Z^{a};
R^{3a} is a radical selected from the group consisting of methyl, —CH₂—O—CH₃, —CH₂—S—CH₃, ethyl, —CH₂—CH₂—S—CH₃, —CH₂—CH₂—O—CH₃, —CH₂—CH₂—N(CH₃)—CH₃, n-propyl, —CH₂—CH₂—CH₂—S—CH₃, —CH₂—CH₂—CH₂—O—CH₃, —CH₂—CH₂—CH₂—N(CH₃)—CH₃, isopropyl, n-butyl, sec-butyl, isobutyl, tert-butyl, 1,1-dimethylpropyl, n-pentyl, sec-pentyl, n-hexyl and n-heptyl;
or is a radical selected from the group consisting of phenyl, naphthyl, benzyl, (1,3)-benzodioxolyl and (1,4)-benzodioxanyl; where the radical may in each case optionally be substituted by 1, 2, 3, 4 or 5 substituents selected independently from the group consisting of F, Cl, Br, —CF₃, —O—CF₃, —OCH₃, —OC₂H₅, methyl, ethyl, n-propyl, isopropyl, n-butyl and tert-butyl;
R^{4a} is a hydrogen radical or is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl and n-heptyl;
R^{7a} and R^{8a} are each a hydrogen radical;
R^{9a} is a phenyl radical which may be substituted by 1, 2, 3, 4 or 5 substituents selected independently from the group consisting of F, Cl, Br and —CF₃;
Z^{a} is a radical selected from the group consisting of methyl, ethyl, n-propyl, isopropyl, n-butyl, sec-butyl, isobutyl, tert-butyl, 1,1-dimethylpropyl, n-pentyl, sec-pentyl, n-hexyl, n-heptyl and n-octyl;
or is a phenyl or naphthyl radical which may be substituted by 1, 2, 3, 4 or 5 identical or different substituents R^{12a};
R^{12a} is a radical selected from the group consisting of F, Cl, Br, I, —CN, —CF₃, —SF₅, —OH, —O—CH₃, —O—C₂H₅, —O—CH₂—CH₂—CH₃, —O—CH₂—CH₂—CH₂—CH₃, —O—C(CH₃)₃, —O—CH₂—CH₂—CH₂—CH₂—CH₃, —O—CH₂—CH₂—C(CH₃)₃, —O—CH═CH₂, —O—CH₂—CH═CH₂, —NH₂, —O—CF₃, —S—CF₃, —SH, —S—CH₃, —S—C₂H₅, —S—CH₂—CH₂—CH₃, —S—CH₂—CH₂—CH₂—CH₃, —S—C(CH₃)₃, methyl, ethyl, n-propyl, isopropyl, n-butyl, sec-butyl, tert-butyl, n-pentyl, n-hexyl, n-heptyl and n-octyl.

63. A compound as claimed in claim 49, selected from the group consisting of:
1. 5-[3-(4-methoxyphenyl)but-2-enylidene]-2-morpholin-4-ylthiazol-4-one;
2. 5-[3-(4-fluorophenyl)allylidene]-2-morpholin-4-ylthiazol-4-one;
3. 2-morpholin-4-yl-5-(3-m-tolylallylidene)thiazol-4-one;
4. 2-morpholin-4-yl-5-(3-p-tolylallylidene)thiazol-4-one;
5. 2-morpholin-4-yl-5-octylidenethiazol-4-one;
6. 5-butylidene-2-morpholin-4-ylthiazol-4-one;
7. 5-hexylidene-2-morpholin-4-ylthiazol-4-one;
8. 2-morpholin-4-yl-5-(3-p-tolylbut-2-enylidene)thiazol-4-one;
9. 5-[3-(4-tert-butylphenyl)but-2-enylidene]-2-morpholin-4-ylthiazol-4-one;
10. 5-[3-(4-tert-butylphenyl)allylidene]-2-[4-(3-methoxyphenyl)piperazin-1-yl]thiazol-4-one;
11. 5-[3-(4-hydroxy-3-methoxyphenyl)allylidene]-2-thiomorpholin-4-ylthiazol-4-one;
12. 2-morpholin-4-yl-5-[3-(4-trifluoromethylphenyl)but-2-enylidene]thiazol-4-one;
13. 2-morpholin-4-yl-5-[3-(3-trifluoromethylphenyl)but-2-enylidene]thiazol-4-one;
14. 5-decylidene-2-morpholin-4-ylthiazol-4-one;
15. 5-[3-(4-tert-butylphenyl)allylidene]-2-morpholin-4-ylthiazol-4-one;
16. 5-[3-(4-tert-butylphenyl)allylidene]-2-[4-(3-chloropyridin-2-yl)piperazin-1-yl]thiazol-4-one;
17. 5-[3-(4-tert-butylphenyl)allylidene]-2-[4-(2-fluorophenyl)piperazin-1-yl]thiazol-4-one;
18. 5-[3-(4-tert-butylphenyl)allylidene]-2-[4-(4-chloro-[1,2,5]thiadiazol-3-yl)-piperazin-1-yl]thiazol-4-one;
19. 5-[3-(4-tert-butylphenyl)allylidene]-2-[4-(6-chloropyridin-2-yl)piperazin-1-yl]thiazol-4-one;
20. 5-[3-(4-tert-butylphenyl)allylidene]-2-[4-(6-methylpyridazin-3-yl)piperazin-1-yl]thiazol-4-one;
21. 2-[(benzo[1,3]dioxol-5-ylmethyl)amino]-5-[3-(4-tert-butylphenyl)-allylidene]thiazol-4-one;
or a pharmaceutically acceptable salt or solvate thereof.

64. A process for preparing 2,5-disubstituted thiazol-4-one corresponding to formula Ia as claimed in claim 49, wherein
2-aminothiazol-4-one is reacted in acetic acid in the presence of at least one salt selected from the group consisting of sodium acetate and potassium acetate or in a reaction medium selected from the group consisting of methanol, ethanol, isopropanol and n-butanol in the presence of at least one organic base selected from the group consisting of triethylamine, pyridine, diisopropylamine and N-methylmorpholine with at least one compound of the general formula R—C(═O)—H, in which R is —CH═C(CH₃)_W^{a}-Z^{a}, —CH═CH—W^{a}-Z^{a}, —C(CH₃)═CH_W^{a}-Z^{a}, —C(phenyl)=CH—W^{a}-Z^{a}, —CBr═CH—W^{a}-Z^{a}, —CCl═CH—W^{a}-Z^{a}, —CF═CH—W^{a}-Z^{a}, —C(OH)═CH_W^{a}-Z^{a}, —CH₂—CH₂—W^{a}-Z^{a}, —CH₂—CH(CH₃)—W^{a}-Z^{a} or —CH(CH₃)—CH₂—W^{a}-Z^{a}, where W^{a} and Z^{a} are each as defined in claim 49, to give at least one compound of the general formula Ia, 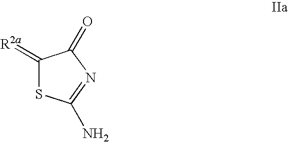
where R^{2a} is as defined in one or more of claims 17 to 29, and the latter is optionally purified and/or isolated, and at least one compound of the general formula Ia is reacted in a reaction medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{3a} or of the general formula LG-R^{7a} or of the general formula LG-R^{5a}, in which LG is in each case a leaving group, preferably a halogen atom, more preferably a chlorine atom, and R^{3a}, R^{5a} and R^{7a} are each as defined in claim 49 to give at least one compound of the general formula Ia in which R^{2a} is as defined above and R^{1a} is an NHR^{3a}—, NHR^{5a}— or NHR^{7a} group, and the latter is optionally purified and/or isolated, and optionally at least one compound of the general formula Ia in which R^{2a} is as defined above and R^{1a} is an NHR^{7a} group is reacted in a reaction medium with at least one isocyanate of the general formula R^{8a}—N═C═O, in which R^{8a} is as defined in one or more of claims 17 to 29, optionally in the presence of at least one base, preferably in the presence of at least one base selected from the group consisting of triethylamine, 4,4-dimethylaminopyridine and diisopropylethylamine, to give at least one compound of the general formula Ia in which R^{2a} is as defined above and R^{1a} is an NR^{7a}—C(═O)—NHR^{8a} group, and the latter is optionally purified and/or isolated;
and optionally at least one compound of the general formula Ia in which R^{2a} is as defined above and R^{1a} is an NR^{7a}—C(═O)—NHR^{8a} group is reacted in a reaction medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{9a} in which LG is a leaving group, preferably a halogen atom, more preferably a chlorine atom, and R^{9a} is as defined in claim 49 to give at least one compound of the general formula Ia in which R^{2a} is as defined above and R^{1a} is an NR^{7a}—C(═O)—NR^{8a}R^{9a} group, and the latter is optionally purified and/or isolated; or
optionally at least one compound of the general formula IIa in which R^{2a} is as defined above and R^{1a} is an NHR^{5a} group is reacted in a reaction medium, optionally in the presence of at least one base, with at least one compound of the general formula R^{6a}—C(═O)-LG in which R^{6a} is as defined in claim 49 and LG is a leaving group, preferably a halogen radical, or in a reaction medium in the presence of at least one coupling reagent, optionally in the presence of at least one base, with a compound of the general formula R^{6a}—C(═O)—OH in which R^{6a} is as defined in claim 49, to give at least one compound of the general formula Ia in which R^{2a} is as defined above and R^{1a} is an NR^{5a}—C(═O)—R^{6a} group, and the latter is optionally purified and/or isolated or
optionally at least one compound of the general formula Ia in which R^{2a} is as defined above and R^{1a} is an NHR^{3a} group is reacted in a reaction medium in the presence of at least one base, preferably in the presence of at least one metal hydride salt or of a metal alkoxide salt, more preferably in the presence of a metal hydride salt or of a metal alkoxide salt selected from the group consisting of sodium hydride, potassium hydride, potassium tert-butoxide, sodium tert-butoxide, potassium methoxide, sodium methoxide, sodium ethoxide and potassium ethoxide, with at least one compound of the general formula LG-R^{4a} in which LG is a leaving group, preferably a halogen atom, more preferably a chlorine atom, and R^{4a} is as defined in claim 49 to give at least one compound of the general formula Ia in which R^{2a} is as defined above and R^{1a} is an NR^{3a}R^{4a} group, and the latter is optionally purified and/or isolated.

65. A process for preparing a 2,5-disubstituted thiazol-4-one compound corresponding to formula Ia as claimed in claim 49, wherein
a compound of the formula R^{1a}—CN in which R^{1a} is as defined in claim 49 with the exception of an NR^{3a}R^{4a} group, of an NR^{5a}—C(═O)—R^{6a} group and of an NR^{7a}—C(═O)—NR^{8a}R⁹ group, is reacted in a reaction medium selected from the group consisting of methanol, ethanol, isopropanol and n-butanol, in the presence of at least one organic base selected from the group consisting of triethylamine, pyridine, diisopropylamine and N-methylmorpholine or in pyridine as a reaction medium, with thioglycolic acid to give at least one compound of the general formula IIa, 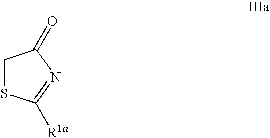
in which R^{1a} is as defined in one or more of claims 17 to 29 with the exception of an NR^{3a}R^{4a} group, of an NR^{5a}—C(═O)—R^{6a} group and of an NR^{7a}—C(═O)_NR^{8a}R^{9a} group, and the latter is optionally purified and/or isolated;
or at least one compound of the general formula H₂N—C(═S)—NHR^{1a} in which R^{1a} is as defined in claim 49 with the exception of an NR^{3a}R^{4a} group, of an NR^{5a}—C(═O)—R^{6a} group and of an NR^{7a}—C(═O)—NR^{8a}R^{9a} group, is reacted in a reaction medium selected from the group consisting of methanol, ethanol, isopropanol and n-butanol, optionally in the presence of at least one organic base selected from the group consisting of triethylamine, pyridine, diisopropylamine and N-methylmorpholine, with chloroacetic acid to give at least one compound of the general formula IIIa in which R^{1a} is as defined in claim 49 with the exception of an NR^{3a}R^{4a} group, of an NR^{5a}—C(═O)—R^{6a} group and of an NR^{7a}—C(═O)_NR^{8a}R^{9a} group, and the latter is optionally purified and/or isolated;
and at least one compound of the general formula IIIa is reacted in acetic acid in the presence of at least one salt selected from the group consisting of sodium acetate and potassium acetate or in a reaction medium selected from the group consisting of methanol, ethanol, isopropanol and n-butanol in the presence of at least one organic base selected from the group consisting of triethylamine, pyridine, diisopropylamine and N-methylmorpholine with at least one compound of the general formula R—C(═O)—H in which R is —CH═C(CH₃)_W^{a}-Z^{a}, —CH═CH—W^{a}-Z^{a}, —C(CH₃)═CH_W^{a}-Z^{a}, —C(phenyl)=CH_W^{a}-Z^{a}, —CBr═CH—W^{a}-Z^{a}, —CCl═CH_W^{a}-Z^{a}, —CF═CH_W^{a}-Z^{a}, —C(OH)═CH_W^{a}-Z^{a}, —CH₂—CH₂—W^{a}-Z^{a}, —CH₂—CH(CH₃)—W^{a}-Z^{a} or —CH(CH₃)—CH₂—W^{a}-Z^{a}, where W^{a} and Z^{a} are each as defined in claim 49, to give at least one compound of the general formula Ia in which R^{1a} is as defined in claim 49 with the exception of an NR^{3a}R^{4a} group, of an NR^{5a}—C(═O)—R^{6a} group and of an NR^{7a}—C(═O)—NR^{8a}R^{9a} group and R^{2a} is as defined in claim 49.

66. A pharmaceutical composition comprising a compound as claimed in claim 49, and at least one physiologically compatible excipient.

67. A method of treating or inhibiting a disorder or disease state selected from the group consisting of pain; migraine; depression; neuropathy; neural injuries; neurodegenerative disorders; cognitive dysfunctions; epilepsy; respiratory disorders; coughing; urinary incontinence; overactive bladder; stomach ulcers; irritable bowel syndrome; stroke; eye irritation; skin irritation; neurotic skin disorders; inflammations; diarrhea; pruritus; eating disorders; medicament, drug or alcohol dependency; medicament, drug or alcohol abuse; withdrawal symptoms due to medicament, drug or alcohol dependence or addiction; development of tolerance to medicaments; or for diuresis; antinatriuresis; influencing the cardiovascular system; enhancing vigilance; increasing libido; modulating movement activity; anxiolysis; local anesthesia or inhibiting undesired side effects triggered by the administration of Vanilloid receptor 1 (VR1/TRPV1 receptor) agonists; in a subject in need thereof, said method comprising administering to said subject a pharmacologically effective amount of a compound as claimed in claim 49.

68. A method as claimed in claim 33, wherein said disorder or disease state is pain selected from the group consisting of acute pain, chronic pain, visceral pain, neuropathic pain and joint pain; a neurodegenerative disorder selected from the group consisting of multiple sclerosis, Alzheimer's disease, Parkinson's disease and Huntington's disease; a cognitive deficiency; memory impairment; a respiratory disorder selected from the group consisting of asthma and lung inflammation; inflammation of the bowel; an eating disorder selected from the group consisting of bulimia, cachexia, anorexia and obesity; development of tolerance to natural or synthetic opioids; or for inhibiting side effects selected from the group consisting of hyperthermia, hypertension and narrowing of the bronchia, triggered by the administration of Vanilloid receptor 1 agonists selected from the group consisting of capsaicin, resiniferatoxin, olvanil, arvanil, SDZ-249665, SDZ-249482, nuvanil and capsavanil (DA-5018).
